(19) United States
(12) Patent Application Publication
(10) Pub. No.: US 2013/0210804 A1
(43) Pub. Date: Aug. 15, 2013

(54) TRICYCLIC MGLUR5 RECEPTOR MODULATORS

(71) Applicants: Neville J. Anthony, Northborough, MA (US); Robert P. Gomez, Perkasie, PA (US); Jing Li, Lansdale, PA (US); Swati P. Mercer, Philadelphia, PA (US); Anthony J. Roecker, North Wales, PA (US); Craig A. Stump, Pottstown, PA (US); Theresa M. Williams, Harleysville, PA (US)

(72) Inventors: Neville J. Anthony, Northborough, MA (US); Robert P. Gomez, Perkasie, PA (US); Jing Li, Lansdale, PA (US); Swati P. Mercer, Philadelphia, PA (US); Anthony J. Roecker, North Wales, PA (US); Craig A. Stump, Pottstown, PA (US); Theresa M. Williams, Harleysville, PA (US)

(21) Appl. No.: 13/880,171

(22) PCT Filed: Oct. 20, 2011
(86) PCT No.: PCT/US11/56981

§ 371 Date: Apr. 18, 2013

Related U.S. Application Data
(60) Provisional application No. 61/406,329, filed on Oct. 25, 2010.

(51) Int. Cl. C07D 513/04; C07D 498/04; C07D 471/14; C07D 471/04

(52) U.S. Cl. CPC C07D 513/04; C07D 471/04; C07D 498/04; C07D 471/14
USPC 514/211.04; 540/522; 514/212.05; 540/496; 514/220; 540/488; 540/521

(57) ABSTRACT

The present invention is directed to tricyclic compounds which are positive allosteric modulators of metabotropic glutamate receptors, particularly the mGluR5 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### BACKGROUND OF THE INVENTION

The excitatory amino acid L-glutamic acid (sometimes referred to simply as L-glutamate or glutamate) through its many receptors mediates most of the excitatory neurotransmission within the mammalian central nervous system (CNS). The excitatory amino acids, including glutamate, are of great physiological importance, playing a role in a variety of physiological processes, such as long-term potentiation (learning and memory), the development of synaptic plasticity, motor control, respiration, cardiovascular regulation, and sensory perception. Glutamate acts via at least two distinct classes of receptors. One class is composed of the ionotropic glutamate (iGlu) receptors that act as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life.

The present invention relates to modulators of metabotropic glutamate receptors, in particular subtype 5 (“mGluR5”) receptors. The mGluR receptors belong to the Type III G-protein coupled receptor (GPCR) superfamily. This superfamily of GPCRs include the calcium-sensing receptors, GABA B receptors and pheromone receptors, which are unique in that they are activated by binding of effectors to the amino-terminus portion of the receptor protein. The mGlu receptors are thought to mediate glutamate's demonstrated ability to modulate intracellular signal transduction pathways. They have been demonstrated to be localized both pre- and post-synaptically where they can regulate neurotransmitter release, either glutamate or other neurotransmitters, or modify the post-synaptic response of neurotransmitters, respectively.

At present, there are eight distinct mGlu receptors that have been positively identified, cloned, and their sequences reported. These are further subdivided into three groups (Groups I, II and III) based on their amino acid sequence homology, their ability to effect certain signal transduction mechanisms, and their known pharmacological properties. Activation of mGluRs lead to a large variety of intracellular responses and activation of different transductional cascades. Among mGluR members, the mGluR5 subtype is of high interest for counterbalancing the deficit or excesses of neurotransmission in neuropsychatric diseases. mGluR5 belongs to Group I and its activation initiates cellular responses through G-protein mediated mechanisms. mGluR5 is coupled to phospholipase C and stimulates phosphoinositide hydrolysis and intracellular calcium mobilization. In the CNS, mGluR5 receptors are abundant mainly throughout cortex, hippocampus, caudate-putamen and nucleus accumbens. As these brain areas have been shown to be involved in emotion, motivational processes and in numerous aspects of cognitive function, mGluR5 modulators are predicted to be of therapeutic interest.

It has become increasingly clear that there is a link between modulation of excitatory amino acid receptors, including the glutamatergic system, through changes in glutamate release or alteration in postsynaptic receptor activation, and a variety of neurological and psychiatric disorders. For example, a variety of potential clinical indications have been suggested to be targets for the development of subtype selective mGluR modulators. These include epilepsy, neuropathic and inflammatory pain, numerous psychiatric disorders (e.g. anxiety and schizophrenia), movement disorders (e.g. Parkinson disease), neuroprotection (stroke and head injury), migraine and addiction/drug dependency. The medical consequences of such glutamate dysfunction make the abatement of these neurological processes an important therapeutic goal.

### SUMMARY OF THE INVENTION

The present invention is directed to tricyclic compounds which are positive allosteric modulators of metabotropic glutamate receptors, particularly the mGluR5 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### DETAILED DESCRIPTION OF THE INVENTION

The present invention is directed to compounds of the formula I:

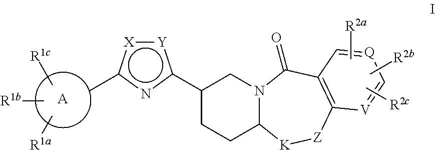

wherein:
A is selected from the group consisting of phenyl, naphthyl and heteroaryl;
X is selected from N, O, S and C(R⁸),
Y is selected from N, O, S and C(R⁸),
wherein X is N and Y is O, to form a oxadiazole ring, or
X is O and Y is N, to form a oxadiazole ring, or
X is C(R⁸) and Y is S to form a thiazole ring, or
X is S and Y is C(R⁸) to form a thiazole ring, or
X is C(R⁸) and Y is N to form an imidazole ring, or
X is N and Y is C(R⁸) to form an imidazole ring, or
X is C(R⁸) and Y is O to form an oxazole ring, or
X is O and Y is C(R⁸) to form an oxazole ring, or
X is N and Y is N to form a triazole ring;
   Q is selected from C and N,
   V is selected from C and N,

### K is —CH₂—,

Z is selected from —CH₂—, —CH(CH₃)—, —CHF—, —CF₂—, —CH(OH)—, —O—, —S—, —S(O)—, —NH—, and —N(CH₃)—, or
K is —CH(OH)— and Z is —CH(OH)—, or
K and Z taken together form —CH═CH—, or
K and Z taken together form a cyclopropyl ring;
   R^{1a}, R^{1b} and R^{1c} may be absent if the valency of A does not permit such substitution and are independently selected from the group consisting of:
(1) hydrogen,
(2) halogen,
(3) hydroxyl,
(4) —(C═O)ₘ—Oₙ—C₁₋₆alkyl, where m is 0 or 1, n is 0 or 1 (wherein if m is 0 or n is 0, a bond is present) and where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘ—C₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heteroaryl, where the heteraryl is unsubstituted or substituted with one or more substituents selected from R¹³,
(10) —(C═O)ₘ—NR¹⁰R¹¹, wherein R¹⁰ and R¹¹ are independently selected from the group consisting of:
   (a) hydrogen,
   (b) C₁₋₆alkyl, which is unsubstituted or substituted with R¹⁴,
   (c) C₃₋₆alkenyl, which is unsubstituted or substituted with R¹⁴,
   (d) C₃₋₆alkynyl, which is unsubstituted or substituted with R¹⁴,
   (e) C₃₋₆cycloalkyl which is unsubstituted or substituted with R¹⁴,
   (f) phenyl, which is unsubstituted or substituted with R¹⁴, and
   (g) heteroaryl, which is unsubstituted or substituted with R¹⁴,
(11) —S(O)₂—NR¹⁰R¹¹,
(12) —S(O)_{q}—R¹², where q is 0, 1 or 2 and where R¹² is selected from the definitions of R¹⁰ and R¹¹,
(13) —CO₂H,
(14) —CN, and
(15) —NO₂;
   R^{2a}, R^{2b} and R^{2c} are independently selected from the group consisting of:
(1) hydrogen,
(2) halogen,
(3) hydroxyl,
(4) —(C═O)ₘ—Oₙ—C₁₋₆alkyl, where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘ—C₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heteroaryl, where the heteroaryl is unsubstituted or substituted with one or more substituents selected from R¹³,
(10) —(C═O)ₘ—NR¹⁰R¹¹,
(11) —S(O)₂—NR¹⁰R¹¹,
(12) —S(O)_{q}—R¹²,
(13) —CO₂H,
(14) —CN, and
(15) —NO₂;
   R⁸ is selected from the group consisting of:
(1) hydrogen,
(2) halogen,
(3) C₁₋₆alkyl, which is unsubstituted or substituted with R¹³,
(4) C₃₋₆alkenyl, which is unsubstituted or substituted with R¹³,
(5) C₃₋₆alkynyl, which is unsubstituted or substituted with R¹³,
(6) C₃₋₆cycloalkyl which is unsubstituted or substituted with R¹³,
(7) phenyl, which is unsubstituted or substituted with R¹³, and
(6) heteroaryl, which is unsubstituted or substituted with R¹³,
   R¹³ is selected from the group consisting of
(1) halogen,
(2) hydroxyl,
(3) —(C═O)ₘ—Oₙ—C₁₋₆alkyl, where the alkyl is unsubstituted or substituted with one or more substituents selected from R¹⁴,
(4) —Oₙ—(C₁₋₃)perfluoroalkyl,
(5) —(C═O)ₘ—Oₙ—C₃₋₆cycloalkyl, where the cycloalkyl is unsubstituted or substituted with one or more substituents selected from R¹⁴,
(6) —(C═O)ₘ—C₂₋₄alkenyl, where the alkenyl is unsubstituted or substituted with one or more substituents selected from R¹⁴,
(7) —(C═O)ₘ—C₂₋₄alkynyl, where the alkynyl is unsubstituted or substituted with one or more substituents selected from R¹⁴,
(8) —(C═O)ₘ—Oₙ-phenyl or —(C═O)ₘ—Oₙ-napthyl, where the phenyl or naphthyl is unsubstituted or substituted with one or more substituents selected from R¹⁴,
(9) —(C═O)ₘ—Oₙ-heteroaryl, where the heteroaryl is unsubstituted or substituted with one or more substituents selected from R¹⁴,
(10) —(C═O)ₘ—NR¹⁰R¹¹,
(11) —S(O)₂—NR¹⁰R¹¹,
(12) —S(O)_{q}—R¹²,
(13) —CO₂H,
(14) —CN, and
(15) —NO₂;
   R¹⁴ is selected from the group consisting of:
(1) hydroxyl,
(2) halogen,
(3) C₁₋₆alkyl,
(4) —C₃₋₆cycloalkyl,
(5) —O—C₁₋₆alkyl,
(6) —O(C═O)—C₁₋₆alkyl,
(7) —NH—C₁₋₆alkyl,
(8) phenyl,
(9) heteroaryl,
(10) —CO₂H, and
(11) —CN;
   or a pharmaceutically acceptable salt thereof.

An embodiment of the present invention includes compounds of the formula I′:

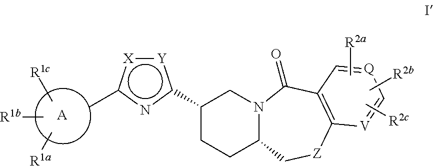

wherein A, X, Y, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds of the formula I″:

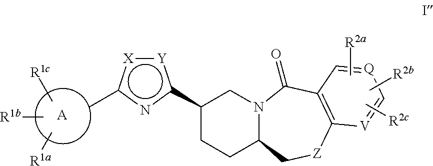

wherein A, X, Y, Q, V, Z, R^{1a} a, R^{1b} b, R^{1c}, R^{2a}, R^{2b} and R^{2c} are defined herein; or a pharmaceutically acceptable salt thereof.

An embodiment of the present invention includes compounds of the formula Ia:

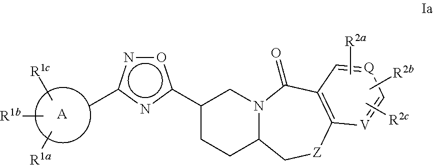

wherein A, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds of the formula Ia′:

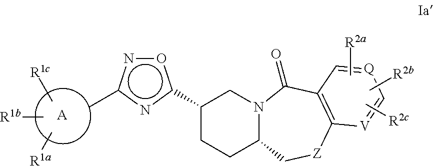

wherein A, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds of the formula Ia″:

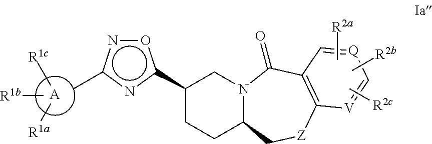

wherein A, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds of the formula Ib:

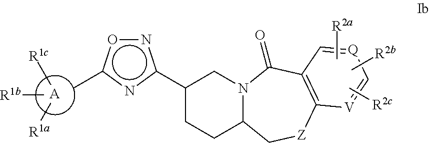

wherein A, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds of the formula Ib′:

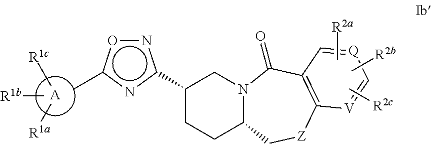

wherein A, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds of the formula Ib″:

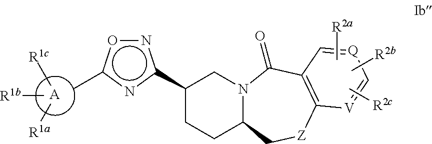

wherein A, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds of the formula Ic:

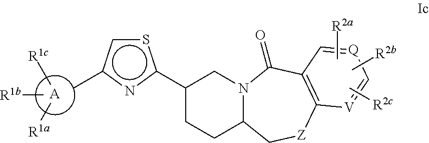

wherein A, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds of the formula Ic′:

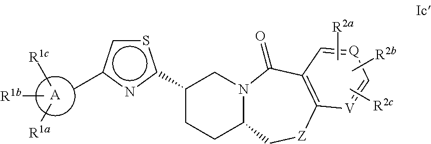

wherein A, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds of the formula Ic″:

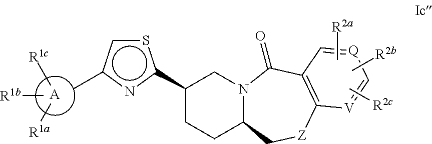

wherein A, Q, V, Z, R^{1a}, R^{1b}, R^{1c}, R^{2a}, R^{2b} and R^{2c} are defined herein; or a pharmaceutically acceptable salt thereof.

An embodiment of the present invention includes compounds wherein A is selected from the group consisting of phenyl, pyridyl and pyrrolyl. An embodiment of the present invention includes compounds wherein A is phenyl. An embodiment of the present invention includes compounds wherein A is heteroaryl. An embodiment of the present invention includes compounds wherein A is pyridyl. An embodiment of the present invention includes compounds wherein A is pyrrolyl. An embodiment of the present invention includes compounds wherein A is pyrimidinyl. An embodiment of the present invention includes compounds wherein A is thiophenyl.

An embodiment of the present invention includes compounds wherein X is N and Y is O, to form a oxadiazole ring. An embodiment of the present invention includes compounds wherein X is O and Y is N, to form a oxadiazole ring. An embodiment of the present invention includes compounds wherein X is C(R⁸) and Y is O to form an oxazole ring. An embodiment of the present invention includes compounds wherein X is C(R⁸) and Y is S to form a thiazole ring.

An embodiment of the present invention includes compounds wherein Q is C and V is C. An embodiment of the present invention includes compounds wherein Q is N and V is C. An embodiment of the present invention includes compounds wherein Q is C and V is N.

An embodiment of the present invention includes compounds wherein Z is —CH₂—. An embodiment of the present invention includes compounds wherein Z is —CF₂—. An embodiment of the present invention includes compounds wherein Z is —O—. An embodiment of the present invention includes compounds wherein Z is —S—. An embodiment of the present invention includes compounds wherein Z is —S(O)—. An embodiment of the present invention includes compounds wherein Z is —NH—. An embodiment of the present invention includes compounds wherein K is —CH₂—,

An embodiment of the present invention includes compounds wherein R^{1a}, R^{1b} and R^{1c} are independently selected from the group consisting of:
(1) hydrogen,
(2) halogen,
(3) hydroxyl,
(4) C₁₋₆alkyl, which is unsubstituted or substituted with halogen, hydroxyl, phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆alkyl or —NO₂,
(7) phenyl, which is unsubstituted or substituted with halogen, hydroxyl, C₁₋₆alkyl, —O—C₁₋₆alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, or —O—C₁₋₆alkyl.

An embodiment of the present invention includes compounds wherein R^{1a}, R^{1b} and R^{1c} are independently selected from the group consisting of:
(1) hydrogen,
(2) halogen,
(3) hydroxyl,
(4) C₁₋₆alkyl, which is unsubstituted or substituted with halogen, hydroxyl or phenyl or napthyl, and
(5) —O—C₁₋₆alkyl, which is unsubstituted or substituted with halogen, hydroxyl or phenyl.

An embodiment of the present invention includes compounds wherein R^{1a}, R^{1b} and R^{1c} are independently selected from the group consisting of:
(1) hydrogen,
(2) halogen, and
(3) C₁₋₆alkyl.

An embodiment of the present invention includes compounds wherein R^{1a}, R^{1b} and R^{1c} are independently selected from the group consisting of:
(1) hydrogen,
(2) chloro,
(3) fluororo, and
(4) methyl.

An embodiment of the present invention includes compounds wherein A is phenyl, pyridyl or pyrrolyl and R^{1a}, R^{1b} and R^{1c} are independently selected from the group consisting of:
(1) hydrogen,
(2) chloro,
(3) fluororo, and
(4) methyl.

An embodiment of the present invention includes compounds wherein A is phenyl and wherein R^{1a} is halogen, R^{1b} is hydrogen and R^{1c} is hydrogen. An embodiment of the present invention includes compounds wherein A is phenyl and wherein R^{1a} is fluoro, R^{1b} is hydrogen and R^{1c} is hydrogen. An embodiment of the present invention includes compounds wherein A is phenyl and wherein R^{1a} is chloro, R^{1b} is hydrogen and R^{1c} is hydrogen. An embodiment of the present invention includes compounds wherein A is phenyl and wherein R^{1a} is methyl, R^{1b} is hydrogen and R^{1c} is hydrogen.

An embodiment of the present invention includes compounds wherein A is pyridyl and wherein R^{1a} is halogen, R^{1b} is hydrogen and R^{1c} is hydrogen. An embodiment of the present invention includes compounds wherein A is pyridyl and wherein R^{1a} is fluoro, R^{1b} is hydrogen and R^{1c} is hydrogen. An embodiment of the present invention includes compounds wherein A is pyridyl and wherein R^{1a} is chloro, R^{1b} is hydrogen and R^{1c} is hydrogen. An embodiment of the present invention includes compounds wherein A is pyridyl and wherein R^{1a} is methyl, R^{1b} is hydrogen and R^{1c} is hydrogen.

An embodiment of the present invention includes compounds wherein A is pyrrolyl and wherein R^{1a} is halogen, R^{1b} is hydrogen and R^{1c} is hydrogen. An embodiment of the present invention includes compounds wherein A is pyrrolyl and wherein R^{1a} is fluoro, R^{1b} is hydrogen and R^{1c} is hydrogen. An embodiment of the present invention includes compounds wherein A is pyrrolyl and wherein R^{1a} is chloro, R^{1b} is hydrogen and R^{1c} is hydrogen. An embodiment of the present invention includes compounds wherein A is pyrrolyl and wherein R^{1a} is methyl, R^{1b} is hydrogen and R^{1c} is hydrogen.

An embodiment of the present invention includes compounds wherein R^{2a}, R^{2b} and R^{2c} are independently selected from the group consisting of
(1) hydrogen,
(2) halogen,
(3) hydroxyl,
(4) C₁₋₆alkyl, which is unsubstituted or substituted with halogen, hydroxyl or phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆alkyl or —NO₂,
(7) phenyl, which is unsubstituted or substituted with halogen, hydroxyl, C₁₋₆alkyl, or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, or —O—C₁₋₆alkyl.

An embodiment of the present invention includes compounds wherein R^{2a}, R^{2b} and R^{2c} are independently selected from the group consisting of:
(1) hydrogen,
(2) halogen,
(3) hydroxyl,
(4) C₁₋₆alkyl, which is unsubstituted or substituted with halogen, hydroxyl or phenyl,
(5) —O—C₁₋₆alkyl, which is unsubstituted or substituted with halogen, hydroxyl or phenyl, and
(6) —NH—C₁₋₆alkyl, or —N(C₁₋₆alkyl)(C₁₋₆alkyl), which is unsubstituted or substituted with halogen.

An embodiment of the present invention includes compounds wherein R^{2a}, R^{2b} and R^{2c} are independently selected from the group consisting of:
(1) hydrogen,
(2) halogen,
(3) C₁₋₆alkyl, which is unsubstituted or substituted with halogen,
(4) —O—C₁₋₆alkyl, which is unsubstituted or substituted with halogen, and
(5) —NH—C₁₋₆alkyl, or —N(C₁₋₆alkyl)(C₁₋₆alkyl), which is unsubstituted or substituted with halogen.

An embodiment of the present invention includes compounds wherein R^{2a}, R^{2b} and R^{2c} are independently selected from the group consisting of:
(1) hydrogen,
(2) chloro,
(3) fluoro,
(4) bromo,
(5) methoxy,
(6) t-butoxy,
(7) difluoromethyl, and
(8) trifluoromethyl.

An embodiment of the present invention includes compounds wherein R^{2a}, R^{2b} and R^{2c} are independently selected from the group consisting of:
(1) hydrogen,
(2) chloro,
(3) fluoro, and
(4) methoxy.

An embodiment of the present invention includes compounds wherein R^{2a} is halogen or methoxy, R^{2b} is hydrogen and R^{2c} is hydrogen. An embodiment of the present invention includes compounds wherein R^{2a} is fluoro, R^{2b} is hydrogen and R^{2c} is hydrogen. An embodiment of the present invention includes compounds wherein R^{2a} is chloro, R^{2b} is hydrogen and R^{2c} is hydrogen. An embodiment of the present invention includes compounds wherein R^{2a} is methoxy, R^{2b} is hydrogen and R^{2c} is hydrogen. An embodiment of the present invention includes compounds wherein R^{2a} is fluororo, R^{2b} is fluoro and R^{2c} is hydrogen. An embodiment of the present invention includes compounds wherein R^{2a} is fluoro, R^{2b} is methoxy and R^{2c} is hydrogen. An embodiment of the present invention includes compounds wherein R^{2a} is methoxy, R^{2b} is methoxy and R^{2c} is hydrogen.

An embodiment of the present invention includes compounds wherein is R⁸ hydrogen.

Specific embodiments of the present invention include a compound which is selected from the group consisting of the subject compounds of the Examples herein and pharmaceutically acceptable salts thereof and individual enantiomers and diastereomers thereof.

As appreciated by those of skill in the art, halogen or halo as used herein are intended to include fluorine, chlorine, bromine and iodine. Similarly, “alkyl”, as well as other groups having the prefix “alk”, such as alkoxy, alkanoyl, means carbon chains which may be linear or branched or combinations thereof. C₁₋₆, as in C₁₋₆alkyl is defined to identify the group as having 1, 2, 3, 4, 5 or 6 carbons in a linear or branched arrangement, such as methyl, ethyl, n-propyl, iso-propyl, n-butyl, iso-butyl, sec-butyl, tert-butyl, pentyl, hexyl, and the like. “Alkylene” means a straight or branched chain of carbon atoms with a group substituted at both ends, such as —CH₂CH₂— and —CH₂CH₂CH₂—. “Alkenyl” means carbon chains which contain at least one carbon-carbon double bond, and which may be linear or branched or combinations thereof such that C₂₋₆alkenyl is defined to identify the group as having 2, 3, 4, 5 or 6 carbons which incorporates at least one double bond, which may be in a E- or a Z-arrangement, including vinyl, allyl, isopropenyl, pentenyl, hexenyl, heptenyl, 1-propenyl, 2-butenyl, 2-methyl-2-butenyl, and the like. “Alkynyl” means carbon chains which contain at least one carbon-carbon triple bond, and which may be linear or branched or combinations thereof, such as ethynyl, propargyl, 3-methyl-1-pentynyl, 2-heptynyl and the like. “Cycloalkyl” means mono-, bi- or tri-cyclic structures, optionally combined with linear or branched structures, having the indicated number of carbon atoms, such as cyclopropyl, cyclopentyl, cycloheptyl, adamantyl, cyclododecylmethyl, 2-ethyl-1-bicyclo[4.4.0]decyl, and the like. “Alkoxy” means alkoxy groups of a straight or branched having the indicated number of carbon atoms. C₁₋₆alkoxy, for example, includes methoxy, ethoxy, propoxy, isopropoxy, and the like. “Heteroaryl” means mono- or bicyclic aromatic rings with at least one ring containing a heteroatom selected from N, O and S, and each ring containing 5 or 6 atoms. Examples of heteroaryl include benzoimidazolyl, benzimidazolonyl, benzofuranyl, benzofurazanyl, benzopyrazolyl, benzothiazolyl, benzotriazolyl, benzothiophenyl, benzoxazepin, benzoxazolyl, carbazolyl, carbolinyl, cinnolinyl, furanyl, furo(2,3-b)pyridyl, imidazolyl, indolinyl, indolyl, dihydroindolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydroquinoxalinyl, tetrazolyl, tetrazolopyridyl, thiadiazolyl, thiazolyl, thienyl, triazolyl, and N-oxides thereof, and the like.

A group which is designated as being independently substituted with substituents may be independently substituted with multiple numbers of such substituents.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

The present invention also includes all pharmaceutically acceptable isotopic variations of a compound of the Formula I in which one or more atoms is replaced by atoms having the same atomic number, but an atomic mass or mass number different from the atomic mass or mass number usually found in nature. Examples of isotopes suitable for inclusion in the compounds of the invention include isotopes of hydrogen such as 2H and 3H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulfur such as ³⁵S, fluorine such as ¹⁸F, iodine such as ¹²³I and ¹²⁵I, and chlorine such as ³⁶Cl. Certain isotopically-labelled compounds of Formula I, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of Formula I can generally be prepared by conventional techniques known to those skilled in the art or by processes analogous to those described in the accompanying Examples using appropriate isotopically-labelled reagents in place of the non-labelled reagent previously employed.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particular embodiments include the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particular embodiments citric, hydrobromic, hydrochloric, maleic, phosphoric, sulfuric, fumaric, and tartaric acids. It will be understood that, as used herein, references to the compounds of the present invention are meant to also include the pharmaceutically acceptable salts.

Exemplifying the invention are the specific compounds disclosed in the Examples and herein. The subject compounds are useful in a method of enhancing the neuromodulatory effect of metabotropic glutamate receptor activity in a patient such as a mammal in need of such enhancement comprising the administration of an effective amount of the compound. The present invention is directed to the use of the subject compounds disclosed herein as positive allosteric modulators of metabotropic glutamate receptor activity.

The invention also encompasses a pharmaceutical composition comprising a compound of Formula I in combination with a pharmaceutically acceptable carrier.

The invention also encompasses a method for treating a neurological or psychiatric disorder associated with glutamate dysfunction in a patient in need thereof comprising administering to the patient a therapeutically effective amount of a compound of Formula I. The invention also encompasses this method wherein the neurological or psychiatric disorder associated with glutamate dysfunction is schizophrenia.

The compounds of the present invention are modulators of metabotropic glutamate (mGluR) receptor function, in particular they are positive allosteric modulators of mGluR5 receptors. That is, the compounds of Formula I do not appear to bind to the orthosteric glutamate recognition site, and do not activate the mGluR5 by themselves. Instead, the response of mGluR5 to a concentration of glutamate or mGluR5 agonist is increased when a compound of Formula I is present. The compounds of Formula I are expected to have their effect at mGluR5 by virtue of their ability to enhance the function of the receptor. It is recognized that the compounds of the present invention would be expected to increase the effectiveness of glutamate and glutamate agonists of the mGluR5 receptor. Thus, the compounds of the present invention are expected to be useful in the treatment of various neurological and psychiatric disorders associated with glutamate dysfunction described to be treated herein and others that can be treated by such positive allosteric modulators as are appreciated by those skilled in the art.

The present invention is directed to the use of the compounds disclosed herein as positive allosteric modulators of mGluR5 receptor activity. The present invention is directed to a compound of the present invention or a pharmaceutically acceptable salt thereof for use in medicine. The present invention is further directed to a use of a compound of the present invention or a pharmaceutically acceptable salt thereof for the manufacture of a medicament for positive allosteric modulatorion of mGluR5 receptor activity or treating the disorders and diseases noted herein in humans and animals.

The present invention is further directed to a method for the manufacture of a medicament for positive allosteric modulation of metabotropic glutamate receptor activity in humans and animals comprising combining a compound of the present invention with a pharmaceutical carrier or diluent.

The subject treated in the present methods is generally a mammal, preferably a human being, male or female, in whom potentiation of metabotropic glutamate receptor activity is desired. In addition to primates, especially humans, a variety of other mammals can be treated according to the method of the present invention. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with the disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as positive allosteric modulators of metabotropic glutamate receptor activity, in particular mGluR5 activity, may be readily determined without undue experimentation by methodology well known in the art, including O'Brien et al., Molecular Pharmacology 2003, 64(3) 731-740. In particular, the compounds of the following examples had activity in reference assays by enhancing mGluR5 activity. The utility of the compounds as modulators of metabotropic glutamate receptor 5 (mGluR5) activation was demonstrated by their ability to increase an intracellular calcium flux above that achieved by a sub-threshold level of natural agonist (glutamate). Changes in intracellular Ca²⁺ were measured with Fluo-4AM ester (Invitrogen/Molecular Probes), which was detected on a Fluorometric Imaging Plate Reader (FLIPR, Molecular Devices, Sunnyvale, Calif.). In a typical experiment the mGluR5 positive allosteric modulatory activity of the compounds of the present invention was determined in accordance with the following experimental method.

Cell Culture:

Chinese Hamster Ovary (CHO) cells expressing human mGluR5_{A} were maintained in growth medium containing DMEM, 10% dialyzed Fetal Bovine Serum, 50 units/mL Penicillin, 50 ug/mL Streptomycin, 2 mM L-glutamine, 1×MEM non-essential amino acids, 1 mM sodium pyruvate, 25 mM HEPES, 55 uM 2-mercaptoethanol, 5 ug/mL Puromycin, and 250 ug/mL Zeocin at 37° C. and 5% CO₂. The day before the experiment, the cells were washed and seeded in “plating media” containing only DMEM, 10% dialyzed Fetal Bovine Serum, 50 units/mL Penicillin, and 50 ug/mL Streptomycin at a density of 50,000 cells/well (100 uL/well) in black 384-well clear-bottom PDL-coated plates. The cells were grown overnight at 37° C. and 6% CO₂. This overnight glutamine/glutamate starvation allowed for consistent expression of the mGluR5_{A} receptor, and the ability to add a known amount of agonist (glutamate in most cases) on the day of the experiment.

Fluorescent Ca²⁺ Mobilization (FLIPR) Assay:

The day of the experiment, the cells were washed with 37° C. Assay Buffer (Hanks Balanced Salt Solution with CaCl₂ and MgCl₂, 20 mM HEPES, 2.5 mM Probenecid, 0.1% BSA) with an automated plate washer (3×100 uL, aspiration 3 mm from bottom leaving ˜30 uL of buffer in each well). After washing, 30 uL of dye loading buffer (4 uM Fluo-4AM, 0.04% Pluronic acid, and 1% dialyzed FBS in assay buffer) were added to each well of the plates for 2 uM Fluo-4AM final concentration. The plates were incubated at 37° C. and 6% CO₂ for 1 hour to allow for dye loading. After dye loading, the cells were washed again as above, and placed on the FLIPR. Assays were conducted with two possible scenarios: 1) To determine the potencies of the compounds, as either agonists of mGluR5 or potentiators of mGluR5 in the presence of a sub-threshold amount of glutamate, 10-point titrations of the compounds (1:3 dilution between each point, 30-0.0015 uM final concentrations) were added to the cells, followed by the addition of the EC₂₀ of glutamate (300 nM) to the cells. 2) To determine the cooperativity of the compounds with the natural agonist (glutamate), single concentrations of the compounds were added to the cells, followed by the addition of a 10-point titration of glutamate (1:3 dilution between each point, 1000-0.05 uM final concentrations). When compared to the EC₅₀ of glutamate in the presence of DMSO only on the same assay plate, a left-shift in the glutamate dose-response curve in the presence of compound demonstrates the degree of potentiation at the single concentration of the compound. For both scenarios above, operation of the FLIPR was the same. Baseline fluorescence was monitored for 10 seconds, followed by the addition of compounds diluted in Assay Buffer (1% DMSO concentration after this addition, 0.66% final DMSO concentration after agonist addition). After monitoring fluorescence for 5 minutes, during which time any intrinsic agonist activity of the compounds would have been detected, the agonist (glutamate) also diluted in assay buffer was then added to the cells. The response was then monitored for an additional 3 minutes. In scenario #1, the peak during the final 3 minutes was used for potentiator data, and the peak during the 5 minutes post compound addition was used for compound agonist data. Inflection points for potentiation and agonism were determined with non-linear curve fitting, and the maximal response of the compound was compared to the maximal response of the agonist (1 mM glutamate) to provide a % of max activity for each compound. Additionally, the maximal response of each compound was compared to the sub-threshold response of the agonist (300 nM glutamate) to provide a fold potentiation value at the maximal response.

Potencies for the compounds are reported as EC₅₀ values for agonism (in the absence of 300 nM glutamate) “EC₅₀ values” (actually inflection points) for potentiation (in the presence of 300 nM glutamate).

% Max_{—}1 mM glutamate=fluorescence counts caused by compound/fluorescence counts caused by 1 mM glutamate×100%

fold potentiation=fluorescence counts caused by compound/fluorescence counts caused by 300 nM glutamate

In scenario #2, the peak during the final 3 minutes was used for the points of the agonist dose response curve. The EC₅₀ values for the agonist in the presence of 0.66% DMSO or each single concentration of the compound were determined with non-linear curve fitting. By dividing the EC₅₀ of glutamate+DMSO by the EC₅₀ of glutamate+compound, the resulting value is the fold-shift in agonist potency, and therefore the degree of potentiation of the compound at the given concentration. This value is called the “glutamate shift”

$$Glutamate shift = \frac{{EC}_{50} of glutamate in the presence of 0.66 \% DMSO}{\begin{matrix}{EC}_{50} of glutamate in the presence of \\ a given concentration of compound\end{matrix}}$$

The compounds of the following Examples were tested and had activity as positive allosteric modulators of the mGluR5 receptor in the foregoing assays. In particular, the compounds of Examples 1-5, 1-6, 2-1, 3-1, 4-4, 4-5, 5-3, 5-4, 6-15, 6-16, 7-7, 10-8, 11-7, 12-6, 12-7, 13-2, 14-9, 14-10, 15-1, 15-2, 16-1, 16-2, 17-3, 17-4, 18-1, 19-2, 20-3, 21-2, 22-4, 23-17, 24-6, 24-7, 25-3, 25-4, 26-9, 26-10, 26-13, 27-1, 27-2, 28-2, 28-3, 30-6, 31-3, 31-4, 32-1, 32-2, 32-3, 32-4, 32-5, 32-6, 32-7, 32-8, 32-9, 32-10, 32-11, 32-12, 32-13, 32-14, 32-15, 32-16, 32-17, 32-18, 32-19, and 32-20 had activity in potentiating the mGluR5 receptor in the FLIPR assay with an EC₅₀ of about 0.001 μM to 10 μM. For example, the compounds of Examples 1-5, 3-1, 7-7, 11-8, 13-2, 18-1, 20-3, 24-6 were tested and demonstrated activity in enhancing the mGluR5 receptor in the FLIPR assay, generally with an EC₅₀ of less than about 1 μM. The compounds of Examples 1-5, 3-1, 20-3 and 24-6 exhibited a glutamate shift of at least 4× at concentrations up to 30 μM. Such results are indicative of the intrinsic activity of the compounds for use as potentiators of mGluR5 receptor activity. For a compound to have therapeutic utility, it is expected that such compound should have activity in enhancing the mGluR5 receptor in the FLIPR assay with an EC₅₀ of less than about 10 μM.

TABLE 1
Representative FLIPR EC₅₀ Values

| Example | EC50 |
|---|---|
| 1-5 | 16 nM |
| 3-1 | 8 nM |
| 7-7 | 31 nM |
| 11-8 | 71 nM |
| 18-1 | 50 nM |

Metabotropic glutamate receptors including the mGluR5 receptor have been implicated in a wide range of biological functions. This has suggested a potential role for these receptors in a variety of disease processes in humans or other species. See e.g., Byrnes, et al., Neurotherapeutics, 6, 94-107 (2009). The compounds of the present invention have utility in treating, preventing, ameliorating, controlling or reducing the risk of a variety of neurological and psychiatric disorders associated with glutamate dysfunction, including one or more of the following conditions or diseases: schizophrenia or psychosis including schizophrenia (paranoid, disorganized, catatonic, undifferentiated, or residual type), schizophreniform disorder, schizoaffective disorder, for example of the delusional type or the depressive type, delusional disorder, psychotic disorder, brief psychotic disorder, shared psychotic disorder, psychotic disorder due to a general medical condition and substance-induced or drug-induced (for example psychosis induced by alcohol, amphetamine, cannabis, cocaine, hallucinogens, inhalants, opioids, phencyclidine, ketamine and other dissociative anaesthetics, and other psychostimulants), psychosispsychotic disorder, psychosis associated with affective disorders, brief reactive psychosis, schizoaffective psychosis, “schizophrenia-spectrum” disorders such as schizoid or schizotypal personality disorders, personality disorder of the paranoid type, personality disorder of the schizoid type, illness associated with psychosis (such as major depression, manic depressive (bipolar) disorder, Alzheimer's disease and post-traumatic stress syndrome), including both the positive and the negative symptoms of schizophrenia and other psychoses; disorders that comprise as a symptom a deficiency in attention and/or cognition; cognitive disorders including dementia (associated with Alzheimer's disease, ischemia, multi-infarct dementia, trauma, intracranial tumors, cerebral trauma, vascular problems or stroke, alcoholic dementia or other drug-related dementia, AIDS, HIV disease, Parkinson's disease, Huntington's disease, Pick's disease, Creutzfeldt-Jacob disease, perinatal hypoxia, other general medical conditions or substance abuse); Alzheimer's disease, multi-infarct dementia, AIDS-related dementia, and Fronto temperal dementia; delirium, amnestic disorders or age related cognitive decline; migraine, migraine headache; pain including acute pain, chronic pain, severe pain, intractable pain, neuropathic pain, post-traumatic pain, bone and joint pain (osteoarthritis), repetitive motion pain, dental pain, cancer pain, myofascial pain (muscular injury, fibromyalgia), perioperative pain (general surgery, gynecological), chronic pain, neuropathic pain; trigeminal neuralgia; amyotrophic lateral sclerosis (ALS); cerebral deficits subsequent to cardiac bypass surgery and grafting, stroke, cerebral ischemia, spinal cord trauma, head trauma, perinatal hypoxia, cardiac arrest, hypoglycemic neuronal damage; spinal cord injury; neuronal regeneration; neuronal inflammation; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder, drug addiction, tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders, major depressive episode of the mild, moderate or severe type, a manic or mixed mood episode, a hypomanic mood episode, a depressive episode with atypical features, a depressive episode with melancholic features, a depressive episode with catatonic features, a mood episode with postpartum onset, post-stroke depression; major depressive disorder, dysthymic disorder, minor depressive disorder, premenstrual dysphoric disorder, post-psychotic depressive disorder of schizophrenia, a major depressive disorder superimposed on a psychotic disorder such as delusional disorder or schizophrenia, a bipolar disorder, for example bipolar I disorder, bipolar II disorder, cyclothymic disorder, depression including unipolar depression, seasonal depression and post-partum depression, premenstrual syndrome (PMS) and premenstrual dysphoric disorder (PDD), mood disorders due to a general medical condition, and substance-induced mood disorders; learning disorders, for example reading disorder, mathematics disorder, or a disorder of written expression, attention-deficit/hyperactivity disorder, and age-related cognitive decline, pervasive developmental disorder including autistic disorder, attention disorders including attention-deficit hyperactivity disorder (ADHD) and conduct disorder; NMDA receptor-related disorders such as autism, depression, benign forgeffulness, childhood learning disorders and closed head injury; neurodegenerative disorders or conditions, neurodegeneration associated with cerebral trauma; neurodegeneration associated with stroke, neurodegeneration associated with cerebral infarct, hypoglycemia-induced neurodegeneration, neurodegeneration associated with epileptic seizure, neurodegeneration associated with neurotoxin poisoning, multi-system atrophy; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Huntington's disease, dyskinesia associated with dopamine agonist therapy, Gilles de la Tourette's syndrome, epilepsy, muscular spasms and disorders associated with muscular spasticity or weakness including tremors; dyskinesias, including tremor (such as rest tremor, postural tremor, intention tremor and essential tremor), tardive dyskinesia, restless leg syndrome, chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

Among the disorders above, of particular importance are the treatment of schizophrenia, migraine, anxiety (including agoraphobia, generalized anxiety disorder (GAD), obsessive-compulsive disorder (OCD), panic disorder, posttraumatic stress disorder (PTSD), social phobia, other phobias, substance-induced anxiety disorder), mood disorders (including bipolar disorders (I & II), cyclothymic disorder, depression, dysthymic disorder, major depressive disorder, substance-induced mood disorder), attention-deficit/hyperactivity disorder (ADD, ADHD), eating disorders (including anorexia nervosa, bulimia nervosa), epilepsy, cognitive disorders (including delirium, substance-induced persisting delirium, dementia, dementia due to HIV disease, dementia due to Huntington's disease, dementia due to Parkinson's disease, dementia of the Alzheimer's type, substance-induced persisting dementia, mild cognitive impairment), personality disorders (including obsessive-compulsive personality disorder, schizoid, schizotypal disorder), substance-related disorders (including alcohol abuse, alcohol dependence, alcohol withdrawal, alcohol withdrawal delirium, alcohol-induced psychotic disorder, amphetamine dependence, amphetamine withdrawal, cocaine dependence, cocaine withdrawal, nicotine dependence, nicotine withdrawal, opioid dependence, opioid withdrawal).

In another specific embodiment, the present invention provides a method for treating schizophrenia or psychosis comprising: administering to a patient in need thereof an effective amount of a compound of the present invention. Particular schizophrenia or psychosis pathologies are paranoid, disorganized, catatonic or undifferentiated schizophrenia and substance-induced psychotic disorder. At present, the text revision of the fourth edition of the Diagnostic and Statistical Manual of Mental Disorders (DSM-IV-TR) (2000, American Psychiatric Association, Washington D.C.) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

Thus, in an embodiment the present invention provides a method for treating migraine, comprising: administering to a patient in need thereof an effective amount of a compound of the present invention. In one of the available sources of diagnostic tools, Dorland's Medical Dictionary (23'd Ed., 1982, W.B. Saunders Company, Philadelphia, Pa.), migraine is defined as a symptom complex of periodic headaches, usually temporal and unilateral, often with irritability, nausea, vomiting, constipation or diarrhea, and photophobia. As used herein the term “migraine” includes these periodic headaches, both temporal and unilateral, the associated irritability, nausea, vomiting, constipation or diarrhea, photophobia, and other associated symptoms. The skilled artisan will recognize that there are alternative nomenclatures, nosologies, and classification systems for neurological and psychiatric disorders, including migraine, and that these systems evolve with medical scientific progress.

In another specific embodiment, the present invention provides a method for treating anxiety disorders, comprising: administering to a patient in need thereof an effective amount of a compound of the present invention. Particular anxiety disorders are generalized anxiety disorder, obsessive-compulsive disorder and panic attack. At present, the text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In another embodiment the present invention provides a method for treating depression, comprising: administering to a patient in need thereof an effective amount of a compound of the present invention. At present, the fourth edition of the Diagnostic and Statistical Manual of Mental Disorders (DSM-IV) (1994, American Psychiatric Association, Washington, D.C.), provides a diagnostic tool including depression and related disorders. Depressive disorders include, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder. As used herein the term “depression” includes treatment of those depression disorders and related disorder as described in the DSM-IV.

In another embodiment the present invention provides a method for treating epilepsy, comprising: administering to a patient in need thereof an effective amount of a compound of the present invention. At present, there are several types and subtypes of seizures associated with epilepsy, including idiopathic, symptomatic, and cryptogenic. These epileptic seizures can be focal (partial) or generalized. They can also be simple or complex. Epilepsy is described in the art, such as Epilepsy: A comprehensive textbook. Ed. by Jerome Engel, Jr. and Timothy A. Pedley. (Lippincott-Raven, Philadelphia, 1997). At present, the International Classification of Diseases, Ninth Revision, (ICD-9) provides a diagnostic tool including epilepsy and related disorders. These include: generalized nonconvulsive epilepsy, generalized convulsive epilepsy, petit mal status epilepticus, grand mal status epilepticus, partial epilepsy with impairment of consciousness, partial epilepsy without impairment of consciousness, infantile spasms, epilepsy partialis continua, other forms of epilepsy, epilepsy, unspecified, NOS. As used herein the term “epilepsy” includes these all types and subtypes. The skilled artisan will recognize that there are alternative nomenclatures, nosologies, and classification systems for neurological and psychiatric disorders, including epilepsy, and that these systems evolve with medical scientific progress.

In a specific embodiment, the present invention provides a method for treating cognitive disorders, comprising: administering to a patient in need thereof an effective amount of a compound of the present invention. Particular cognitive disorders are dementia, delirium, amnestic disorders and age-related cognitive decline. At present, the text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In another specific embodiment, the present invention provides a method for treating substance-related disorders and addictive behaviors, comprising: administering to a patient in need thereof an effective amount of a compound of the present invention. Particular substance-related disorders and addictive behaviors are persisting dementia, persisting amnestic disorder, psychotic disorder or anxiety disorder induced by substance abuse; and tolerance of, dependence on or withdrawal from substances of abuse. At present, the text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

In another specific embodiment, the present invention provides a method for treating pain, comprising: administering to a patient in need thereof an effective amount of a compound of the present invention. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

The subject compounds are further useful in a method for the prevention, treatment, control, amelioration, or reduction of risk of the diseases, disorders and conditions noted herein.

The subject compounds are further useful in a method for the prevention, treatment, control, amelioration, or reduction of risk of the aforementioned diseases, disorders and conditions in combination with other agents, including an mGluR agonist.

The subject compounds are further useful in a method for the prevention, treatment, control, amelioration, or reduction of risk of the diseases, disorders and conditions noted herein. The subject compounds are further useful in a method for the prevention, treatment, control, amelioration, or reduction of risk of the aforementioned diseases, disorders and conditions in combination with other agents. The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and the compound of the present invention may be desirable. However, the combination therapy may also includes therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention. The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. Accordingly, the pharmaceutical compositions of the present invention include those that also contain one or more other active ingredients, in addition to a compound of the present invention. The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, such as about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination.

In one embodiment, the subject compound may be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies.

In another embodiment, the subject compound may be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, atypical antipsychotics, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexyl)hydrochloride, COMT inhibitors such as entacapone, MOA-B inhibitors, antioxidants, Ata adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound may be employed in combination with acetophenazine, alentemol, aripiprazole, amisulpride, benzhexyl, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The pharmaceutical composition and method of the present invention may further comprise other therapeutically active compounds as noted herein which are usually applied in the treatment of the above mentioned pathological conditions.

The subject compounds are further useful in a method for the prevention, treatment, control, amelioration, or reduction of risk of the diseases, disorders and conditions noted herein. The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 30 mg/kg. of body weight daily are administered to the patient, e.g., humans and elderly humans. The dosage range will generally be about 0.5 mg to 5.0 g. per patient per day which may be administered in single or multiple doses. In one embodiment, the dosage range will be about 0.5 mg to 2.5 mg per patient per day; in another embodiment about 0.5 mg to 1 g per patient per day; in yet another embodiment about 5 mg to 500 mg per patient per day; and in yet another embodiment about 5 mg to 100 mg per patient per day. Pharmaceutical compositions of the present invention may be provided in a solid dosage formulation such as comprising about 0.5 mg to 800 mg active ingredient, or comprising about 1 mg to 400 mg active ingredient. The pharmaceutical composition may be provided in a solid dosage formulation comprising about 1 mg, 5 mg, 10 mg, 25 mg, 50 mg, 100 mg, 200 mg or 250 mg active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, such as once or twice per day.

When treating, preventing, controlling, ameliorating, or reducing the risk of neurological and psychiatric disorders associated with glutamate dysfunction or other diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 5000 milligrams, preferably from about 1 milligrams to about 1000 milligrams. In the case of a 70 kg adult human, the total daily dose will generally be from about 7 milligrams to about 800 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials and the requisite intermediates are in some cases commercially available, or can be prepared according to literature procedures or as illustrated herein. The compounds of this invention may be prepared by employing reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are prepared by employing reactions as shown in the schemes and examples herein, in addition to other standard manipulations such as ester hydrolysis, cleavage of protecting groups, etc., as may be known in the literature or exemplified in the experimental procedures. Starting materials are made according to procedures known in the art or as illustrated herein. The following abbreviations are used herein: Me: methyl; Et: ethyl; t-Bu: tert-butyl; Ar: aryl; Ph: phenyl; Bn: benzyl; Ac: acetyl; THF: tetrahydrofuran; DIEA: N,N-diisopropylethylamine; DMSO: dimethylsulfoxide; EDC: N-(3-dimethylaminopropyl)-N′-ethylcarbodiimide hydrochloride; HOBT: hydroxybenzotriazole hydrate; Boc: Cert-butyloxy carbonyl; Et₃N: triethylamine; EtOAc: ethyl acetate; CH₂Cl₂: dichloromethane; CH₃OH: methanol; C₂H₅OH: ethanol; CH₃CN: acetonitrile; BSA: bovine serum albumin; TFA: trifluoracetic acid; DMF: N,N-dimethylformamide; MTBE: methyl tert-butyl ether; SOCl₂: thionyl chloride; CDI: carbonyl diimidazole; RT: room temperature; HPLC: high performance liquid chromatography; TEMPO: 2,2,6,6,-tetramethyl-1-piperidine 1-oxyl; HATU: O-(7-azabenzotriazol-1-yl)-N,N,′,′-tetramethyluronium hexafluorophosphate; Burgess reagent: methoxycarbonylsulfamoyl) trimethylammonium inner salt. The compounds of the present invention can be prepared in a variety of fashions.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### Intermediates A-2 Through A-7

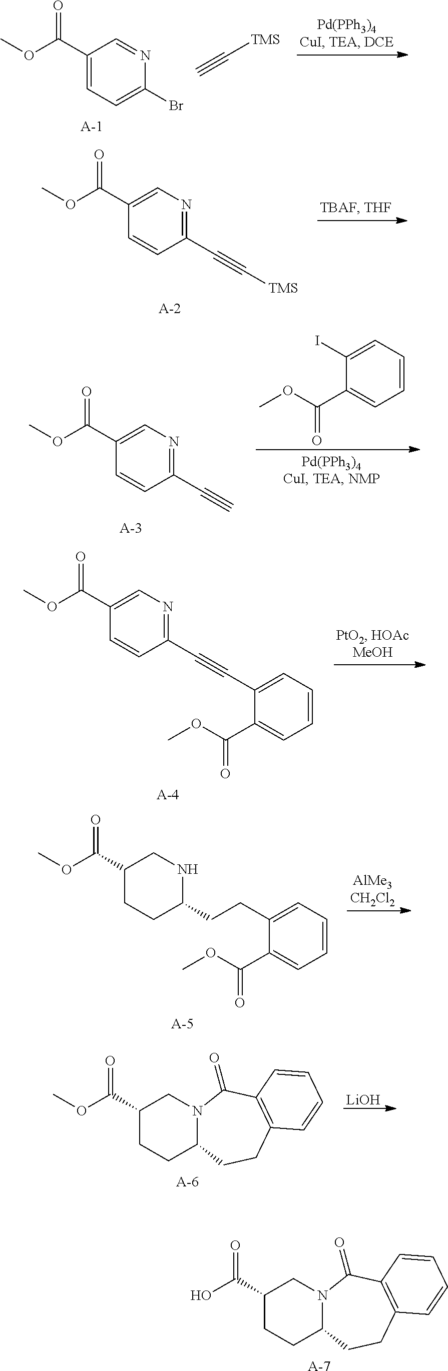

### Methyl 6-((trimethylsilyl)ethynyl)nicotinate (A-2)

Methyl 6-bromonicotinate A-1 (28.25 g, 131 mmol) was dissolved in dichloroethane (150 mL) and cooled to 0° C. To this solution was added TEA (80 mL), trimethylsilyl acetylene (24 mL, 171 mmol), bis(triphenylphosphine)palladium (II) chloride (1.2 g, 1.71 mmol) and CuI (0.6 g, 3.15 mmol). The reaction was allowed to warm to RT and stirred overnight. The reaction mixture was diluted with hexane (100 mL). After filtration, the organic solution was washed with water and brine. The organic layer was concentrated and chromatographed on silica gel (330 g column, 5 to 10% EtOAc in heptane) to give A-2. MS m/z (M+H) 234.0 found, 234.3 required.

### Methyl 6-ethynylpyridine-3-carboxylate (A-3)

Methyl 6-((trimethylsilyl)ethynyl)nicotinate A-2 (27.1 g, 116 mmol) was dissolved in THF (111 mL) and cooled over ice bath. To this cold solution was added a solution of TBAF (1M in THF, 139 mL, 139 mmol) and the solution was removed from the cooling bath and allowed to stir at 25° C. for 20 min. The reaction mixture was then concentrated in vacuo and the residue partitioned between ethyl acetate and aq. sodium bicarbonate. The organic extract was filtered through celite and the solvent removed in vacuo. This residue was chromatographed on silica gel (0-30% ethyl acetate/hexanes) to give A-3 as an oil. MS m/z (M+H) 161.9 found, 162.1 required.

### Methyl 6-{[2-(methoxycarbonyl)phenyl]ethynyl}pyridine-3-carboxylate (A-4)

Methyl 6-ethynylpyridine-3-carboxylate A-3 (11.7 g, 72.6 mmol) and methyl 2-iodobenzoate (11.73 mL, 80 mmol) were combined in dichloroethane (105 mL) and TEA (70 mL) and degassed. This solution was treated with palladium (II) tetrakis triphenylphosphine (4.19 g, 3.63 mmol) and CuI (1.106 g, 5.81 mmol) and heated to 50° C. for 1 hour. Stirred at 25° C. for 18 hours and then removed the solvent in vacuo. This residue was purified by chromatography on silica gel (0-20% ethyl acetate/DCM) to give title compound. MS m/z (M+H) 296.0 found, 296.3 required.

### (±) Methyl (3S,6S)-6-{2-[2-(methoxycarbonyl)phenyl]ethyl}piperidine-3-carboxylate (A-5)

Methyl 6-{[2-(methoxycarbonyl)phenyl]ethynyl}pyridine-3-carboxylate A-4 (4.0 g, 13.5 mmol) was dissolved in MeOH (40 mL) with AcOH (772 uL, 13.5 mmol) and shaken with PtO₂H₂O (2.0 g, 8.16 mmol) over hydrogen (45 psi) for 4 days. After this time, filtered the mixture through celite and washed the pad with MeOH (50 mL) and AcOH (40 mL). The combined filtrate was concentrated in vacuo and the residue was partitioned between aq. sat. NaHCO₃ and DCM (3×150 mL). The combined extracts were purified by chromatography on silica gel (120 g, 0-10% aq. NH₄OH/CAN to give A-5. MS m/z (M+H) 306.5 found, 306.4 required.

### (±) Methyl-(cis)-6-oxo-1,2,3,4,6,11,12,12a-octahydropyrido[1,2-b][2]benzazepine-3-carboxylate (A-6)

To a solution of A-5 (3.64 g, 11.9 mmol) in DCM (140 mL) cooled over a dry ice/acetone bath was added trimethylaluminum (2M in toluene, 8.94 mL, 17.9 mmol). After addition was complete, the cooling bath was removed and the reaction was stirred at 25° C. for 18 hours. After this time, the reaction mixture was quenched with crushed ice (50 g) and made acidic with 1N HCl (100 mL) and extracted with DCM (300+250 mL). The combined extracts were dried over MgSO₄, filtered and the solvent removed in vacuo. This residue was chromatographed on silica gel (120, 0-20% EtOAc/DCM) to give A-6. MS m/z (M+H) 274.5 found, 274.3 required.

### (±) Cis-6-oxo-1,2,3,4,6,11,12,12a-octahydropyrido[1,2-b][2]benzazepine-3-carboxylic acid (A-7)

To a solution of A-6 (1.95 g, 7.13 mmol) in MeOH (40 mL) was added 1.0N LiOH (14.27 mL, 14.27 mmol) and stirred at 25° C. for 30 min. After this time, concentrated (15 mL volume) in vacuo and treated with 1N HCl (20 mL, 20 mmol) and then extracted with DCM (2×100 mL). The combined extracts were dried over MgSO₄, filtered and the solvent removed in vacuo to give A-7. MS m/z (M+H) 260.5 found, 260.3 required.

### Intermediates B-1 Through B-3

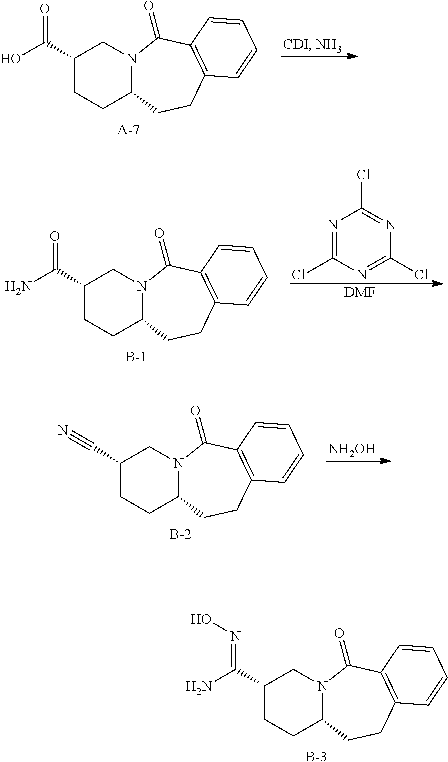

### (±) Cis-6-oxo-1,2,3,4,6,11,12,12a-octahydropyrido[1,2-b][2]benzazepine-3-carboxamide (B-1)

To a suspension of A-7 (1.69 g, 6.52 mmol) in ACN (20 mL) was added carbonyl diimidazole (1.585 g, 9.78 mmol) and stirred for 20 min as the suspension dissolves. This solution was treated with sat. aq. ammonium hydroxide (6.6 mL, 98 mmol) and stirred for 20 min. This mixture was partitioned between EtOAc (200 mL) and 1N HCl (200 mL). The extract was washed with brine, dried over MgSO₄, filtered and the solvent removed in vacuo to give B-1 as a solid. MS m/z (M+H) 259.5 found, 259.3 required.

### (±) Cis-6-oxo-1,2,3,4,6,11,12,12a-octahydropyrido[1,2-b][2]benzazepine-3-carbonitrile (B-2)

To a solution of 13-1 (1.657 g, 6.41 mmol) in DMF (35 mL) cooled in an ice bath was added cyanuric chloride (887 mg, 4.81 mmol). This was stirred for 5 min and then the cooling bath was removed and stirring continued an additional 20 min. After this time, LC-MS showed incomplete reaction. The reaction was recooled over ice bath and additional cyanuric chloride (887 mg, 4.81 mmol) was added. The reaction was stirred in the cold for 5 min and then the cooling bath was removed and the reaction stirred an additional 20 min. The reaction mixture was partitioned between water (100 mL) and EtOAc (2×100 mL). The combined extracts were washed with water (20 mL) and brine (20 mL), dried over MgSO₄, filtered and the solvent removed in vacuo. This residue was chromatographed on silica gel (40 g, 0-5% EtOAc/DCM) to give B-2 as a solid. MS m/z (M+H) 241.5 found, 241.3 required.

### (±) cis-N′-hydroxy-6-oxo-1,2,3,4,6,11,12,12a-octahydropyrido[1,2-b][2]-benzazepine-3-carboximidamide (B-3)

To a suspension of 13-2 (1.0 g, 4.16 mmol) in ethanol (14 mL) was added hydroxylamine hydrochloride (304 mg, 4.37 mmol) and TEA (609 uL, 4.37 mmol) and heated to 80° C. for 3 hours. After this time, the solvent was removed in vacuo and then further azeotroped with toluene (20 mL) to give B-3 along with Et₃N.HCl as a solid. MS m/z (M+H) 274.5 found, 274.3 required.

### Intermediates C-1 Through C-3

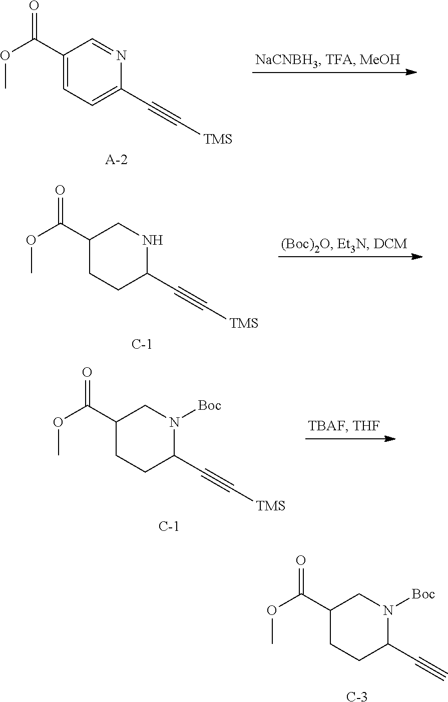

### (±) Methyl 6-((trimethylsilyl)ethynyl)piperidine-3-carboxylate (C-1)

Intermediate A-2 (7.0 g, 30.0 mmol) was dissolved in methanol (110 mL) and cooled to 0° C. To this solution was added TFA (16.0 mL, 181 mmol) and NaCNBH₃ in five portions (10.0 g, 159 mmol) during a period of one hour. The reaction was allowed to warm to RT and stirred for one hour. To this reaction mixture was added sat NaHCO₃ (200 mL) and EtOAc (300 mL). The aqueous phase was extracted with EtOAc and combined organic phase was washed with brine and concentrated to give crude C-1 as a mixture of stereoisomers. MS m/z (M+H) 240.0 found, 240.3 required.

### (±) 1-tert-Butyl 3-methyl 6-((trimethylsilyl)ethynyl)piperidine-1,3-dicarboxylate (C-2)

Intermediate C-1 (7.2 g, crude, 30 mmol) was dissolved in DCM (120 mL) and cooled to 0° C. To this solution was added TEA (6.0 mL, 43 mmol), (Boc)₂O (7.9 g, 36 mmol) and DMAP (0.4 g, 3.27 mmol). The reaction was allowed to warm to RT and stirred overnight. The reaction solution was washed with water and concentrated. The organic residue was chromatographed on silica gel (120 g column, 0 to 10% EtOAc in heptane) to give C-2. MS m/z (M+H) 340.3 found, 340.5 required.

### (±) 1-tert-Butyl 3-methyl 6-ethynylpiperidine-1,3-dicarboxylate (C-3)

Intermediate C-2 (7.1 g, 20.9 mmol) was dissolved in THF (120 mL) and cooled to 0° C. To this solution was added 1M TBAF solution in THF (20.9 mL, 20.9 mmol). The reaction was allowed to warm to RT and stirred for 2 h. The reaction solution concentrated to about half of original volume and partitioned between EtOAc and water. The organic phase was washed with water and brine. The concentrated organic phase was chromatographed on silica gel (120 g column, 10 to 20% EtOAc in heptane) to give C-3. MS m/z (M+Na) 290.3 found, 290.3 required.

### Intermediates D-2 Through D-7

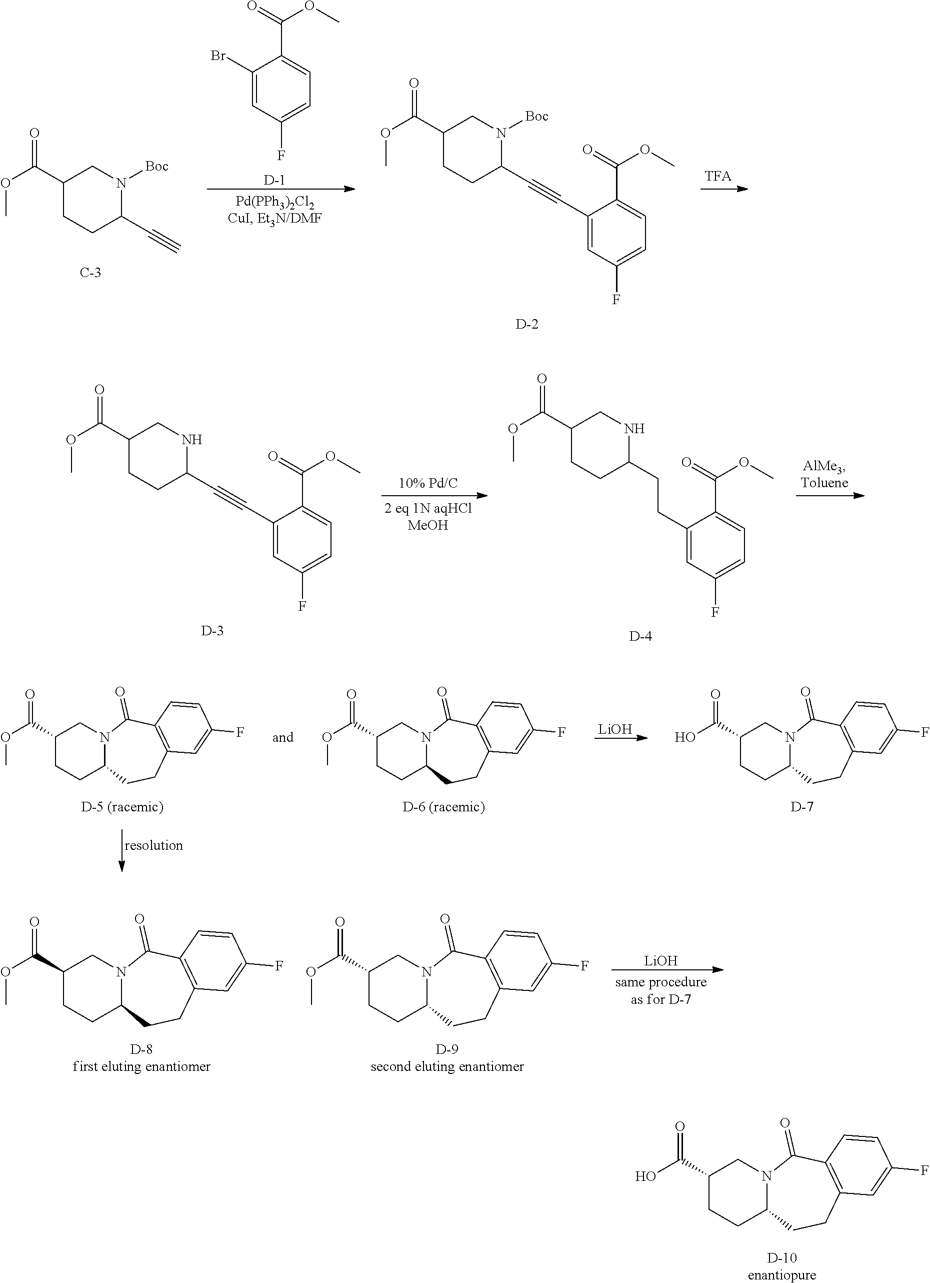

### (±) 1-tert-Butyl 3-methyl 6-((5-fluoro-2-(methoxycarbonyl)phenyl)ethynyl)piperidine-1,3-dicarboxylate (D-2)

To a suspension of bis(triphenylphosphine)palladium (H) chloride (9.33 g, 13.3 mmol) and CuI (2.53 g, 13.3 mmol) in DMF (500 mL) was added methyl-2-bromo-4-fluorobenzoate D-1 (38.7 g, 166 mmol) and TEA (255 mL). After stirred at RT for 10 minutes, the solution of intermediate C-3 (47 g, 176 mmol) in DMF (50 mL) was added. The reaction mixture was warmed to 35° C. and stirred overnight. The reaction was partitioned between EtOAc and water. The organic phase washed with brine and concentrated. The crude was purified by silica gel chromatography (1.5 kg column, 10-30% EtOAc in heptane) to give D-2. MS m/z (M+Na) 442.2 found, 442.4 required.

### (±) Methyl 6-((5-fluoro-2-(methoxycarbonyl)phenyl)ethynyl)piperidine-3-carboxylate (D-3)

Intermediate D-2 (58.4 g, 139 mmol) was dissolved in CH₂Cl₂ (200 mL) and cooled to 0° C. To this solution was added TFA (200 mL). The reaction was allowed to warm to RT and stirred for 30 min. The reaction was concentrated to give D-3. MS m/z (M+H) 320.3 found, 320.3 required.

### (±) Methyl 6-(5-fluoro-2-(methoxycarbonyl)phenethyl)piperidine-3-carboxylate (D-4)

Intermediate D-3 (139 mmol) was dissolved in MeOH (800 mL). To this solution was added 1N HCl (139 mL) and 10% Palladium on charcoal (17 g). The reaction vessel was put on hydrogenator at 55 psi for 2 h. The reaction mixture was filtered through celite and the solution was concentrated. The crude product D-4 was used directly in next step. MS m/z (M+H) 324.3 found, 324.4 required.

### (±) cis-Methyl 9-fluoro-6-oxo-1,2,3,4,6,11,12,12a-octahydrobenzo[e]pyrido[1,2-a]azepine-3-carboxylate (D-5)

A solution of intermediate D-4 (139 mmol) in CH₂Cl₂ (1.4 L) was cooled to 60° C. To this solution was added trimethylaluminium (2 M in toluene, 140 mL). The reaction solution was warmed to RT and stirred overnight. The reaction was slowly poured into cold 1N HCl (1 L). The organic layer was dried over Na₂SO₄ and concentrated. The crude product was purified by silica gel chromatography (1.5 kg column, 10-50% EtOAc in heptane) to give D-5 (cis racemate) and D-6 (trans racemate). MS m/z (M+H) 292.3 found, 292.3 required.

### (±) cis-9-Fluoro-6-oxo-1,2,3,4,6,11,12,12a-octahydrobenzo[e]pyrido[1,2-a]azepine-3-carboxylic acid (D-7)

To a solution of intermediate D-5 (9.9 g, 34 mmol) in THF (300 mL) was added aqueous LiOH solution (1N, 68 mL) at RT. The reaction solution was stirred for 2 h. Aqueous HCl (1N, 68 mL) was added to the reaction. The mixture was partitioned between CH₂Cl₂ and water. The organic layer was washed with brine and concentrated to give D-7. MS m/z (M+H) 278.3 found, 278.3 required.

### methyl (3R,12aR)-9-fluoro-6-oxo-1,2,3,4,6,11,12,12a-octahydropyrido[1,2-b][2]benzazepine-3-carboxylate (D-8) and methyl (3S,12aS)-9-fluoro-6-oxo-1,2,3,4,6,11,12,12a-octahydropyrido[1,2-b][2]benzazepine-3-carboxylate (D-9)

Enantiomerically pure compounds D-8 and D-9 were obtained from chiral HPLC separation of racemic D-5. Preparative HPLC was performed on AS-H column (3 cm×25 cm, eluted with 80% CO₂ and 20% MeOH at 2.4 mL/min flow rate at 35° C. D-8 (peak one): MS m/z (M+H) 292.3 found, 292.3 required. D-9 (peak two): MS m/z (M+H) 292.3 found, 292.3 required.

### Intermediate E-1

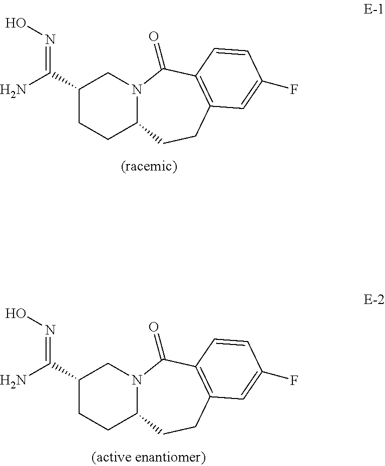 [SMILES: N/C(=N\O)[C@H]1CC[C@H]2CCc3cc(F)ccc3C(=O)N2C1 ; N/C(=N\O)[C@H]1CC[C@H]2CCc3cc(F)ccc3C(=O)N2C1]

### (±) cis-9-Fluoro-N′-hydroxy-6-oxo-1,2,3,4,6,11,12,12a-octahydrobenzo[e]pyrido[1,2-a]azepine-3-carboximidamide (E-1) and (3S,12aS)-9-fluoro-N′-hydroxy-6-oxo-1,2,3,4,6,11,12,12a-octahydropyrido[1,2-b][2]benzazepine-3-carboximidamide (E-2)

Intermediate E-1 was obtained following the same procedure described for intermediate B-3, but starting from intermediate D-7. m/z (M+H) 292.3 found, 292.3 required. The active enantiomer E-2 was synthesized following the same procedure as intermediate B-3, but starting from intermediate D-9.

### Intermediates F-2 Through F-8

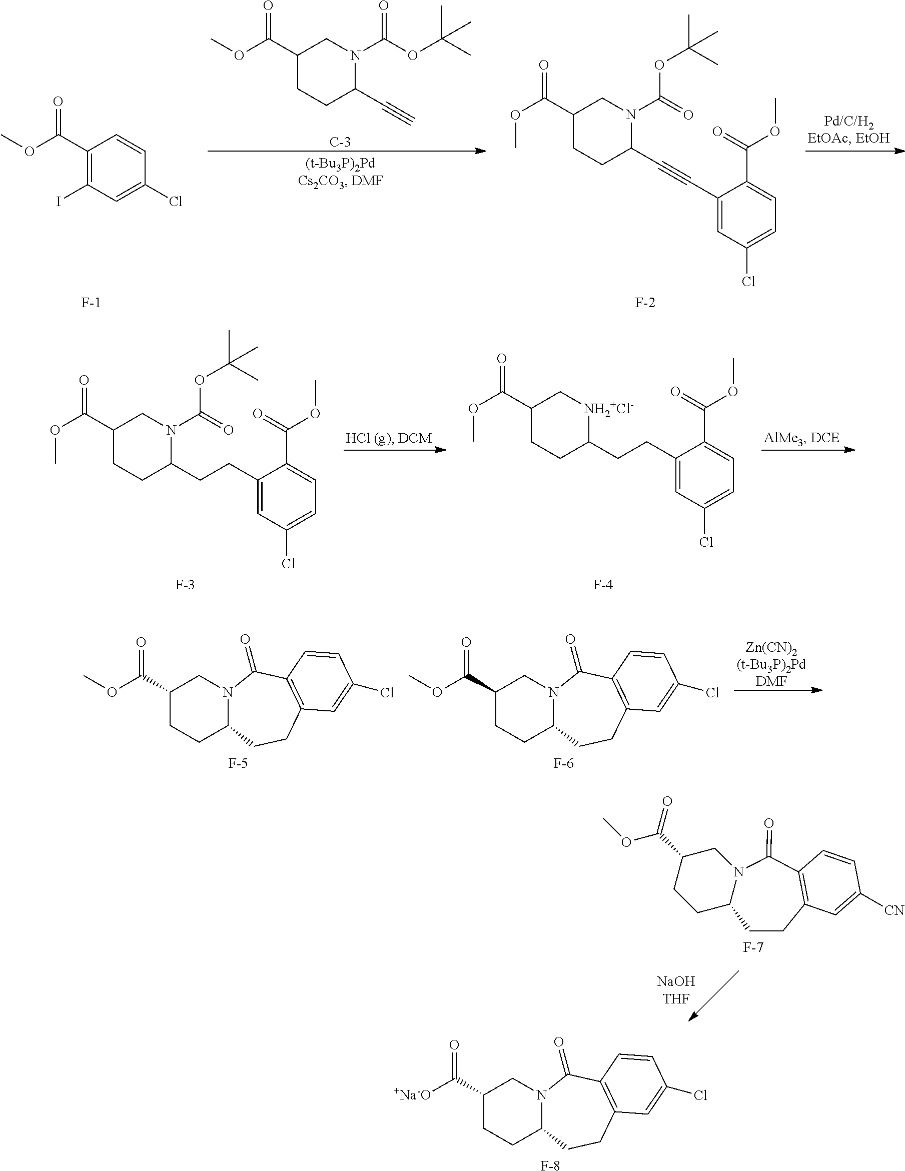

### (±) 3-methyl 1-(2-methyl-2-propanyl)-6-{[5-chloro-2-(methoxycarbonyl)phenyl]ethynyl}-3-piperidinedicarboxylate (F-2)

To a solution of methyl 4-chloro-2-iodobenzoate F-1 (1.0 g, 3.37 mmol) and (±) 1-tert-butyl 3-methyl 6-ethynylpiperidine-1,3-dicarboxylate (C-3, 1.08 g, 4.05 mmol) in DMF (17 mL) was added bis(tri-t-butylphosphine)palladium (0.172 g, 0.337 mmol) and Cs₂CO₃ (2.19 g, 6.75 mmol). The reaction was stirred at 85° C. for 0.5 h. The system was cooled to ambient temperature and partitioned between EtOAc and ice water. The organic phase washed with brine, dried over MgSO₄, filtered and concentrated. The crude residue was purified by silica gel chromatography (0-40% EtOAc in hexanes) to give F-2. MS m/z (M+H) 436.2 found, 436.1 required.

### (±) 3-methyl 1-(2-methyl-2-propanyl)-6-{2-[5-chloro-2-(methoxycarbonyl)phenyl]ethyl}-1,3-piperidinedicarboxylate (F-3)

Intermediate F-2 (0.185 g, 0.424 mmol) was dissolved in 1:1 EtOAc:EtOH (2.0 mL). To this solution was added 10% palladium on charcoal (0.044 g). The reaction vessel was stirred under an atmosphere of hydrogen via a balloon for 5 h. The reaction mixture was filtered through celite and the solution was concentrated. The crude product F-3 was used directly in the next step. MS m/z (M+H) 440.1 found, 440.2 required.

### (±)methyl 6-{2-[5-chloro-2-(methoxycarbonyl)phenyl]ethyl}-3-piperidinium chloride dicarboxylate (F-4)

To a solution of F-3 (0.187 g, 0.425 mmol) in DCM (2.1 mL) was treated with a stream of gaseous HCl for 3 minutes. The system was capped and stirred for 0.5 h and then concentrated in vacuo to afford F-4 as a off-white foam. MS m/z (M+H) 340.1 found, 340.1 required.

### (±) cis-Methyl 9-chloro-6-oxo-1,2,3,4,6,11,12,12a-octahydrobenzo[e]pyrido[1,2-a]azepine-3-carboxylate (F-5)

To a solution of intermediate F-4 (0.160 g, 0.425 mmol) in DCE (4.25 mL) was added trimethylaluminum (2 M in toluene, 0.638 mL). The reaction was stirred at ambient temperature for three days. The reaction was cooled to 0° C. and quenched slowly with 1N HCl (3.0 mL) followed by extraction with DCM. The organic layer was dried over MgSO₄, filtered and concentrated. The crude product was purified by silica gel chromatography (0-50% EtOAc in hexanes) to give F-5 (cis racemate) and F-6 (trans racemate). MS m/z (M+H) 308.0 found, 308.1 required.

### (±) cis-Methyl 9-cyano-6-oxo-1,2,3,4,6,11,12,12a-octahydrobenzo[e]pyrido[1,2-a]azepine-3-carboxylate (F-7)

To a solution of intermediate F-5 (0.020 g, 0.065 mmol) in DMF (0.081 mL) was added Zn(CN)₂ (0.005 g, 0.044 mmol) and bis(tri-t-butylphosphine)palladium (0.003 g, 0.006 mmol). The reaction was heated to 80° C. for 1.5 h. The system was cooled to ambient temperature and then loaded directly onto a silica gel column (0-100% EtOAc in hexanes) to afford F-7 as a clear oil. MS m/z (M+H) 299.1 found, 299.1 required.

### (±) cis sodium-9-cyano-6-oxo-1,2,3,4,6,11,12,12a-octahydrobenzo[e]pyrido[1,2-a]azepine-3-carboxylate (F-8)

To a solution of intermediate F-7 (0.019 g, 0.064 mmol) in THF (0.5 mL) was added aqueous NaOH solution (1N, 0.063 mL) at RT. The system was stirred overnight and then concentrated in vacuo to afford intermediate F-8 as a off-white powder. MS m/z (M+H) 285.0 found, 285.1 required.

### Intermediates G-2 Through G-7

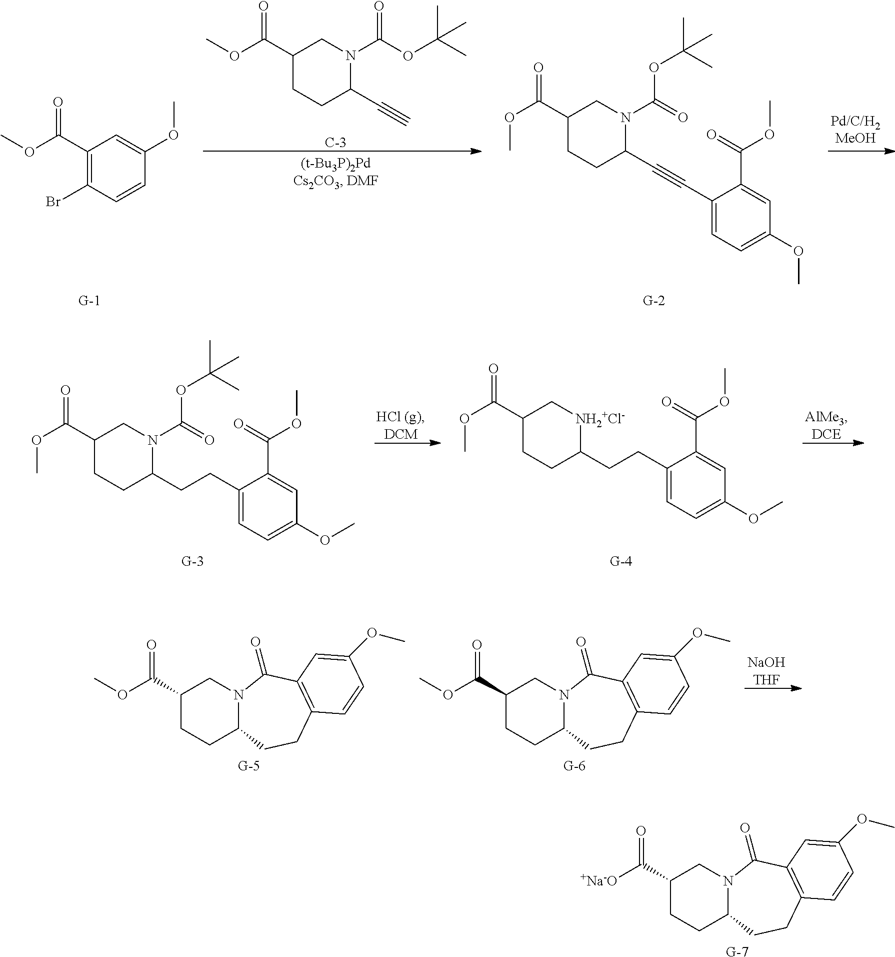

### (±) 3-methyl 1-(2-methyl-2-propanyl)-6-{[4-methoxy-2-(methoxycarbonyl)phenyl]ethynyl}-1,3-piperidinedicarboxlate G-2)

To a solution of methyl 5-methoxy-2-bromobenzoate G-1 (0.80 g, 3.26 mmol) and (±) 1-tert-butyl 3-methyl 6-ethynylpiperidine-1,3-dicarboxylate (C-3, 1.04 g, 3.92 mmol) in DMF (16 mL) was added bis(tri-t-butylphosphine)palladium (0.167 g, 0.326 mmol) and Cs₂CO₃ (2.12 g, 6.53 mmol). The reaction was stirred at 85° C. for 0.5 h. The system was cooled to ambient temperature and partitioned between EtOAc and ice water. The organic phase washed with brine, dried over MgSO₄, filtered and concentrated. The crude residue was purified by silica gel chromatography (0-40% EtOAc in hexanes) to give G-2 as a yellow oil. MS m/z (M+H) 432.1 found, 432.1 required.

### (±) 3-methyl 1-(2-methyl-2-propanyl)6-{2-[4-methoxy-2-(methoxycarbonyl)phenyl]ethyl}-1,3-piperidinedicarboxylate (G-3)

Intermediate G-2 (0.185 g, 0.429 mmol) was dissolved in MeOH (2.0 mL). To this solution was added 10% palladium on charcoal (0.046 g). The reaction vessel was stirred under an atmosphere of hydrogen via a balloon for 6 h. The reaction mixture was filtered through celite and the solution was concentrated. The crude product G-3 was used directly in next step. MS m/z (M+H) 436.0 found, 436.2 required.

### (±)methyl 6-{2-[4-methoxy-2-(methoxycarbonyl)phenyl]ethyl}-3-piperidinium chloride dicarboxylate (G-4)

To a solution of G-3 (0.141 g, 0.379 mmol) in DCM (3.8 mL) was treated with a stream of gaseous HCl for 3 minutes. The system was capped and stirred for 0.5 h and then concentrated in vacuo to afford G-4 as a off-white foam. MS m/z (M+H) 336.0 found, 336.1 required.

### (±) cis-Methyl 10-methoxy-6-oxo-1,2,3,4,6,11,12,12a-octahydrobenzo[e]pyrido[1,2-a]azepine-3-carboxylate (G-5)

To a solution of intermediate G-4 (0.141 g, 0.379 mmol) in DCE (3.80 mL) was added trimethylaluminum (2 M in toluene, 0.569 mL). The reaction was stirred at ambient temperature for 18 h. The reaction was cooled to 0° C. and quenched slowly with 1N HCl (3.0 mL) followed by extraction with DCM. The organic layer was dried over MgSO₄, filtered and concentrated. The crude product was purified by silica gel chromatography (0-50% EtOAc in hexanes) to give G-5 (cis racemate) and G-6 (trans racemate). MS m/z (M+H) 304.0 found, 304.1 required.

### (±) cis sodium-10-methoxy-6-oxo-1,2,3,4,6,11,12,12a-octahydrobenzo[e]pyrido[1,2-a]azepine-3-carboxylate (G-7)

To a solution of intermediate G-5 (0.045 g, 0.148 mmol) in TIE (1.4 mL) was added aqueous NaOH solution (1N, 0.297 mL) at ambient temperature. The system was stirred overnight and then concentrated in vacuo to afford intermediate G-7 as a off-white powder. MS m/z (M+H) 290.1 found, 290.1 required.

### Intermediates H-1 Through H-4

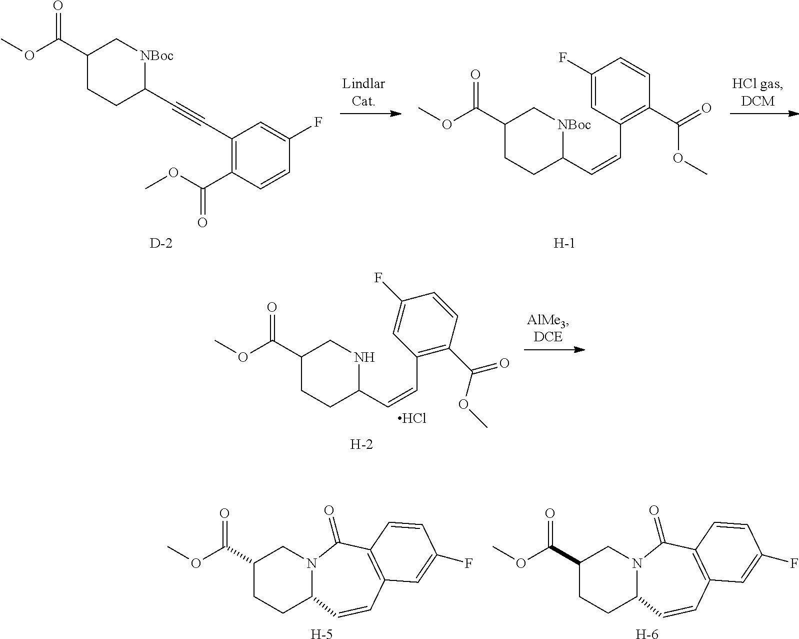

### 1-tert-butyl 3-methyl 6-{(Z)-2-[5-fluoro-2-(methoxycarbonyl)phenyl]ethenyl}piperidine-1,3-dicarboxylate (H-1)

Nitrogen was bubbled through a stirred solution of D-2 (2.5 g, 5.96 mmol) in equal parts of EtOAc (42 mL) and MeOH (42 mL) for several minutes. To this was added Lindlar's catalyst (0.444 g, 4.17 mmol), and allowed to stir under N₂. The reaction flask was purged of N₂ and filled with H₂ several times, and allowed to stir. The reaction was followed by LCMS and NMR every 30 minutes until complete conversion to the desired alkene H-1. The reaction was then filtered through celite and condensed. MS m/z (M+H) 422.1 found, 422.1 required.

### 3-{(Z)-2-[5-fluoro-2-(methylcarbonyl)phenyl]ethenyl]-5-methoxycarbonyl}piperidinium chloride (H-2)

H-1 (2.5 g, 5.96 mmol) was dissolved in DCM and HCl gas was bubbled through for 4 minutes. The reaction was then stirred for 15-30 min before the solvent was removed, residue re-diluted and removed again to give the desired product H-2, MS m/z (M+H) 322.1 found, 322.1 required.

### (±) cis-Methyl 9-fluoro-6-oxo-1,2,3,4,6,11,12,12a-hexahydrobenzo[e]pyrido[1,2-a]azepine-3-carboxylate (H-3)

A solution of intermediate H-2 (2.04 g, 5.91 mmol) in DCE was stirred at room temperature. To this solution was added trimethylaluminium (2 M in toluene, 8.87 mL, 17.7 mmol). The reaction solution was stirred at room temperature for 1 hour, then was heated to 50° C. for 2 hours. The reaction was cooled to room temperature and quenched with drop wise addition of a saturated solution of Rochelle's salts. The phases were separated and the aqueous phase was extracted with EtOAc three times. The organic layer was dried over Na₂SO₄ and concentrated. The crude product was purified by silica gel chromatography (120 g column, 0-70% EtOAc in hexane) to give H-3 (cis racemate) and F-5 (trans racemate) MS m/z (M+H) 290.1 found, 290.1 required.

### Intermediates I-1 Through I-3

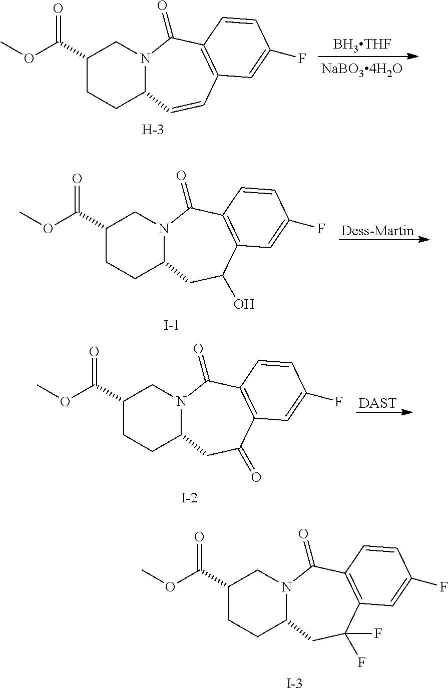

### (±) cis-Methyl-9-fluoro-11-hydroxy-6-oxo-1,2,3,4,6,11,12,12a-octahydropyrido[1,2-b][2]benzazepine-3-carboxylate (I-1)

A solution of H-3 (0.4 g, 1.38 mmol) in THF was stirred under N₂. To this was added 1M borane THF complex (2.77 ml, 2.77 mmol) drop wise, and stirred overnight. To this was added NaBO₃-4H₂O (0.638 g, 4.15 mmol), and stirred for 30 minutes. The reaction was then diluted with diethyl ether, and washed with a very small amount of H₂O. The aqueous layer was then washed with diethyl ether three times. The combined organic layers were then washed with brine, dried over Na₂SO₄, filtered and condensed. The crude material was purified on silica gel column (24 g, 0-100% EtOAc in hexane) to give I-1 as an oil, MS m/z (M+H) 308.0 found, 308.1 required.

### (±) cis-Methyl-9-fluoro-6,11-dioxo-1,2,3,4,6,11,12,12a-octahydropyrido[1,2-b][2]benzazepine-3-carboxylate (I-2)

A solution of alcohol I-1 (70 mg, 0.228 mmol) in DCM (2.2 mL) was treated with NaHCO₃ (19.13 mg, 0.228 mmol) and stirred for 10 minutes, followed by addition of Dess-Martin periodinane (290 mg, 0.683 mmol). The reaction was followed by LCMS. When all starting alcohol was consumed the reaction was treated with saturated aqueous Na₂S₂O₃, and saturated aqueous NaHCO₃, and stirred for 15 minutes. The phases were separated, and the aqueous layer was extracted with EtOAc three times. The combined organic layer was then washed with saturated aqueous NaHCO₃ and water. The organic layer was dried over Na₂SO₄ filtered and condensed. Purification with silica gel column (12 g column 0-35% EtOAc in hexane) gave 1-2, MS m/z (M+H) 306.0 found, 306.1 required.

### (±) cis-Methyl-9,11,11-trifluoro-6-oxo-1,2,3,4,6,11,12,12a-octahydropyrido[1,2-b][2]benzazepine-3-carboxylate (I-3)

A stirred solution of I-2 (72 mg, 0.236 mmol) in DCE (2.3 ml) under N₂ was treated with DAST (125 μL, 0.943 mmol) and heated with stirring to 45° C. The reaction was run overnight, then cooled and worked up with slow addition of saturated NaHCO₃. The aqueous phase was extracted with DCM, then EtOAc and dried over MgSO₄, filtered and concentrated. The product was purified using reverse phase HPLC (0-70% 0.1% TFA acetonitrile in 0.1% TFA H₂O). The correct fractions were diluted with EtOAc, and washed with saturated aqueous NaHCO₃, and the organic phase was dried over MgSO₄, and condensed to give I-3. MS m/z (M+H) 328.1 found, 328.1 required.

### Example 1

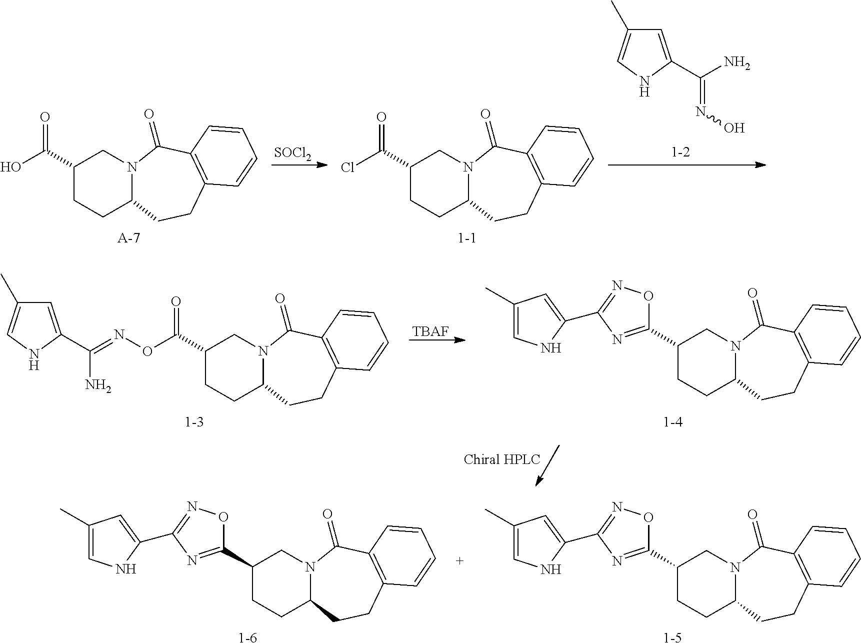

### (±) cis-6-oxo-1,2,3,4,6,11,12,12a-Octahydrobenzo[e]pyrido[1,2-a]azepine-3-carbonyl chloride (1-1)

A solution of A-7 (310 mg, 1.2 mmol) in CH₂Cl₂ (15 mL) was cooled to 0° C. To this solution was added thionyl chloride (0.45 mL, 6.2 mmol). The reaction was allowed to warm to RT and stirred for 1 h. The reaction was concentrated to give crude 1-1 which was used directly in next step.

(±) N′-((cis-6-oxo-1,2,3,4,6,11,12,12a-Octahydrobenzo[e]pyrido[1,2-a]azepine-3-carbonyl)oxy)-4-methyl-1H-pyrrole-2-carboximidamide (1-3)

A suspension of N′-hydroxy-4-methyl-1H-pyrrole-2-carboximidamide 1-2 (232 mg, 1.7 mmol, prepared as described in WO 2006/123257) in CH₂Cl₂ (3 mL) and THF (0.6 mL) was cooled to 0° C. To this was added TEA (0.24 mL, 1.7 mmol) and a solution of 1-1 (332 mg, 1.2 mmol) in THF (1.0 mL). The clear solution was allowed to stir for 30 min. The reaction was partitioned between CH₂Cl₂ and water. The organic layer was washed with brine and concentrated to give crude 1-3. m/z (M+H) 381.2 found, 381.4 required.

### (±) cis-3-(3-(4-Methyl-1H-pyrrol-2-yl)-1,2,4-oxadiazol-5-yl)-1,3,4,11,12,12a-hexahydrobenzo[e]pyrido[1,2-a]azepin-6(2H)-one (1-4)

1M TBAF solution in THF (2.4 mL) was added to a solution of 1-3 (450 mg, 1.2 mmol) in THF at 0° C. The reaction solution was stirred for one hour and then partitioned between EtOAc and water. The organic layer was washed with brine and concentrated. The crude product was purified by silica gel chromatography (50% EtOAc in heptane) to give cis racemate 1-4. MS m/z (M+H) 363.1816 found, 363.1816 required.

### cis-(3S,12aS)-3-(3-(4-Methyl-1H-pyrrol-2-yl)-1,2,4-oxadiazol-5-yl)-1,3,4,11,12,12a-hexahydrobenzo[e]pyrido[1,2-a]azepin-6(2H)-one (1-5) and cis-(3R,12aR)3-(3-(4-Methyl-1H-pyrrol-2-yl)-1,2,4-oxadiazol-5-yl)-1,3,4,11,12,12a-hexahydrobenzo[e]pyrido[1,2-a]azepin-6(2H)-one (1-6)

The single enantiomers 1-5 and 1-6 were obtained from chiral HPLC separation of racemic 1-4. 1-5: MS m/z (M+H) 363.1818 found, 363.1816 required.

### Example 2

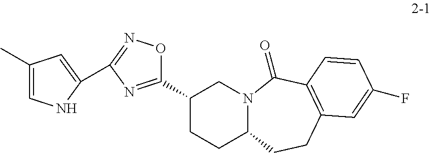 [SMILES: Cc1c[nH]c(-c2noc([C@H]3CC[C@H]4CCc5cc(F)ccc5C(=O)N4C3)n2)c1]

### (3S,12aS)-9-Fluoro-3-[3-(4-methyl-1H-pyrrol-2-yl)-1,2,4-oxadiazol-5-yl]-1,3,4,11,12,12a-hexahydropyrido[1,2-b][2]benzazepin-6(2H)-one (2-1)

Compound 2-1 was prepared essentially as described in Example 1 using D-7 and 1-2. NMR (CDCl₃) δ=8.78 (br s, 1H), 7.65 (dd, 1H, J=5.9 and 8.3 Hz), 7.02 (m, 1H), 6.86 (d, 1H, J=9.6 Hz), 6.75 m, 2H), 5.06 d, 1H, J=9.6 Hz), 3.72 (br m, 1H), 3.18-3.32 (m, 2H), 2.82 (m, 1H), 2.71 (m, 1H), 2.39 (m, 1H), 2.15 (s, 3H), 1.96 (m, 2H) and 1.78 (m, 2H) ppm. MS m/z (M+H) 363.1816 found, 363.1816 required.

### Example 3

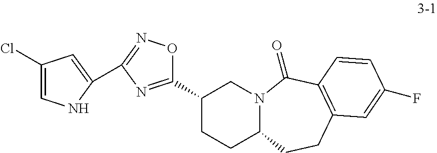 [SMILES: O=C1c2ccc(F)cc2CC[C@@H]2CC[C@H](c3nc(-c4cc(Cl)c[nH]4)no3)CN12]

### (3S,12aS)-9-Fluoro-3-[3-(4-chloro1H-pyrrol-2-yl)-1,2,4-oxadiazol-5-yl]-1,3,4,11,12,12a-hexahydropyrido[1,2-b][2]benzazepin-6(2H)-one (3-1)

Compound 3-1 was prepared essentially as described in Example 1 using A-7 and 4-chloro-N′-hydroxy-1H-pyrrole-2-carboximidamide. NMR (CDCl₃) δ=8.94 (br s, 1H), 7.65 (dd, 1H, J=5.8 and 8.5 Hz), 7.02 (m, 1H), 6.92 (m, 1H), 6.75 (m, 2H), 5.06 (m, 1H), 3.71 (m, 1H), 3.16-3.32 (m, 2H), 2.82 (m, 1H), 2.70 (m, 1H), 2.39 (m, 1H), 2.16 (m, 1H), 1.95 (m, 2H) and 1.78 (m, 2H) ppm. MS m/z (M+H) 363.1816 found, 363.1816 required.

### Example 4

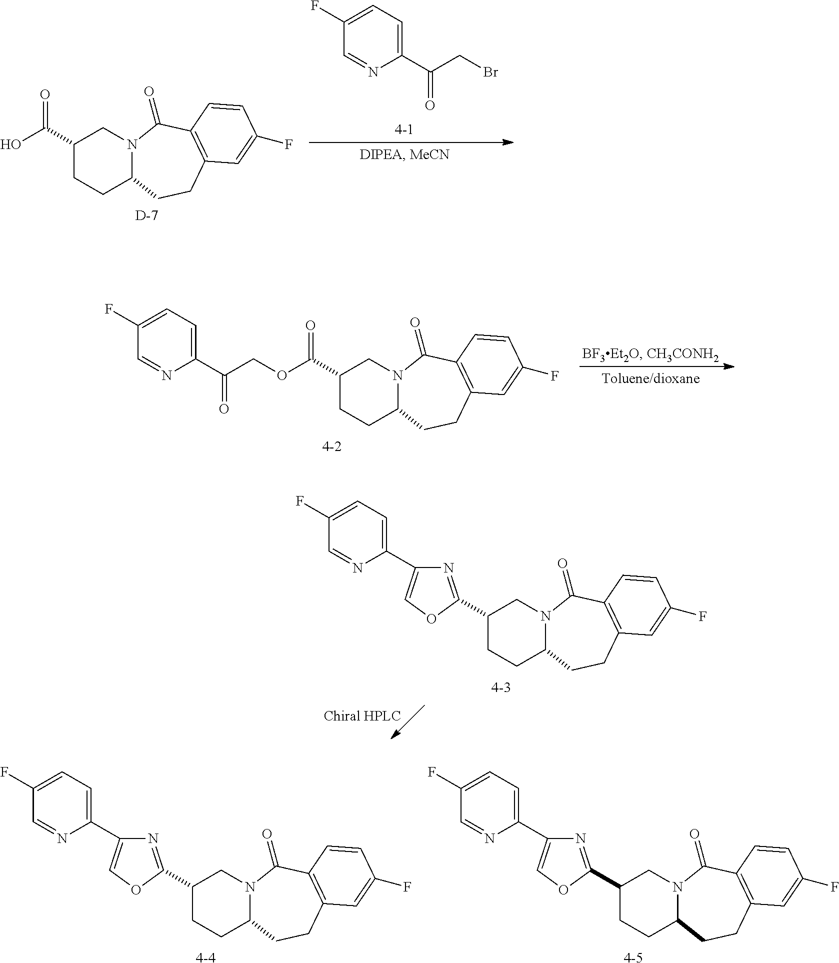

### (±) cis-2-(5-fluoropyridin-2-yl)-2-oxoethyl 9-fluoro-6-oxo-1,2,3,4,6,11,12,12a-octahydrobenzo[e]pyrido[1,2-a]azepine-3-carboxylate (4-2)

A solution of D-7 (238 mg, 0.86 mmol), 2-bromo-1-(5-fluoropyridin-2-yl)ethanone 4-1 and DIPEA (0.25 mL, 1.44 mmol) in CH₃CN (4 mL) was stirred at RT overnight. The reaction was partitioned between EtOAc and water. The organic layer was washed with brine and concentrated to give crude 4-2 which was used directly in next step. m/z (M+H) 415.1 found, 415.1 required.

### (±) cis-9-fluoro-3-(4-(5-fluoropyridin-2-yl)oxazol-2-yl)-1,3,4,11,12,12a-hexahydrobenzo[e]pyrido[1,2-a]azepin-6(2H)-one (4-3)

To a suspension of (±) cis-2-(5-fluoropyridin-2-yl)-2-oxoethyl 9-fluoro-6-oxo-1,2,3,4,6,11,12,12a-octahydrobenzo[e]pyrido[1,2-a]azepine-3-carboxylate 4-2 (356 mg, 0.86 mmol) and acetamide (1.0 g, 17 mmol) in toluene (6 mL) and dioxane (3 mL) was added BF₃-Et₂O (0.22 mL, 1.7 mmol). The mixture was heated under microwave at 155° C. for 1 hour. The reaction was partitioned between EtOAc and water. The organic layer was washed with brine and concentrated. The crude product was purified by silica gel column to give 4-3 (110 mg) as off white solid. m/z (M+H) 396.1 found, 396.1 required.

### cis-(3S,12aS)-9-fluoro-3-(4-(5-fluoropyridin-2-yl)oxazol-2-yl)-1,3,4,11,12,12a-hexahydrobenzo[e]pyrido[1,2-a]azepin-6(2H)-one (4-4) and cis-(3R,12aR)-9-fluoro-3-(4-(5-fluoropyridin-2-yl)oxazol-2-yl)-1,3,4,11,12,12a-hexahydrobenzo[e]pyrido[1,2-a]azepin-6(2H)-one (4-5)

The single enantiomers 4-4 and 4-5 were obtained from chiral HPLC separation of racemic 4-3. Preparative HPLC was performed on OD-H column (3 cm×25 cm, eluted with 40% Hexane and 60% EtOH (containing 0.1% TFA) at 42 mL/min flow rate. 4-4 (peak one): MS m/z (M+H) 396.1518 found, 396.1518 required. 4-5 (peak two): MS m/z (M+H) 396.1518 found, 396.1518 required.

### Example 5

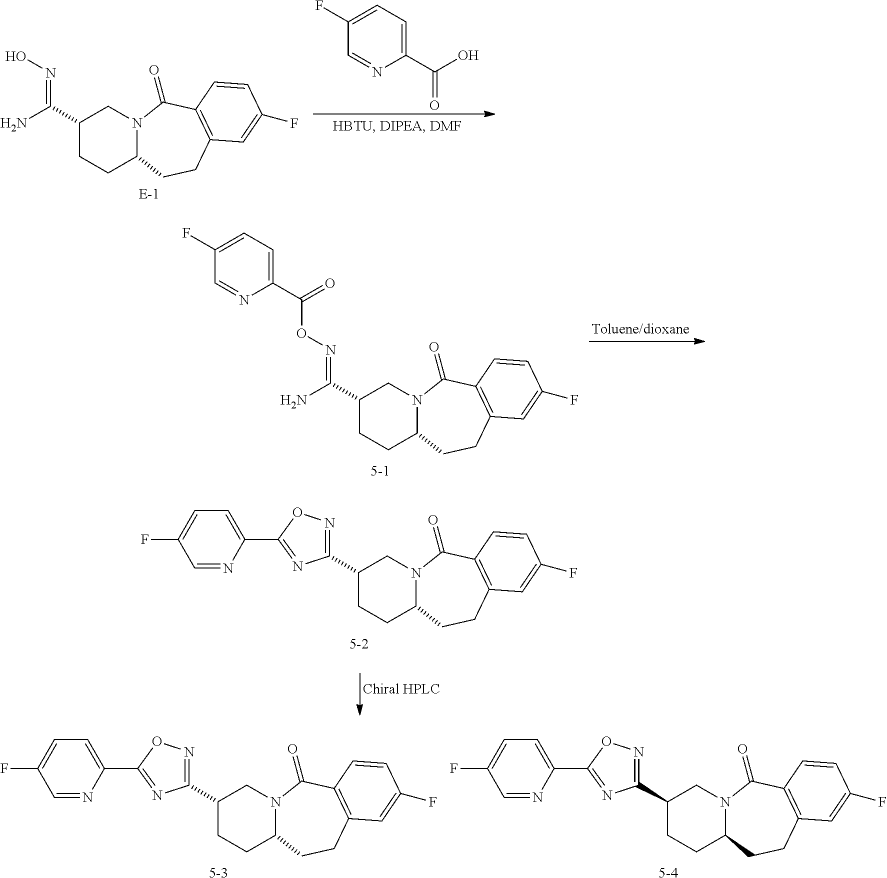

### (±) cis-9-fluoro-N′-((5-fluoropicolinoyl)oxy)-6-oxo-1,2,3,4,6,11,12,12a-octahydrobenzo[e]pyrido[1,2-a]azepine-3-carboximidamide 5-1

To a solution of 5-fluoropicolinic acid (2.87 g, 20.3 mmol) in DMF (75 mL) was added HATU (7.56 g, 19.9 mmol). After stirred at RT for 10 min, the solution was cooled to 0° C. To this solution was added DIPEA (4.91 mL, 28.2 mmol) and E-1 (5.48 g, 18.8 mmol). The reaction was stirred for 1 hour and added EtOAc and water. The solid was filtered to give crude product as off white solid (5.8 g). m/z (M+H) 415.3 found, 415.2 required.

### (±) cis-9-fluoro-3-(5-(5-fluoropyridin-2-yl)-1,2,4-oxadiazol-3-yl)-1,3,4,11,12,12a-hexahydrobenz(±) cis-o[e]pyrido[1,2-a]azepin-6(2H)-one (5-2)

A suspension of (±) cis-9-fluoro-N′-((5-fluoropicolinoyl)oxy)-6-oxo-1,2,3,4,6,11,12,12a-octahydrobenzo[e]pyrido[1,2-]azepine-3-carboximidamide 5-1 (5.8 g, 14 mmol) in toluene (100 mL) and 1,4-dioxane (50 mL) was heated at 110° C. overnight. Solvent was removed to give crude 5-2. m/z (M+H) 397.3 found, 397.1 required.

### cis-(3S,12aS)-9-fluoro-3-(5-(5-fluoropyridin-2-yl)-1,2,4-oxadiazol-3-yl)-1,34,11,12,12a-hexahydrobenzo[e]pyrido[1,2-a]azepin-6(2H)-one (5-3) and cis-(3R,12aR)-9-fluoro-3-(5-(5-fluoropyridin-2-yl)-1,2,4-oxadiazol-3-yl)-1,3,4,11,12,12a-hexahydrobenzo[e]pyrido[1,2-a]azepin-6(2H)-one (5-4)

The single enantiomers 5-3 and 5-4 were obtained from chiral HPLC separation of racemic 5-2. Preparative HPLC was performed on OD-H column (3 cm×25 cm, eluted with 60% CO₂ and 40% MeOH at 80 mL/min flow rate. 5-3 (peak one): MS m/z (M+H) 397.1468 found, 397.1471 required. 5-4 (peak two): MS m/z (M+H) 397.1470 found, 397.1471 required.

### Example 6

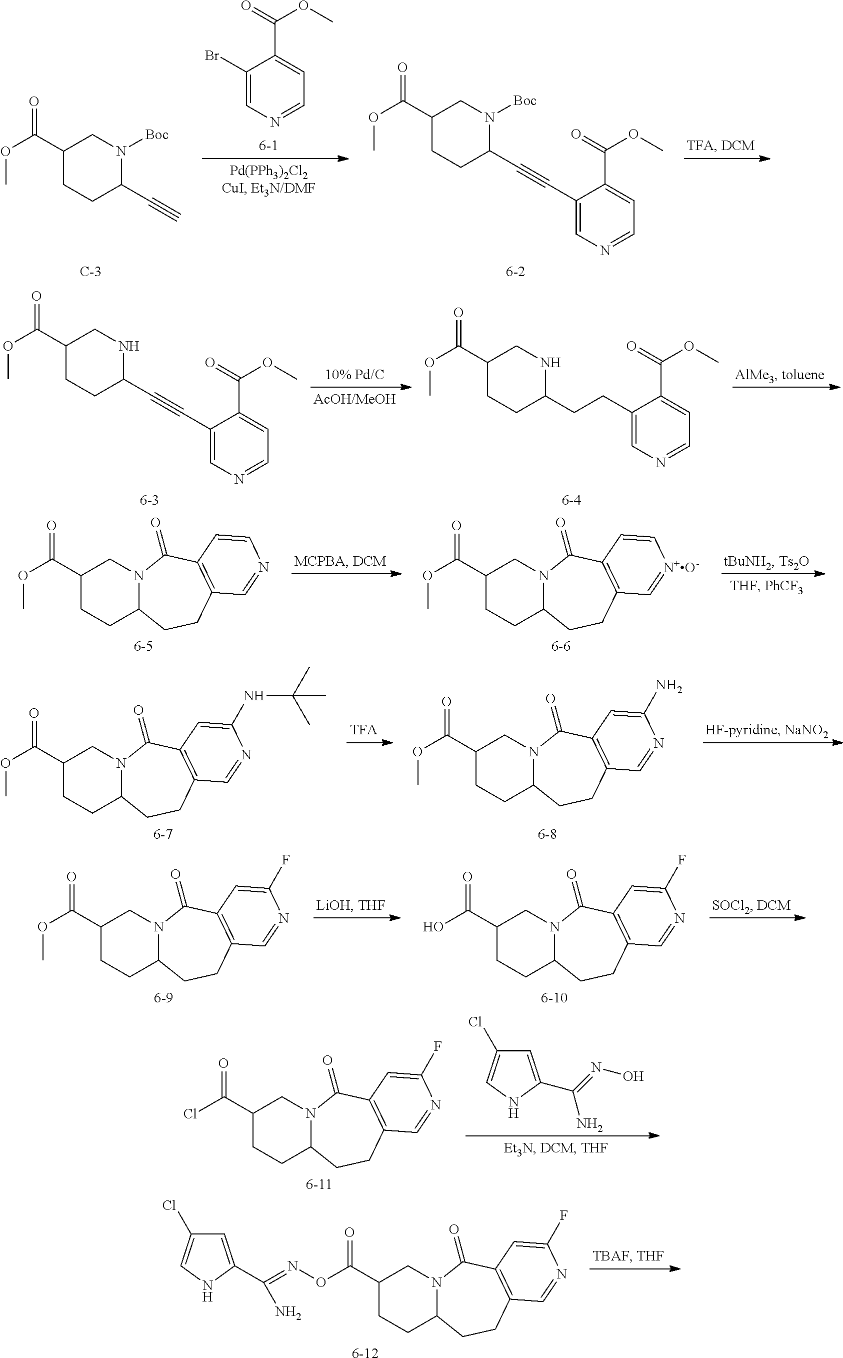

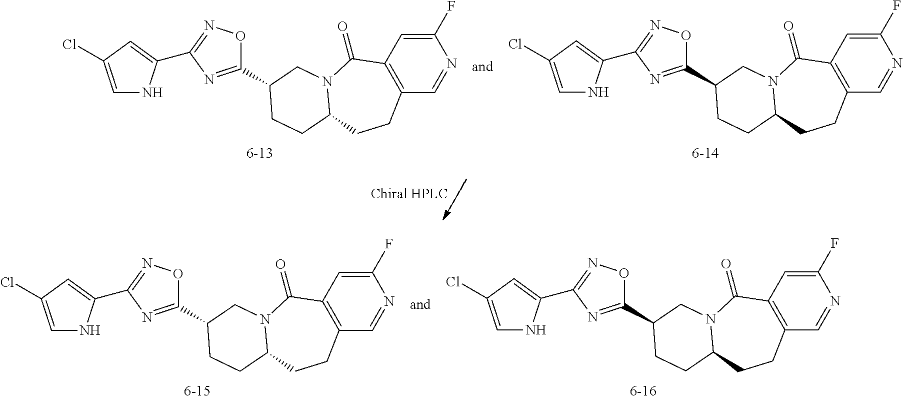 [SMILES: O=C1c2cc(F)ncc2CC[C@@H]2CC[C@H](c3nc(-c4cc(Cl)c[nH]4)no3)CN12 ; O=C1c2cc(F)ncc2CC[C@H]2CC[C@@H](c3nc(-c4cc(Cl)c[nH]4)no3)CN12 ; O=C1c2cc(F)ncc2CC[C@@H]2CC[C@H](c3nc(-c4cc(Cl)c[nH]4)no3)CN12 ; O=C1c2cc(F)ncc2CC[C@H]2CC[C@@H](c3nc(-c4cc(Cl)c[nH]4)no3)CN12]

### 1-tert-butyl 3-methyl 6-((4-(methoxycarbonyl)pyridin-3-yl)ethynyl)piperidine-1,3-dicarboxylate (6-2)

To a solution of methyl 3-bromoisonicotinate 6-1 (1.25 g, 5.8 mmol) in DMF (12 mL) was added Et₃N (8.0 mL), Pd(PPh₃)₂Cl₂ (285 mg, 0.4 mmol) and CuI (77 mg, 0.4 mmol). After stirred for 10 min at RT, C-3 (1.55 g, 5.8 mmol) was added. The yellow solution was stirred at RT overnight. The reaction mixture was partitioned between EtOAc and water. The organic phase was washed with water and brine successfully and concentrated. The crude product was purified by silica gel chromatography to give 6-2 (1.8 g) as mixture of cis and trans isomers. m/z (M+H) 403.1 found, 403.2 required.

### 1-tert-butyl 3-methyl 6-(2-(4-(methoxycarbonyl)pyridin-3-yl)ethyl)piperidine-1,3-dicarboxylate (6-3)

To a solution of 1-tert-butyl 3-methyl 6-((4-(methoxycarbonyl)pyridin-3-yl)ethynyl)piperidine-1,3-dicarboxylate 6-2 (1.8 g, 4.5 mmol) in DCM (33 mL) was added TFA (15 mL, 195 mmol). The solution was stirred at RT for 40 min. The reaction solution was concentrated and the crude product 6-3 was used in next step directly. MS m/z (M+H) 303.0 found, 303.1 required.

### methyl 3-(2-(5-(methoxycarbonyl)piperidin-2-yl)ethyl)isonicotinate (6-4)

The mixture of 1-tert-butyl 3-methyl 6-(2-(4-(methoxycarbonyl)pyridin-3-yl)ethyl)piperidine-1,3-dicarboxylate 6-3 (4.5 mmol), 10% Pd/C (500 mg) and 1N HCl (5 mL) in MeOH (60 mL) was hydrogenated on a Parr shaker at 45 psi for 48 h. The catalyst was removed by filtration. The filtration was concentrated and partitioned between DCM and sat. aq NaHCO₃. The organic phase was dried and concentrated to give crude 6-4 (1.4 g) as mixture of cis and trans isomers. MS m/z (M+H) 307.0 found, 307.2 required.

### methyl 5-oxo-5,7,8,9,10,10a,11,12-octahydrodipyrido[1,2-a:3′,4′-e]azepine-8-carboxylate (6-5)

To a solution of methyl 3-(2-(5-(methoxycarbonyl)piperidin-2-yl)ethyl)isonicotinate 6-4 (1.55 g, 5.1 mmol) in DCM (50 mL) cooled over a dry ice/acetone bath was added trimethylalluminum (2M in toluene, 3.8 mL, 7.6 mmol). After addition was complete, the cooling bath was removed and the reaction was stirred at 25° C. for 4 hours. After this time, the reaction mixture was quenched with crushed ice and filtered through celite. The aqueous phase was extracted with DCM. The combined extracts were dried over MgSO₄, filtered and the solvent removed in vacuo. This residue was chromatographed on silica gel (5% MeOH/DCM) to give 6-5 (0.97 g, 70%) as mixture of cis and trans isomers. MS m/z (M+H) 275.0 found, 275.1 required.

### 8-(methoxycarbonyl)-5-oxo-5,7,8,9,10,10a,11,12-octahydrodipyrido[1,2-a:3′,4′-e]azepine 2-oxide (6-6)

To a solution of methyl 5-oxo-5,7,8,9,10,10a,11,12-octahydrodipyrido[1,2-a:3′,4′-e]azepine-8-carboxylate 6-5 (0.97 g, 3.5 mmol) in DCM (30 mL) cooled over an ice/water bath was added MCPBA (0.96 g, 3.9 mmol). After addition was complete, the cooling bath was removed and the reaction was stirred at RT for 2 hours. The reaction mixture was concentrated and the residue was chromatographed on silica gel (5% MeOH/DCM) to give 6-6 (0.98 g, 95%) as mixture of cis and trans isomers.

### methyl 3-(tert-butylamino)-5-oxo-5,7,8,9,10,10a,11,12-octahydrodipyrido[1,2-a:3′,4′-e]azepine-8-carboxylate (6-7)

To a solution of 8-(methoxycarbonyl)-5-oxo-5,7,8,9,10,10a,11,12-octahydrodipyrido[1,2-a:3′,4′-e]azepine 2-oxide 6-6 (0.86 g, 3.0 mmol) and tert-butylamine (2.0 mL, 19.0 mmol) in (trifluoromethyl)benzene (14 mL) and THF (6 mL) was added 4-methylbenzenesulfonic anhydride (3.0 g, 9.2 mmol) in several portions. After addition was complete, the solution was added hexane (10 mL). The precipitation was filtered and rinsed with a mixture of hexane/THF (1:1, ˜15 mL total). The solution was concentrated and crude 6-7 was used in next step directly. MS m/z (M+H) 346.1 found, 346.2 required.

### methyl 3-amino-5-oxo-5,7,8,9,10,10a,11,12-octahydrodipyrido[1,2-a:3′,4′-e]azepine-8-carboxylate (6-8)

The solution of methyl 3-(tert-butylamino)-5-oxo-5,7,8,9,10,10a,11,12-octahydrodipyrido[1,2-a:3′,4′-e]azepine-8-carboxylate 6-7 (3.0 mmol) in TFA (15 mL) was heated at 70° C. for 4 hours. The solution was concentrated and partitioned between EtOAc and 5% aq NaHCO₃. The aqueous phase was extracted with EtOAc. The combined extracts were dried over MgSO₄, filtered and concentrated. This residue was chromatographed on silica gel (5% MeOH/DCM) to give 6-8 (0.69 g, 81% over two steps). MS m/z (M+H) 290.0 found, 290.1 required.

### methyl 3-fluoro-5-oxo-5,7,8,9,10,10a,11,12-octahydrodipyrido[1,2-a:3′,4′-e]azepine-8-carboxylate (6-9)

To a solution of methyl 3-amino-5-oxo-5,7,8,9,10,10a,11,12-octahydrodipyrido[1,2-a:3′,4′-e]azepine-8-carboxylate 6-8 (420 mg, 1.45 mmol) in 70% hydrogen fluoride-pyridine (8 mL) at 0° C. was added sodium nitrite (1 Mg, 14.5 mmol) in three portions. After stirred at 0° C. for 10 min, the reaction solution was poured into cold NH₄OH/water (1:1, 30 mL). The mixture was extracted with EtOAc twice. The combined extracts were washed with brine and dried over MgSO₄. The solution was concentrated and this residue was chromatographed on silica gel (50% EtOAc/Hexane) to give 6-9 (280 mg, 66%) as mixture of cis and trans isomers. MS m/z (M+H) 293.0 found, 293.1 required.

### 3-fluoro-5-oxo-5,7,8,9,10,10a,11,12-octahydrodipyrido[1,2-a:3′,4′-e]azepine-8-carboxylic acid (6-10)

To a solution of methyl 3-fluoro-5-oxo-5,7,8,9,10,10a,11,12-octahydrodipyrido[1,2-a:3′,4′-e]azepine-8-carboxylate 6-9 (280 mg, 0.96 mmol) in THF (8.0 mL) was added 1.0N LiOH (2.0 mL, 2.0 mmol) and stirred at 25° C. for one hour. The solution was treated with 1N HCl (2.0 mL, 2.0 mmol) and water (4 mL), and then extracted with DCM (2×10 mL). The combined extracts were dried over MgSO₄, filtered and the solvent removed in vacuo to give 6-10 (267 mg, 100%). MS m/z (M+H) 278.9 found, 279.1 required.

### 3-fluoro-5-oxo-5,7,8,9,10,10a,11,12-octahydrodipyrido[1,2-a:3′,4′-e]azepine-8-carbonyl chloride (6-11)

To a solution of 3-fluoro-5-oxo-5,7,8,9,10,10a,11,12-octahydrodipyrido[1,2-a:3′,4′-e]azepine-8-carboxylic acid 6-10 (210 mg, 0.76 mmol) in DCM (6 mL) was added thionyl chloride (0.27 mL, 3.77 mmol). The reaction was stirred for 1 hour at RT. To the solution was added toluene (2.0 mL). The mixture was concentrated to give crude 6-11 which was used directly in next step.

### (Z)-4-chloro-N′-(((8S,10aS)-3-fluoro-5-oxo-5,7,8,9,10,10a,11,12-octahydrodipyrido[1,2-a:3′,4′-e]azepine-8-carbonyl)oxy)-1H-pyrrole-2-carboximidamide (6-12)

A solution of N′-hydroxy-4-chloro-1H-pyrrole-2-carboximidamide (32.5 mg, 0.20 mmol, prepared as described in WO 2006/123257) in THF (0.4 mL) was cooled to 0° C. To this was added TEA (0.10 mL, 0.74 mmol) and a solution of 3-fluoro-5-oxo-5,7,8,9,10,10a,11,12-octahydrodipyrido[1,2-a:3′,4′-e]azepine-8-carbonyl chloride 6-11 (332 mg, 1.2 mmol) in DCM (0.7 mL). The clear solution was allowed to stir for 30 min. The reaction was partitioned between DCM and water. The organic layer was washed with brine and concentrated to give crude 6-12. m/z (M+H) 420.1 found, 420.1 required.

### (±) cis-8-(3-(4-chloro-1H-pyrrol-2-yl)-1,2,4-oxadiazol-5-yl)-3-fluoro-8,9,10,10a,11,12-hexahydrodipyrido[1,2-a:3′,4′-e]azepin-5(7H)-one (6-13) and (±) trans-8-(3-(4-chloro-1H-pyrrol-2-yl)-1,2,4-oxadiazol-5-yl)-3-fluoro-8,9,10,10a,11,12-hexahydrodipyrido[1,2-a:3′,4′-e]azepin-5(7H)-one (6-14)

1M TBAF solution in THF (0.13 mL) was added to a solution of (Z)-4-chloro-N′-hydroxy-1H-pyrrole-2-carboximidamide (Z)-4-chloro-N′-(((8S,10aS)-3-fluoro-5-oxo-5,7,8,9,10,10a,11,12-octahydrodipyrido[1,2-a:3′,4′-e]azepine-8-carbonyl)oxy)-1H-pyrrole-2-carboximidamide 6-12 (56 mg, 0.13 mmol) in THF (2.0 mL) at 0° C. The reaction solution was stirred for one hour and then partitioned between EtOAc and water. The organic layer was washed with brine and concentrated. The crude product was purified by silica gel chromatography (50% EtOAc in heptane) to give cis racemate 6-13 and trans racemate 6-14. MS m/z (M+H) 402.0 found, 402.1 required.

### cis-(8S,10aS)-8-(3-(4-chloro-1H-pyrrol-2-yl)-1,2,4-oxadiazol-5-yl)-3-fluoro-8,9,10,10a,11,12-hexahydrodipyrido[1,2-a:3′,4′-e]azepin-5(7H)-one (6-15) and cis-(8R,10aR)-8-(3-(4-chloro-1H-pyrrol-2-yl)-1,2,4-oxadiazol-5-yl)-3-fluoro-8,9,10,10a,11,12-hexahydrodipyrido[1,2-a:3′,4′-e]azepin-5(7H)-one (6-16)

The single enantiomers 6-15 and 6-16 were obtained from chiral HPLC separation of racemic 6-13. Preparative HPLC was performed on AS-H column (5 cm×50 cm, eluted with 100% MeOH (containing 0.1% TFA) at 120 mL/min flow rate. 6-15 (peak one): MS m/z (M+H) 402.1125 found, 402.1128 required. 6-16 (peak two): MS m/z (M+H) 402.1126 found, 402.1128 required.

### Example 7

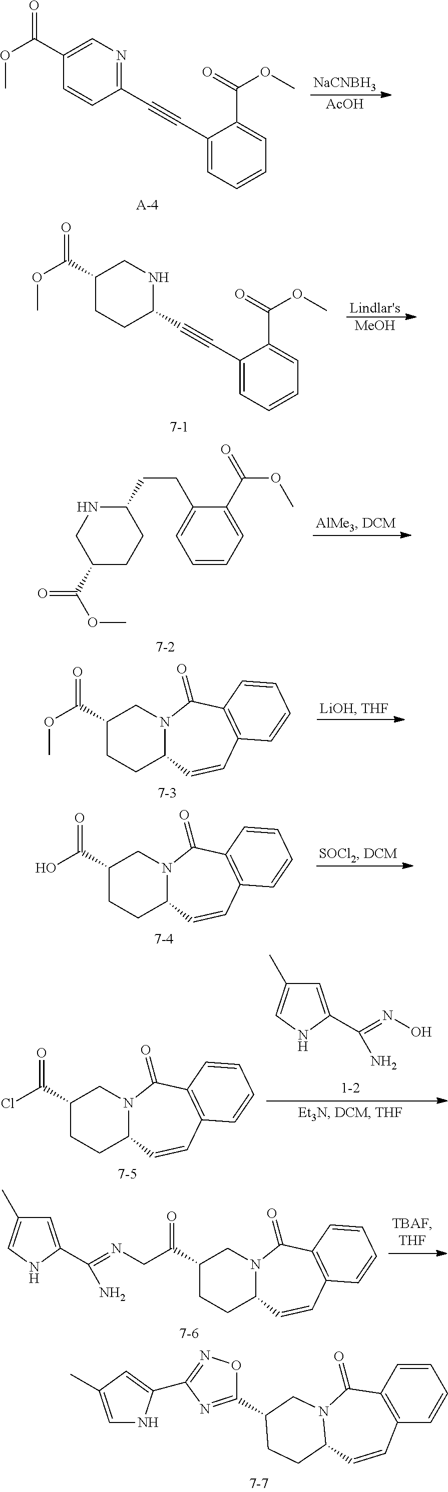

### (±) cis-Methyl 6-((2-(methoxycarbonyl)phenyl)ethynyl)piperidine-3-carboxylate (7-1)

To a solution of methyl 6-{[2-methoxycarbonyl)phenyl]ethynyl}pyridine-3-carboxylate A-4 (270 mg, 0.91 mmol) in AcOH (5.0 mL) was added NaCNBH₃ (345 mg, 5.5 mmol) in two portions. The mixture was stirred at RT for 2 hours. The reaction was diluted with 10 mL water and adjusted pH=8 with NaHCO₃. The mixture was extracted with EtOAc twice. The combined organic phase was washed with brine and concentrated. The crude product containing cis and trans isomers was separated by chromatography on silica gel to give 7-1 (106 mg). MS m/z (M+H) 302.1 found, 302.1 required.

### (±) cis-(E)-Methyl 6-(2-(methoxycarbonyl)styryl)piperidine-3-carboxylate (7-2)

To a solution of (±) cis-methyl 6-((2-(methoxycarbonyl)phenyl)ethynyl)piperidine-3-carboxylase (7-1) (156 mg, 0.52 mmol) in MeOH (5.0 mL) was added Lindlar's catalyst (100 mg). The mixture was allowed to stir overnight under 1 atm hydrogen atmosphere. The solid was removed by filtration and the filtrate was concentrated to give crude 7-2 (120 mg, 76%). m/z (M+H) 304.0 found, 304.1 required.

### (±) cis-Methyl 6-oxo-1,2,3,4,6,12a-hexahydrobenzo[e]pyrido[1,2-a]azepine-3-carboxylate (7-3)

To a solution of (±) cis-(E)-methyl 6-(2-(methoxycarbonyl)styryl)piperidine-3-carboxylate 7-2 (120 mg, 0.40 mmol) in DCM (5.0 mL) cooled over a dry ice/acetone bath was added trimethylaluminum (2M in toluene, 0.30 mL, 0.60 mmol). After addition was complete, the cooling bath was removed and the reaction was stirred at 25° C. for 1 hours. After this time, the reaction mixture was quenched with crushed ice and 1N HCl (3.0 mL). The aqueous was extracted with DCM again. The combined extracts were dried over MgSO₄, filtered and the solvent removed in vacuo. This residue was chromatographed on silica gel (25% EtOAc/Hexane) to give 7-3 (78 mg, 73%) as white solid. MS m/z (M+H) 272.0 found, 272.1 required.

### (±) cis-6-oxo-1,2,3,4,6,12a-hexahydropyrido[1,2-b][2]benzazepine-3-carboxylic acid (7-4)

To a solution of (±) cis-methyl 6-oxo-1,2,3,4,6,12a-hexahydrobenzo[e]pyrido[1,2-a]azepine-3-carboxylate 7-3 (78 mg, 0.29 mmol) in MeOH (2.0 mL) was added 1.0N LiOH (0.58 mL, 0.58 mmol). The solution was stirred at 25° C. for one hour and treated with 1.0N HCl (0.70 mL, 0.70 mmol) and water (2 mL). The mixture was extracted with DCM twice. The combined extracts were dried over MgSO₄, filtered and the solvent removed in vacuo to give 7-4 (59 mg, 80%). MS m/z (M+H) 258.0 found, 279.258.1 required.

### (±) cis-6-oxo-1,2,3,4,6,12a-hexahydropyrido[1,2-b][2]benzazepine-3-carboxylic acid (7-5)

To a solution of (4) cis-3-fluoro-5-oxo-5,7,8,9,10,10a-hexahydrodipyrido[1,2-a:3′,4′-e]azepine-8-carboxylic acid 7-4 (23 mg, 0.09 mmol) in DCM (1.2 mL) was added thionyl chloride (32 μL, 0.45 mmol). The reaction was stirred for 1 hour at RT. To the solution was added toluene (2.0 mL). The mixture was concentrated to give crude 7-5 which was used directly in next step.

(±) cis-(Z)-4-methyl-N′-({[(3S,12aS)-6-oxo-1,2,3,4,6,12a-hexahydropyrido[1,2-b][2]benzazepin-3-yl]carbonyl}oxy)-1H-pyrrole-2-carboximidamide (7-6)

A solution of N′-hydroxy-4-methyl-1H-pyrrole-2-carboximidamide (15.1 mg, 0.11 mmol, prepared as described in WO 2006/123257) in THF (0.5 mL) was cooled to 0° C. To this was added TEA (15 μL, 0.11 mmol) and a solution of (±) cis-3-fluoro-5-oxo-5,7,8,9,10,10a-hexahydrodipyrido[1,2-a:3′,4′-e]azepine-8-carbonyl chloride 7-5 (25 mg, 0.09 mmol) in DCM (0.4 mL). The clear solution was allowed to stir for 30 min. The reaction was partitioned between DCM and water. The organic layer was washed with brine and concentrated to give crude 7-6. m/z (M+H) 379.1 found, 379.2 required.

### (±) cis-(Z)-3-[3-(4-methyl-1H-pyrrol-2-yl)-1,2,4-oxadiazol-5-yl]-1,3,4,12a-tetrahydropyrido[1,2-b][2]benzazepin-6(2H)-one (7-7)

1M TBAF solution in THF (50 μL) was added to a solution of (±) cis-(Z)—N′-((3-fluoro-5-oxo-5,7,8,9,10,10a-hexahydrodipyrido[1,2-a:3′,4′-e]azepine-8-carbonyl)oxy)-4-methyl-1H-pyrrole-2-carboximidamide 7-6 (28 mg, 0.074 mmol) in THF (0.6 mL) at 0° C. The reaction solution was stirred for two hours. The reaction mixture was purified by preparative thin layer silica gel chromatography (50% EtOAc in hexane) to give cis racemate 7-7. MS m/z (M+H) 361.1 found, 361.2 required.

### Example 10

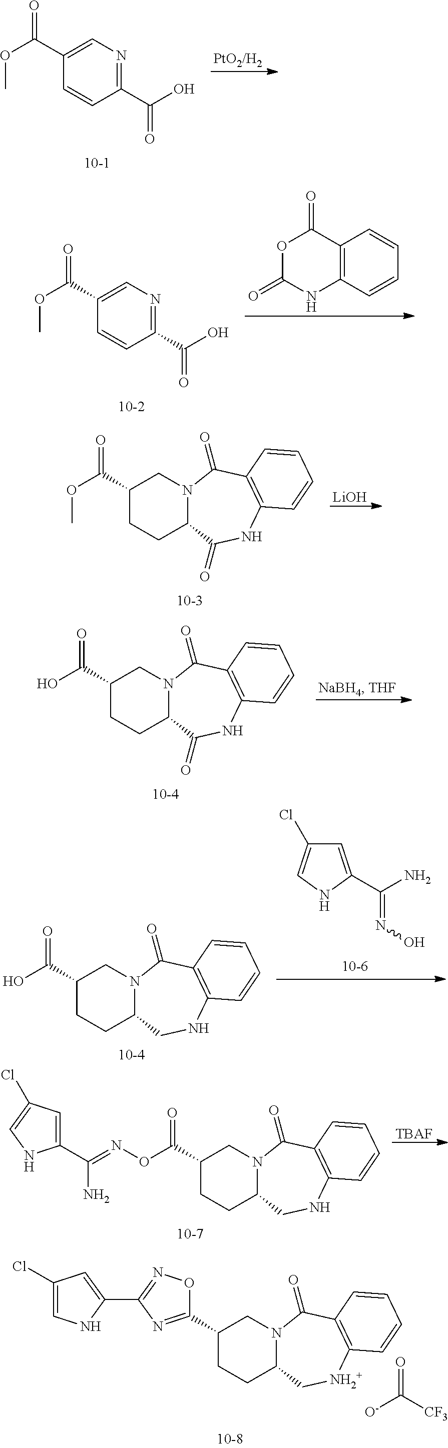

### (2S,5S)-5-(methoxycarbonyl)piperidine-2-carboxylic acid (10-2)

A suspension of 5-(methoxycarbonyl)pyridine-2-carboxylic acid 10-1 (5.00 g, 27.6 mmol) and PtO₂ (1.00 g, 4.42 mmol) in methanol (100 mL) was evacuated and purged with Ar three times. The reaction was evacuated and purged with H₂ and maintained at 45 psi with vigorous shaking. After 4 hours, the reaction was evacuated and purged with N₂. The mixture was filtered under N₂ through a pad of diatomaceous earth washing with aqueous methanol. The filtrate was concentrated to yield crude 10-2 which was used directly in the next step. MS m/z (M+H) 188.5 found, 188.2 required.

### methyl (6aS,9S)-6,12-dioxo-5,6,6a,7,8,9,10,12-octahydropyrido[2,1-c][1,4]benzodiazepine-9-carboxylate (10-3)

10-2 (535 mg, 2.86 mmol) and isatoic anhydride (466 mg, 2.86 mmol) were suspended in DMSO (10 mL) and placed in an oil bath at 120° C. under N₂. After 40 hours, the reaction was diluted with ethyl acetate (100 mL) and washed with water (3×50 mL). The aqueous was back extracted with ethyl acetate (2×50 mL) and the combined organic extracts were washed with water (3×25 mL). The extracts were dried (MgSO₄), filtered and concentrated. This was purified by silica gel chromatography eluting with 0-75% ethyl acetate/hexanes to afford 10-3. MS m/z (M+H) 289.4 found, 289.3 required.

### (6aS,9S)-6,12-dioxo-5,6,6a,7,8,9,10,12-octahydropyrido[2,1-c][1,4]benzodiazepine-9-carboxylic acid (10-4)

To 10-3 (400 mg, 1.387 mmol) suspended in methanol (10 mL) was added 1N LiOH (4.16 mL, 4.16 mmol) and the reaction was placed in an oil bath at 40° C. After 1 h, the reaction was allowed to cool to RT and 1N HCl (10 mL) was added to the mixture which was then concentrated to remove the organic solvent. This was extracted with ethyl acetate (100 mL) and the extract was washed with water (2×50 mL), dried (MgSO₄), filtered and the solvent was evaporated under reduced pressure to yield 10-4. MS m/z (M+H) 275.1029 found, 275.1026 required.

### (6aS,9S)-12-oxo-5,6,6a,7,8,9,10,12-octahydropyrido[2,1-c][1,4]benzodiazepine-9-carboxylic acid (10-5)

10-4 (50 mg, 0.182 mmol) and NaBH₄ (20.69 mg, 0.547 mmol) were suspended in DME (1 mL) and cooled to 0° C. Trifluoroacetic acid (0.053 mL, 0.684 mmol) was added slowly under N₂ and allowed to warm to RT. After 5 hours at RT, the reaction was quenched with methanol and the mixture was purified by reverse phase chromatography (Phenomenex Axia C18(2) 100 A 10 u), eluting with 5-95% MeCN/H₂O+0.1% TFA. Pure product fraction was lyophilized to give 10-4. MS m/z (M+H) 261.0 found, 261.3 required.

### 4-chloro-N′-({[(6aS,9S)-12-oxo-5,6,6a,7,8,9,10,12-octahydropyrido[2,1-c][1,4-]benzodiazepin-9-yl]carbonyl}oxy)-1H-pyrrole-2-carboximidamide (10-7)

To mixture of EDC (14.58 mg, 0.076 mmol) and HOBT (10.28 mg, 0.076 mmol) as solids was added 10-5 (18 mg, 0.069 mmol) in NMP (1 mL). Upon achieving a solution, 4-chloro-N′-hydroxy-1H-pyrrole-2-carboximidamide 10-6 (13.79 mg, 0.086 mmol, prepared as described in WO 2006/123257) was added as a solid. After 30 minutes, the reaction was diluted with ethyl acetate (25 mL), washed with water (3×10 mL), dried (MgSO₄), filtered and the solvent was evaporated under reduced pressure to afford crude 10-7 that was used in the next step without purification. MS m/z (M+H) 402.0 found, 402.9 required.

### (6aS,9S)-9-[3-(4-chloro-1H-pyrrol-2-yl)-1,2,4-oxadiazol-5-yl]-12-oxo-5,6,6a,7,8,9,10,12-octahydropyrido[2,1-c][1,4]benzodiazepin-5-ium trifluoroacetate (10-8)

10-7 (26 mg, 0.065 mmol) was dissolved in THF (1 ml) under N₂. To this at RT was added 1M TBAF in THF (0.065 mL, 0.065 mmol). 4A sieves (100 mg) were added and the reaction was placed in an oil bath at 40° C. After 2 hours at this temperature, the reaction was diluted with ethyl acetate, filtered and washed with water. The organic layer was dried (MgSO₄), filtered and the solvent was evaporated under reduced pressure. The resulting residue was purified by reverse phase chromatography (Phenomenex Axia C18(2) 100 A 10 u) eluting with 5-95% MeCN/H₂O+0.1% TFA. Product fraction was lyophilized to yield 10-8 as a racemate. MS m/z (M+H) 384.1221 found, 384.1222 required.

### Example 11

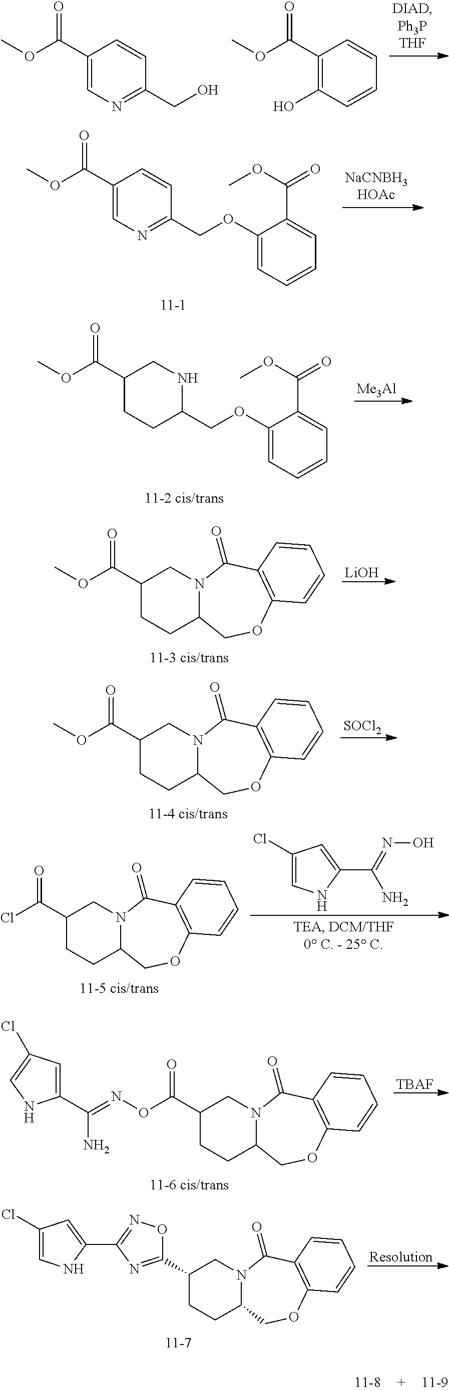

### Methyl 6-{[2-(methoxycarbonyl)phenoxy]methyl}pyridine-3-carboxylate (11-1)

A solution of methyl 6-(hydroxymethyl)pyridine-3-carboxylate (5.0 g, 29.9 mmol) in THF (80 ml) was treated with methyl salicyclate (4.25 mL, 32.9 mmol) and triphenylphosphine (8.63 g, 32.9 mmol) and the resulting solution was cooled over an ice bath for 5 minutes and then treated with diisopropylazodicarboxylate (6.40 mL, 32.9 mmol) and then stirred cold for 30 minutes and allowed to warmed to 25° C. and stirred for an additional 60 minutes. After this time, the mixture was partitioned between CH₂Cl₂ (100 mL) and water (75 mL). The organic extract was dried over MgSO₄, filtered and concentrated in vacuo. This residue was purified by column chromatography using a Redi Sep column (330 g) and eluting with a gradient of 0-25% EtOAc/CH₂Cl₂. The desired fractions were combined and concentrated in vacuo to provide 11-1. m/z (M+H) 302.0 found, 302.1 required.

### cis/trans-Methyl 6-{[2-(methoxycarbonyl)phenoxy]methyl}piperidine-3-carboxylate (11-2)

A solution of 11-1 (8.19 g, 27.2 mmol) was dissolved in acetic acid (80 mL) and cooled over ice bath. This solution was treated with NaCNBH₃ (10.4 g, 166 mmol) portionwise over 15 minutes. The cooling bath was removed and the mixture was stirred for 2 hours at 25° C. This mixture was diluted with EtOAc (600 mL) and quenched with crushed ice (200 g). This mixture was treated with Na₂CO₃ to basic pH and the resulting layers were partitioned and the aqueous was further extracted with EtOAc (3×750 mL). The combined organic extracts were dried over MgSO₄, filtered and evaporated in vacuo. This residue was purified by column chromatography using a RediSep column (330 g) eluting with a gradient of 0-10% aq NH₄OH/CH₃CN. The desired fractions were combined to give 11-2. m/z (M+H) 308.0 found, 308.1 required.

### cis/trans-Methyl 12-oxo-6,6a,7,8,9,10-hexahydro-12H-pyrido[2,1-c][1,4]benzoxazepine-9-carboxylate (11-3)

A solution of 11-2 (1.74 g, 5.66 mmol) in CH₂Cl₂ (100 mL) was cooled over a dry ice/acetone bath and treated with 2M Me₃Al (2.83 ml, 5.66 mmol) and then stirred at 25° C. for 48 days. After this time, the reaction was quenched with crushed ice (50 g) and treated with 1N HCl to pH=1. This mixture was partitioned and the aqueous layer back extracted with CH₂Cl₂ (200 mL). The combined extracts were dried over MgSO₄, filtered and the solvent evaporated in vacuo. This residue was purified by column chromatography using RediSep column (120 g) eluting with a gradient of 0-20% EtOAc/CH₂Cl₂ to give 11-3. m/z (M+H) 276.1 found, 276.1 required.

### cis/trans-12-oxo-6,6a,7,8,9,10-Hexahydro-12H-pyrido[2,1-c][1,4]benzoxazepine-9-carboxylic acid (11-4)

A solution of 11-3 (698 mg, 2.54 mmol) in MeOH (10 mL) was treated with 1N LiOH (3.80 mL, 3.80 mmol) and stirred at 25° C. for 1.5 hours. This solution was concentrated in vacuo to about 4 mL volume and treated with 1N HCl (5 mL). This mixture was extracted with CH₂Cl₂ (2×12 mL). The combined extracts were washed with water (2 mL), dried over MgSO₄, filtered and evaporated in vacuo to give 11-4. m/z (M+H) 262.0 found, 262.1 required.

### cis/trans-12-oxo-6,6a,7,8,9,10-Hexahydro-12H-pyrido[2,1-c][1,4]benzoxazepine-9-carbonyl chloride (11-5)

A solution of 11-4 (808 mg, 3.09 mmol) in CH₂Cl₂ (20 mL) was treated with thionyl chloride (1.129 mL, 15.5 mmol) and stirred at 25° C. for 30 minutes. After this time, reaction mixture was evap in vacuo and reevaporated from toluene (5 mL) and dried under high vacuum to give 11-5. This material was used without further purification.

### cis/trans-4-Chloro-N′-{[(12-oxo-6,6a,7,8,9,10-hexahydro-12H-pyrido[2,1-e][1,4]benzoxazepin-9-yl)carbonyl]oxy}-1H-pyrrole-2-carboximidamide (I 1-6)

A suspension of 4-chloro-N′-hydroxy-1H-pyrrole-2-carboximidamide (396 mg, 2.48 mmol) in THF (4 mL) was treated with TEA (346 μL, 2.48 mmol) and stirred until mostly dissolved. This mixture was cooled over ice bath and treated with a solution of 11-5 (694 mg, 2.48 mmol) in CH₂Cl₂ (4 ml) slowly over 5 minutes. Stirred over ice bath for 10 minutes and then stirred at 25° C. for 30 minutes. This mixture was diluted with water (7 ml) and extracted with CH₂Cl₂ (2×12 ml). The combined extracts were dried over MgSO₄, filtered and evaporated in vacuo to obtain compound 11-6. This crude product was used without further purification. m/z (M+H) 403.0 found, 403.1 required.

### (6aS,9S)-9-[3-(4-chloro-1H-pyrrol-2-yl)-1,2,4-oxadiazol-5-yl]-6,6a,7,8,9,10-hexahydro-12H-pyrido[2,1-c][1,4]benzoxazepin-12-one and (6aR,9R)-9-[3-(4-chloro-1H-pyrrol-2-yl)-1,2,4-oxadiazol-5-yl]-6,6a,7,8,9,10-hexahydro-12H-pyrido[2,1-c][1,4]benzoxazepin-12-one(11-8 and 11-9)

A solution of 11-6 (1.2 g, 2.48 mmol) in THF (2.5 ml) was treated with 1M TBAF (2.5 mL, 2.5 mmol) and warmed to 40° C. for 20 hours and monitored by LC-MS. After this time, added more 1M TBAF (2.5 ml, 2.5 mmol) and heated to 40° C. for an additional 18 hours. This mixture was cooled to 25° C. and diluted with water (25 mL) and extracted with EtOAc (2×50 ml). The combined organic extracts were dried over MgSO₄, filtered and the solvent was evaporated in vacuo. This residue was purified by column chromotagraphy using RediSep column (120 g) and eluting with a gradient of 0-20% EtOAc/CH₂Cl₂. The lower Rf isomer fractions were combined and evaporated in vacuo to give racemic 11-7. MS m/z (M+1) 385.0 found, 384.1 required. Pure enantiomers 11-8 and 11-9 were obtained from chiral HPLC separation of racemic 4-7. 11-8 NMR (CDCl₃) δ=8.84 (br s, 1H), 7.93 (d, 1H, J=7.8 Hz), 7.42 (t, 1H, J=7 Hz), 7.19 (t, 1H, J=7.5 Hz), 7.02 (d, 1H, J=8 Hz), 6.85 (m, 1H), 6.79 (m, 1H), 4.54-4.42 (m, 2H), 4.14 (m, 1H), 3.96 (m, 2H), 3.40 (m, 1H) 2.20-2.04 (m, 2H) and 2.00-1.83 (m, 2H) ppm. MS m/z (M+1) 385.1064 found, 385.1062 required.

### Example 12

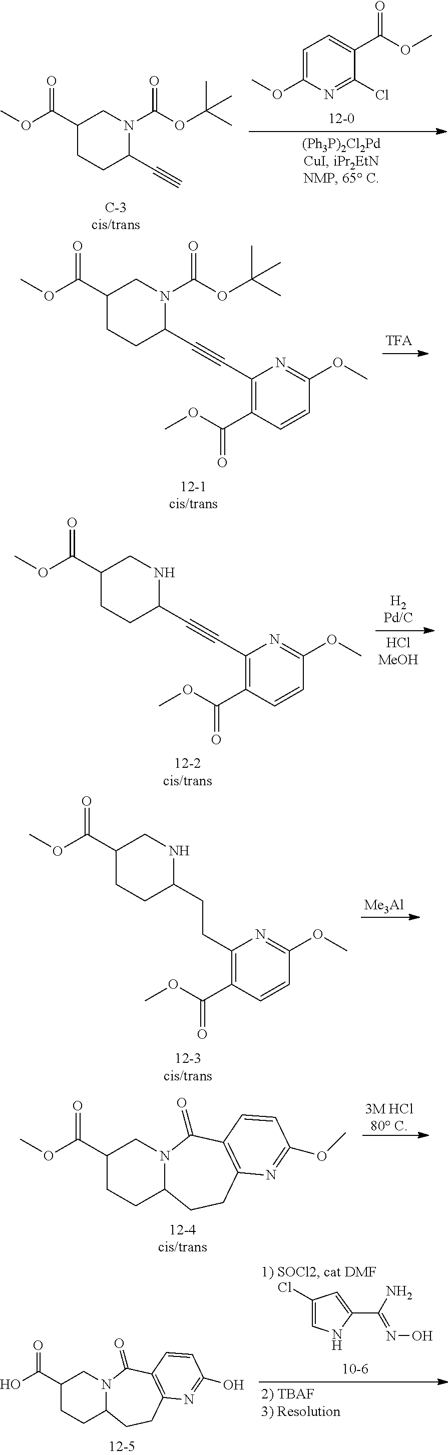

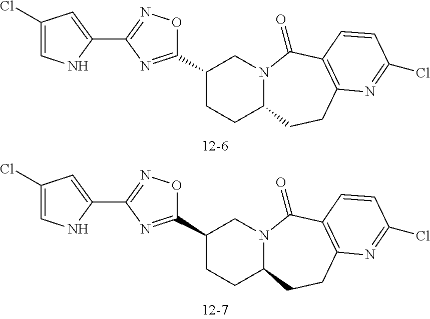 [SMILES: O=C1c2ccc(Cl)nc2CC[C@@H]2CC[C@H](c3nc(-c4cc(Cl)c[nH]4)no3)CN12 ; O=C1c2ccc(Cl)nc2CC[C@H]2CC[C@@H](c3nc(-c4cc(Cl)c[nH]4)no3)CN12]

### cis/trans 1-tert-butyl 3-methyl 6-{[6-methoxy-3-(methoxycarbonyl)pyridin-2-yl]ethynyl}piperidine-1,3-dicarboxylate (12-1)

A mixture of C-3 (1.19 g, 4.46 mmol), 12-0 (Chem. Pharm. Bull. 2000, 48, 1847) (900 mg, 4.46 mmol), bis(triphenylphosphine)palladium(II)chloride (157 mg, 0.223 mmol) and diisopropylethylamine (1.559 ml, 8.93 mmol) in NMP (20 ml) and degassed under high vac. The resulting mixture was treated with CuI 68 mg, 0.357 mmol) and heated to 70° C. After 18 hours, the mixture was diluted with water (200 ml) and extracted with EtOAc (2×200 ml). The combined extracts were washed with water (100 ml), dried over MgSO4, filtered and evap the solvent in vacuo. This residue was purified by chromatography using silica gel (40 g) and eluting with a 0-20% gradient of EtOAc/CH₂Cl₂ to give 12-1. m/z (M+H) 433.0 found, 433.19 required.

### cis/trans Methyl 6-methoxy-2-{[5-(methoxycarbonyl)piperidin-2-yl]ethynyl}pyridine-3-carboxylate (12-2)

A solution of 12-1 (1.31, 3.04 mmol) in a mixture of TFA and CH₂Cl₂ (8 ml) was allowed to stand at 25° C. After 30 minutes, the solvent was evaporated in vacuo. This residue was redissolved in CH₂Cl₂ and reevaporated in vacuo. The resulting residue was dried under high vac for 18 hours to give 12-2. m/z (M+H) 333.0 found, 333.1 required.

### Cis/trans Methyl 6-methoxy-2-{2-[5-(methoxycarbonyl)piperidin-2-yl]ethyl}pyridine-3-carboxylate (12-3)

To a degassed solution of 12-2 (1.70 g, 3.04 mmol) in MeOH (50 ml) containing conc. HCl (507 ul, 6.08 mmol) was added 10% Pd/C (170 mg) and shook under 47 psi H₂. After 3 days, the reaction was monitored by LC-MS and showed no desired product. This mixture was filtered through celite and the solvent was evaporated in vacuo. This residue was redissolved in MeOH 50 ml) with cone HCL (507 ul, 6.08 mmol), degassed, treated with 10% Pd/C (340 mg) and shook under 47 psi. H₂ for 18 hours. This mixture was filtered through celite and the pad was rinsed with MeOH (50 ml). The solvent from the filtrate was evaporated in vacuo. This residue was partitioned between sat. aq. Na₂CO₃ (75 ml) and CH₂Cl₂ (2×50 ml). The combined extracts were concentrated in vacuo and the residue was purified by chromatography using silica gel (40 g) and eluting with a 0-10% gradient of NH₄OH/ACN to give 12-3 as an oil. m/z (M+H) 337.0 found, 337.2 required.

### Cis/trans Methyl 2-methoxy-5-oxo-5,7,8,9,10,10a,11,12-octahydrodipyrido[1,2-a:2′,3′-e]azepine-8-carboxylate (12-4)

A solution of 12-3 (589 mg, 1.75 mmol) in CH₂Cl₂ (20 ml) was cooled over an ice bath and treated with 3M Me₃Al in toluene (1.75 ml, 3.5 mmol). The cooling bath was removed and the reaction was stirred at 25° C. After 18 hours, the mixture was cooled over an ice bath and carefully quenched with water (20 ml). Treated with 1N HCl to pH=3 and extracted with CH₂Cl₂ (2×50 ml). The combined extracts were dried over MgSO4, filtered and the solvent removed in vacuo. This residue was purified by chromatography using silica gel (40 g) eluting with a 0-30% gradient of EtOAc/CH₂Cl₂ to give 12-3. m/z (M+H) 305.0 found, 305.1 required.

### Cis/trans 2-Hydroxy-5-oxo-5,7,8,9,10,10a,11,12-octahydrodipyridol[1,2-a:2′,3′-e]azepine-8-carboxylic acid (12-5)

A solution of 12-3 (229 mg, 0.75 mmol) in 3M HCl (10 ml) was heated to 80° C. After 5 hours, the mixture was concentrated in vacuo and the residue was purified by reverse phase chromatography using a LUNA column and eluting with a 5-95% aq/ACN (0.1% TFA). The desired fractions were lyophilized to give 12-5. m/z (M+H) 277.0 found, 277.1 required.

### (8S,10aS)-2-chloro-8-[3-(4-chloro-1H-pyrrol-2-yl)-1,2,4-oxadiazol-5-yl]-8,9,10,10a,11,12-hexahydrodipyrido[1,2-a:2′,3′-e]azepin-5(7H)-one (12-6) and (8R,10aR)-2-chloro-8-[3-(4-chloro-1H-pyrrol-2-yl)-1,2,4-oxadiazol-5-yl]-8,9,10,10a,11,12-hexahydrodipyrido[1,2-a:2′,3′-e]azepin-5(7H)-one (12-7)

A suspension of 12-5 (71 mg, 0.257 mmol) in thionyl chloride (2 ml) was treated with DMF (4 ul, 0.05 mmol) and heated to reflux. After 3 hours, the mixture was concentrated in vacuo and the residue was azeotroped from toluene (2 ml) to eliminate excess thionyl chloride. This residue was dissolved in CH₂Cl₂ (1 ml) and cooled over an ice bath. The cooled mixture was treated with a solution of 10-6 (41.5 mg, 0.257 mmol) and TEA (72 ul, 0.514 mmol) in THF (500 ul). The cooling bath was removed and the mixture stirred for another 20 minutes. This mixture was partitioned between CH₂Cl₂ (2×30 ml) and water (10 ml). The combined organic extracts were dried over MgSO₄, filtered and evap in vacuo. This residue was dissolved in THF (2 ml) and treated with 1M TBAF (257 ul, 0.257 mmol) and heated to 60° C. After 18 hours, the reaction was allowed to cool to 25° C. and partitioned between CH₂Cl₂ (2×30 ml) and water (10 ml). The combined organic extracts were dried over MgSO₄, filtered and evap in vacuo. This residue was purified by chromatography using silica gel (12 g) and eluting with a 0-30% gradient of EtOAc/CH₂Cl₂ to give pure cis and pure trans compounds. The cis compound was resolved using an AD-H column eluting with 40% IPA/heptanes to give 12-6 (first eluting) and 12-7 (second eluting) as amorphous solids. NMR (CDCl₃) δ=8.98 (br s, 1H), 7.92 (d, 1H, J=8 Hz), 7.33 (d, 1H, J=8 Hz), 6.92 (m, 1H), 6.87 (m, 1H), 5.02 (d, 1H), 3.70 (m, 1H), 3.18-3.34 (m, 2H), 2.88-3.07 (m, 2H), 2.43 (m, 1H), 2.15 (m, 1H) and 1.72-2.05 (m, 4H) ppm. m/z (M+H) 418.0 found, 418.1 required.

### Example 13

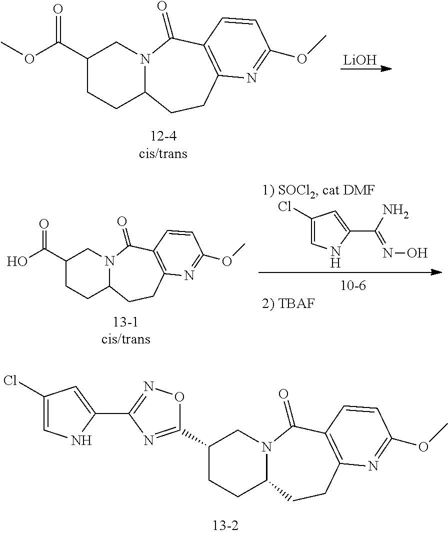

### Cis/trans 2-Methoxy-5-oxo-5,7,8,9,10,10a,11,12-octahydrodipyrido[1,2-a:2′,3′-e]azepine-8-carboxylic acid (13-1)

To a solution of 12-4 (23.8, 0.078 mmol) in MeOH (500 ul) was added 1N LiOH (117 ul, 0.117 mmol) and stirred at 25° C. After 1.5 hours, the reaction mixture was treated with 1N HCl (117 ul, 0.117 mmol) and water (1 ml) and extracted with CH₂Cl₂ (2×2 ml). The combined extracts were dried over MgSO₄, filtered and evap in vacuo to give 13-1. m/z (M+H) 291.0 found, 291.1 required.

### (8S,10aS)-8-[3-(4-chloro-1H-pyrrol-2-yl)-1,2,4-oxadiazol-5-yl]-2-methoxy-8,9,10,10a,11,12-hexahydrodipyrido[1,2-a:2′,3′-e]azepin-5(7H)-one (13-2)

A solution of 13-1 (21.7 mg, 0.075 mmol) in CH2Cl2 (1 ml) was treated with thionyl chloride (27 ul, 0.374 mmol) and stirred at 25° C. After 30 minutes, the mixture was concentrated in vacuo and the residue was azeotroped from toluene (1 ml) to eliminate excess thionyl chloride. This residue was dissolved in CH₂Cl₂ (500 ul) and cooled over an ice bath. The cooled mixture was treated with a solution of 10-6 (11.9 mg, 0.075 mmol) and TEA (13 ul, 0.075 mmol) in THF (500 ul). The cooling bath was removed and the mixture stirred for another 20 minutes. This mixture was partitioned between CH₂Cl₂ (2×2 ml) and water (1 ml). The combined organic extracts were dried over MgSO₄, filtered and evap in vacuo. This residue was dissolved in THF (500 ul) and treated with 1M TBAF (75 ul, 0.075 mmol) and stirred at 25° C. After 3 days, the reaction was partitioned between CH₂Cl₂ (2×5 ml) and water (3 ml). The combined organic extracts were dried over MgSO4, filtered and evaporated in vacuo. This residue was purified by chromatography using silica gel (12 g) and eluting with a 0-40% gradient of EtOAc/CH₂Cl₂ to give pure cis 13-2 as an amorphous solid. NMR (CDCl₃) δ=8.96 (br s, 1H), 7.84 (d, 1H, J=8.4 Hz), 6.91 (m, 1H), 6.87 (m, 1H), 6.60 (d, 1H, J=4 Hz), 5.04 (d, 1H, J=9 Hz), 4.12 (m, 1H), 3.97 (s, 3H), 3.78 (m, 1H), 3.20-3.35 (m, 2H), 2.82-2.93 (m, 2H), 2.40 (m, 1H), and 1.8-2.0 (m, 4H) ppm. MS m/z (M+H) 414.1321 found, 414.1255 required.

### Example 14

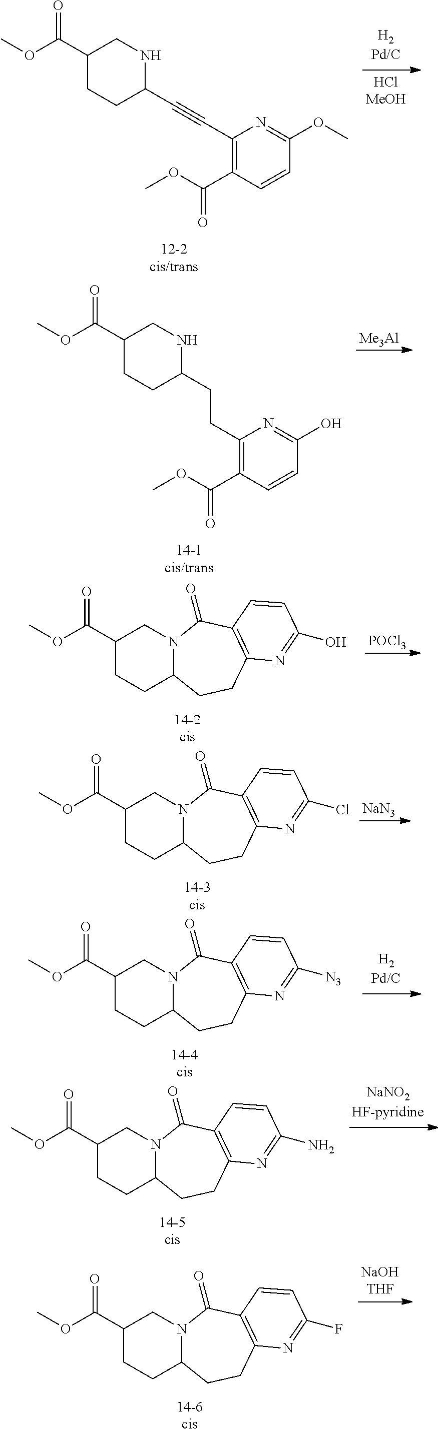

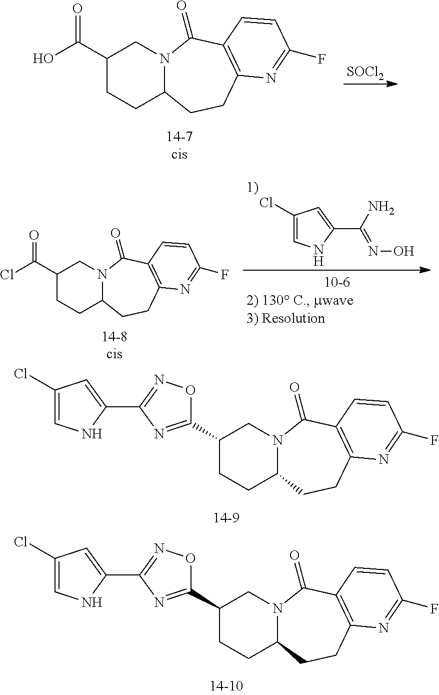

### Cis/trans-Methyl 6-hydroxy-2-{2-[5-(methoxycarbonyl)piperidin-2-yl]ethyl}pyridine-3-carboxylate (14-1)

To a degassed solution of 12-2 (7.60 g, 18.7 mmol) in MeOH (200 mL) containing cone. HCl (9.38 mL, 2.0 M in dioxane) was added 10% Pd/C (760 mg) and shook under 47 psi H₂ for 43 h. After 18 h, additional 10% Pd/C (760 mg) was added and after 41 h additional cone. HCl (9.38 mL, 2M in dioxane) was added. This mixture was filtered through celite and the pad was rinsed with MeOH (100 mL). The solvent from the filtrate was evaporated in vacuo and the residue was partitioned between sat. aq. Na₂CO₃ (100 mL) and CH₂Cl₂ (200 mL). The aqueous was further extracted with CH₂Cl₂ (100 mL) and the combined extracts were dried over MgSO₄, filtered, and concentrated in vacuo. The residue was purified by chromatography using silica gel (330 g) and eluting with a 0-10% gradient of NH₄OH/ACN to provide 14-1.

### Cis-Methyl 2-hydroxy-5-oxo-5,7,8,9,10,10a,11,12-octahydrodipyrido[1,2-a:2′,3′-e]azepine-8-carboxylate (14-2)

A solution of 14-1 (2.25 g, 6.98 mmol) in CH₂Cl₂ (50 mL) was to −78° C. and treated with 2M Me₃Al in toluene (6.98 mL, 14.0 mmol). The cooling bath was removed and the reaction was stirred at 25° C. After 18 hours, the mixture was cooled to 0° C. and carefully quenched with ice water (20 mL). The reaction mixture was poured onto 1N HCl (100 mL) and extracted with CH₂Cl₂ (3×50 mL). The combined extracts were dried over Na₂SO₄, filtered, and concentrated in vacuo. The residue was purified by chromatography using silica gel (120 g) and eluting with a 0-10% gradient of MeOH/CH₂Cl₂ to provide 14-2. m/z (M+H) 291.0 found, 291.1 required.

### Cis-Methyl 2-chloro-5-oxo-5,7,8,9,10,10a,11,12-octahydrodipyrido[1,2-a:2′,3′-e]azepine-8-carboxylate (14-3)

A solution of 14-2 (598 mg, 2.06 mmol) and POCl₃ (0.576 mL, 6.18 mmol) in ACN (6 mL) was heated to 100° C. After 4 hours, the mixture was poured onto sat. aq. NaHCO₃ (20 mL) and extracted with CH₂Cl₂ (3×20 mL). The combined extracts were dried over Na₂SO₄, filtered, and concentrated in vacuo to provide 14-3. m/z (M+H) 309.0 found, 309.1 required.

### Cis-Methyl 2-azido-5-oxo-5,7,8,9,10,10a,11,12-octahydrodipyrido[1,2-a:2′,3′-e]azepine-8-carboxylate (14-4)

A solution of 14-3 (597 mg, 1.93 mmol) and NaN₃ (189 mg, 2.90 mmol) in DMSO (10 mL) was heated to 120° C. After 21 hours, the mixture was poured onto water (30 mL) and extracted with EtOAc (7×20 mL). The combined extracts were washed with water (10 mL), dried over Na₂SO₄, filtered, and concentrated in vacuo to provide 14-4. m/z (M+H) 316.0 found, 316.1 required.

### Cis-Methy 2-amino-5-oxo-5,7,8,9,10,10a,11,12-octahydrodipyrido[1,2-a:2′,3′-e]azepine-8-carboxylate (14-5)

To a degassed solution of 14-4 (540 mg, 1.71 mmol) in MeOH (10 mL) and EtOAc (10 mL) was added 10% Pd/C (150 mg) and stirred under a balloon of H₂ for 18 h. This mixture was filtered through celite and the pad was rinsed with MeOH (3×10 mL). The solvent from the filtrate was evaporated in vacuo to provide 14-5. m/z (M+H) 290.0 found, 290.1 required.

### Cis-Methyl 2-fluoro-5-oxo-5,7,8,9,10,10a,11,12-octahydrodipyrido[1,2-a:2′,3′-e]azepine-8-carboxylate (14-6)

To a solution of 14-5 (458 mg, 1.58 mmol) in 70% hydrogen fluoride-pyridine (5 mL) at 0° C. was added sodium nitrite (218 mg, 3.17 mmol) in three portions. The reaction mixture was warmed to 25° C. for 10 min, then poured onto water (30 mL), which was neutralized with the slow addition of NaHCO₃. The mixture was extracted with CH₂Cl₂ (3×20 mL). The combined extracts were dried over Na₂SO₄, filtered, and concentrated in vacuo to provide 14-6. m/z (M+H) 293.0 found, 293.1 required.

### Cis-2-Fluoro-5-oxo-5,7,8,9,10,10a,11,12-octahydrodipyrido[1,2-a:2′,3′-e]azepine-8-carboxylic acid (14-7)

To a solution of 14-6 (276 mg, 0.944 mmol) in THF (9 mL) was added 1.0N NaOH (0.944 mL, 0.944 mmol) and stirred at 25° C. for 2 h. The solution was poured onto 1.0N HCl (20 mL) and extracted with EtOAc (3×10 mL). The combined extracts were dried over Na₂SO₄, filtered, and concentrated in vacuo to provide 14-7. MS m/z (M+H) 279.0 found, 279.1 required.

### Cis-2-Fluoro-5-oxo-5,7,8,9,10,10a,11,12-octahydrodipyrido[1,2-a:2′,3′-e]azepine-8-carbonyl chloride (14-8)

To a solution of 14-7 (137 mg, 0.492 mmol) in CH₂Cl₂ (3 mL) was added thionyl chloride (0.180 mL, 2.46 mmol). The reaction was stirred for 30 min at RT. The solution was diluted with toluene (3 mL) and concentrated to provide crude 14-8, which was used directly in next step.

### (8S,10aS)-8-[3-(4-Chloro-1H-pyrrol-2-yl)-1,2,4-oxadiazol-5-yl]-2-fluoro-8,9,10,10a,11,12-hexahydrodipyrido[1,2-a:2′,3′-e]azepin-5(7H)-one (14-9)

A solution of N′-hydroxy-4-chloro-1H-pyrrole-2-carboximidamide 10-6 (63.2 mg, 0.396 mmol, prepared as described in WO 2006/123257) in THF (1 mL) and CH₂Cl₂ (1 mL) was cooled to 0° C. To this was added TEA (0.105 mL, 0.755 mmol) and a solution of 14-8 (112 mg, 0.377 mmol) in CH₂Cl₂ (1 L). The solution was allowed to stir for 30 min. The reaction was poured onto sat, aq. NaHCO₃ (10 mL) and extracted with CH₂Cl₂ (3×10 mL). The combined extracts were dried over Na₂SO₄, filtered, and concentrated in mow. The crude intermediate was dissolved in DMA (2 mL) and heated in a Biotage microwave reactor at 130° C. for 20 min, then cooled and concentrated in vacuo. The crude product was purified by chromatography using silica gel (40 g) and eluting with a 2-100% gradient of EtOAc/CH₂Cl₂ to give cis racemate. The single enantiomers 14-9 and 14-10 were obtained from chiral HPLC separation of racemate, Preparative HPLC was performed on a ChiralCel OD-H column (2 cm×25 cm, eluted with 60:40 CO₂:MeOH). 14-9 (peak one): MS m/z (MSH) 402.1124 found, 402.1128 required. 14-10 (peak two): MS m/z (M+H) 402.1124 found, 402.1128 required.

### Example 15

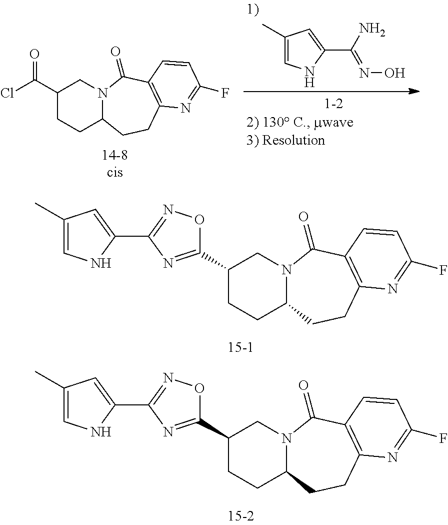

### (8S,10aS)-2-Fluoro-8-[3-(4-methyl-1H-pyrrol-2-yl)-1,2,4-oxadiazol-5-yl]-8,9,10,10a,11,12-hexahydrodipyrido[1,2-a:2′,3′-e]azepin-5(7H)-one (15-1)

A solution of N′-hydroxy-4-methyl-1H-pyrrole-2-carboximidamide 1-2 (69.9 mg, 0.502 mmol, prepared as described in WO 2006/123257) in THF (2 mL) and CH₂Cl₂ (2 mL) was cooled to 0° C. To this was added TEA (0.140 mL, 1.00 mmol) and a solution of 14-8 (149 mg, 0.502 mmol) in CH₂Cl₂ (1 mL). The solution was allowed to stir for 1 h. The reaction was poured onto sat. aq. NaHCO₃ (10 mL) and extracted with CH₂Cl₂ (3×10 mL). The combined extracts were dried over Na₂SO₄, filtered, and concentrated in vacuo. The crude intermediate was dissolved in DMA (4 mL) and heated in a Biotage microwave reactor at 130° C. for 20 min, then cooled and concentrated in vacuo. The crude product was purified by chromatography using silica gel (40 g) and eluting with a 1-100% gradient of EtOAc/CH₂Cl₂ to give cis racemate. The single enantiomers 15-1 and 15-2 were obtained from chiral HPLC separation of racemate. Preparative HPLC was performed on a ChiralCel OD-H column [2 cm×25 cm, eluted with 80:20 Heptanes:EtOH (containing 0.1% TEA)]. 15-1 (peak one): MS m/z (M+H) 382.1669 found, 382.1674 required. 15-2 (peak two): MS m/z (M+H) 382.1667 found 382.1674 required.

### Example 16

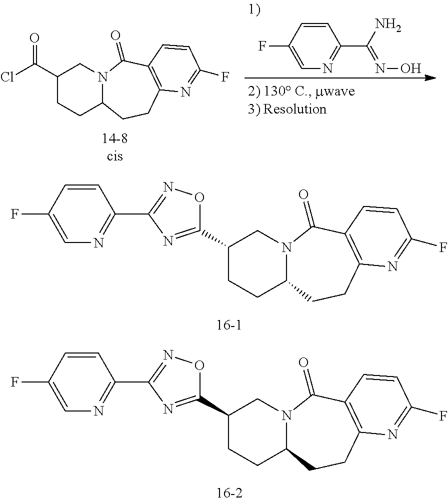 [SMILES: O=C(Cl)C1CCC2CCc3nc(F)ccc3C(=O)N2C1 ; N/C(=N\O)c1ccc(F)cn1 ; O=C1c2ccc(F)nc2CC[C@@H]2CC[C@H](c3nc(-c4ccc(F)cn4)no3)CN12 ; O=C1c2ccc(F)nc2CC[C@H]2CC[C@@H](c3nc(-c4ccc(F)cn4)no3)CN12]

### (8S,10aS)-2-Fluoro-8-[3-(5-fluoropyridin-2-yl)-1,2,4-oxadiazol-5-yl]-8,9,10,10a,11,12-hexahydrodipyrido[1,2-a:2′,3′-e]azepin-5(7H)-one (16-1)

A solution of 5-fluoro-N′-hydroxypyridine-2-carboximidamide (76.0 mg, 0.492 mmol, prepared as described in WO 2002/068417) in THF (1 mL) and CH₂Cl₂ (1 mL) was cooled to 0° C. To this was added TEA (0.137 mL, 0.984 mmol) and a solution of 14-8 (146 mg, 0.492 mmol) in CH₂Cl₂ (1 mL). The solution was allowed to stir for 30 min. The reaction was poured onto sat. aq. NaHCO₃ (10 mL) and extracted with CH₂Cl₂ (3×10 mL). The combined extracts were dried over Na₂SO₄, filtered, and concentrated in vacuo. The crude intermediate was dissolved in DMA (2 mL) and heated in a Biotage microwave reactor at 130° C. for 20 min, then cooled and concentrated in vacuo. The crude product was purified by chromatography using silica gel (40 g) and eluting with a 2-100% gradient of EtOAc/CH₂Cl₂ to give cis racemate. The single enantiomers 16-1 and 16-2 were obtained from chiral HPLC separation of racemate. Preparative HPLC was performed on a ChiralCel OD column [2 cm×25 cm, eluted with EtOH (containing 0.1% TFA)]. 16-1 (peak one): MS m/z (M+H) 398.1424 found, 398.1423 required. 16-2 (peak two): MS m/z (M+H) 398.1425 found, 398.1423 required.

### Example 17

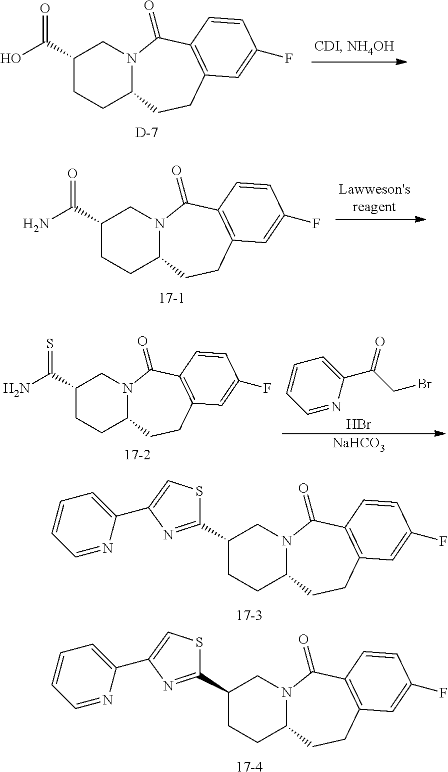

### (3S,12aS)-9-Fluoro-6-oxo-1,2,3,4,6,11,12,12a-octahydropyrido[1,2-b][2]benzazepine-3-carboxylic acid (17-1)

To a suspension of D-7 (3.05 g, 11.0 mmol) in ACN (35 mL) was added carbonyl diimidazole (2.69 g, 16.6 mmol) and stirred for 20 min as the suspension dissolved. This solution was treated with sat, aq. ammonium hydroxide (11.2 mL, 165 mmol) and stirred for 1 h. This mixture was poured onto 1N HCl (100 mL) and extracted with 5% CH₂Cl₂/MeOH (6×50 mL). The combined extracts were dried over Na₂SO₄, filtered and the solvent removed in vacuo to provide 17-1 as a solid. MS m/z (M+H) 277.1 found, 277.1 required.

### (3S,12aS)-9-Fluoro-6-oxo-1,2,3,4,6,11,12,12a-octahydropyrido[1,2-b][2]benzazepine-3-carbothioamide (17-2)

A solution of 17-1 (2.96 g, 10.7 mmol) and Lawweson's reagent (2.38 g, 5.89 mmol) in THF (50 mL) was stirred for 18 h at ambient temperature. The mixture was concentrated in vacuo. The residue was purified by chromatography using silica gel (120 g) and eluting with a 2-100% gradient of EtOAc/CH₂Cl₂ to provide 17-2 as a solid. MS m/z (M+H) 293.0 found, 293.1 required.

### (3S,12aS)-9-Fluoro-3-[4-(pyridin-2-yl)-1,3-thiazol-2-yl]-1,3,4,11,12,12a-hexahydropyrido[1,2-b][2]benzazepin-6(2H)-one (17-3) and (3R,12aS)-9-Fluoro-3-[4-(pyridin-2-yl)-1,3-thiazol-2-yl]-1,3,4,11,12,12a-hexahydropyrido[1,2-b][2]benzazepin-6(2H)-one (17-4)

To a suspension of 2-(bromoacetyl)pyridine hydrobromide (1.15 g, 4.10 mmol) and sodium bicarbonate (0.575 g, 6.84 mmol) in EtOH (25 mL) was added 17-2 (1.00 g, 3.42 mmol). The solution was allowed to stir for 18 h at ambient temperature and then concentrated to a volume of ˜5 mL. The reaction was poured onto water (50 mL) and extracted with CH₂Cl₂ (3×40 mL). The combined extracts were dried over Na₂SO₄, filtered, and concentrated in vacuo. The crude product was purified by chromatography using silica gel (120 g) and eluting with a 5-100% gradient of EtOAc/Hexanes to provide 17-3: MS m/z (M+H) 394.1387 found, 394.1384 required; and 17-4: MS m/z (M+H) 394.1386 found, 394.1384 required.

### Example 18

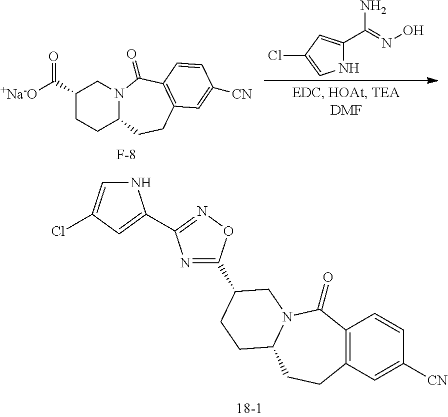

### (±) cis-9-cyano-3-[3-(4-chloro1H-pyrrol-2-yl)-1,2,4-oxadiazol-5-yl]-1,3,4,11,12,12a-hexahydropyrido[1,2-b][2]benzazepin-6(2H)-one (18-1)

To a solution of intermediate A-8 (0.019 g, 0.064 mmol) in DMF (0.2 mL) was added EDC (0.018 g, 0.095 mmol), HOAt (0.013 g, 0.095 mmol) and TEA (0.044 mL, 0.318 mmol) and the reaction was stirred for 15 minutes followed by the addition of N′-hydroxy-4-20 chloro-1H-pyrrole-2-carboximidamide (10-6, 0.010 g, 0.064 mmol). The reaction was stirred at 65° C. overnight. The reaction contents were cooled to ambient temperature and partitioned between EtOAc and ice water. The organic phase washed with brine, dried over MgSO₄, filtered and concentrated. The crude residue was purified by silica gel chromatography (0-100% EtOAc in hexanes) to give 18-1 as a yellow powder. MS m/z (M+H) 408.1216 found, 408.1222 required.

### Example 19

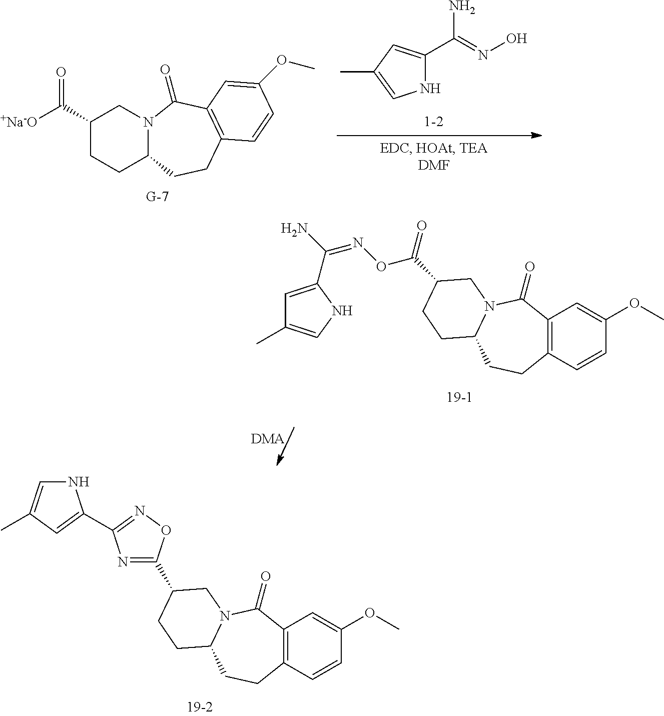

### (±) N′-((cis-10-methoxy-6-oxo-1,2,3,4,6,11,12,12a-Octahydrobenzo[e]pyrido[1,2-a]azepine-3-carbonyl)oxy)-4-methyl-1H-pyrrole-2-carboximidamide (19-1)

To a solution of intermediate G-7 (0.024 g, 0.077 mmol) in DMF (0.25 mL) was added EDC (0.022 g, 0.116 mmol), HOAt (0.015 g, 0.116 mmol) and TEA (0.053 mL, 0.385 mmol) and the reaction was stirred for 15 minutes followed by the addition of N′-hydroxy-4-methyl-1H-pyrrole-2-carboximidamide (1-2, 0.010 g, 0.077 mmol). The reaction was stirred at ambient temperature overnight. The reaction contents were partitioned between EtOAc and ice water and the organic phase was washed with brine, dried over MgSO₄, filtered and concentrated. The crude residue was purified by silica gel chromatography (0-75% EtOAc in hexanes) to give 19-1 as a clear solid, MS m/z (M+H) 411.1 found, 411.2 required.

### (±) cis-10-methoxy-3-[3-(4-methyl-1H-pyrrol-2-yl)-1,2,4-oxadiazol-5-yl]hexahydropyrido[1,2-b][2]benzazepin-6(2H)-one (19-2)

A solution of (±) N′-((cis-10-methoxy-6-oxo-1,2,3,4,6,11,12,12a-Octahydrobenzo[e]pyrido[1,2-a]azepine-3-carbonyl)oxy)-4-methyl-1H-pyrrole-2-carboximidamide 19-1 (0.012 g, 0.029 mmol) in DMA (0.146 mL) was irradiated at 130° C. for 40 minutes. The reaction contents were partitioned between EtOAc and water and the organic phase was washed with brine, dried over MgSO₄, filtered and concentrated. The crude residue was purified by silica gel chromatography (0-100% EtOAc in hexanes) to give 19-2 as a clear oil. MS m/z (M+H) 393.1922 found, 393.1921 required.

### Example 20

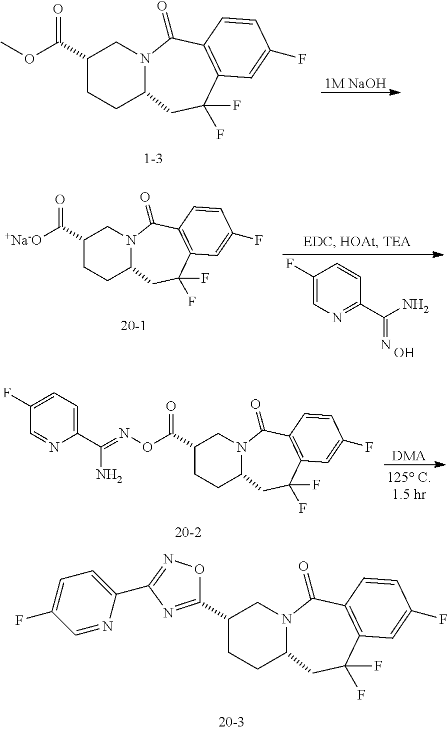

### Sodium-(±) cis-9,11,11-trifluoro-6-oxo-1,2,3,4,6,11,12,12a-octahydropyrido[1,2-b][2]benzazepine-3-Carboxylate (20-1)

A stirred solution of 1-3 (27.5 mg, 0.084 mmol) in THF (420 μL) was treated with 1N aq. NaOH (168 μL, 0.168 mmol). The reaction was stirred for 1 hour, then LCMS showed it had gone to completion. The reaction was condensed, and re-diluted with toluene, and solvent removed to give 20-1 as a white film MS m/z (M+H) 314.0 found, 314.1 required.

### 5-fluoro-N′-(±) cis-({[9,11,11-trifluoro-6-oxo-1,2,3,4,6,11,12,12a-octahydropyrido[1,2-b][2]benzazepin-3-yl]carbonyl}oxy)pyridine-2-carboximidamide (20-2)

A solution of 20-1 (5 mg, 0.015 mmol), EDC (5.72 mg, 0.030 mmol), HOAt (4.06 mg, 0.030 mmol) and TEA (10.39 pt, 0.075 mmol) in DMF (149 μL) was stirred at room temperature for 15 minutes. To this solution was added 5-fluoro-N′-hydroxypyridine-2-carboximidamide (2.78 mg, 0.018 mmol, prepared as described in WO 2002/068417), and the reaction was stirred overnight. The reaction was then diluted with EtOAc, and washed with H₂O several times. The organic phase was then dried over MgSO₄, filtered and condensed. Purification with silica gel column (4 g column 0-75% EtOAc in hexane) gave 20-2, MS m/z (M+H) 451.0 found, 451.1 required.

### (±) cis-9,11,11-trifluoro-3-[3-(5-fluoropuridin-2-yl)-1,2-4-oxadiazol-5-yl]-1,3,4,11,12,12a-hexahydropyrido[1,2-b][2]benzazepin-6(2H)-one (20-3)

A solution of 20-2 (15 mg, 0.033 mmol) in DMA (333 μL) was placed in a microwave vial and heated for 1.5 hr at 125° C. Upon completion of the reaction the solution was diluted with EtOAc and washed three times with water/brine. The organic layer was dried and condensed. Purification with silica gel column (4 g column 0-75% EtOAc in hexane) gave 20-3, MS m/z (M+H) 433.1282 found, 433.1209 required.

### Example 21

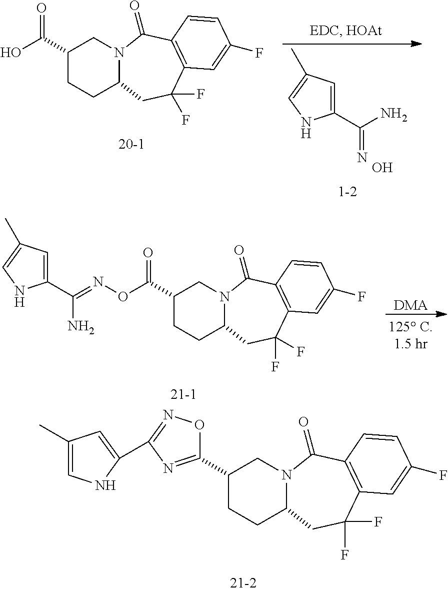

### (±)-4-methyl N′-({[cis-9,11,11-trifluoro-6-oxo-1,2,3,4,6,11,12,12a-octahydropyrido[1,2-b][2]benzazepin-3-yl]carbonyl}oxy)H-1pyrrole-2-carboximidamide (2H)

A solution of 20-1 (26 mg, 0.078 mmol), EDC (29.7 mg, 0.155 mmol), HOAt (21.1 mg, 0.155 mmol) and TEA (54 μL, 0.388 mmol) in DMF (776 μL) was stirred at room temperature for 15 minutes. To this solution was added the N′-hydroxy-4-methyl-1H-pyrrole-2-carboximidamide (1-2, 10.79 mg, 0.078 mmol, prepared as described in WO 2006/123257), and the reaction was stirred overnight. The reaction was then diluted with EtOAc, and washed with H₂O several times. The organic phase was then dried over MgSO₄, filtered and condensed. Purification with silica gel column (4 g column 0-75% EtOAc in hexane) gave 21-1, MS m/z (M+H) 435.0 found, 435.1 required.

### (±)cis-9,11,11-trifluoro-3-[3-(4-methyl-1H-pyrrol-2-yl)-1,2,4-oxadiazol-5-yl]-1,3,4,11,12,12a-hexahydropyrido[1,2-b][2]benzazepin-6(2H)-one (21-2)

A solution of 21-1 (21.6 mg, 0.050 mmol) in DMA (497 μL) was placed in a microwave vial and heated for 1.5 hr at 125° C. The reaction turned a dark brown color. The reaction was diluted with EtOAc and washed three times with water/brine. The organic layer was dried and condensed. Purification with silica gel column (4 g column 0-100% EtOAc in hexane) gave 21-2, MS m/z (M+H) 417.1533 found, 417.1460 required.

### Example 22

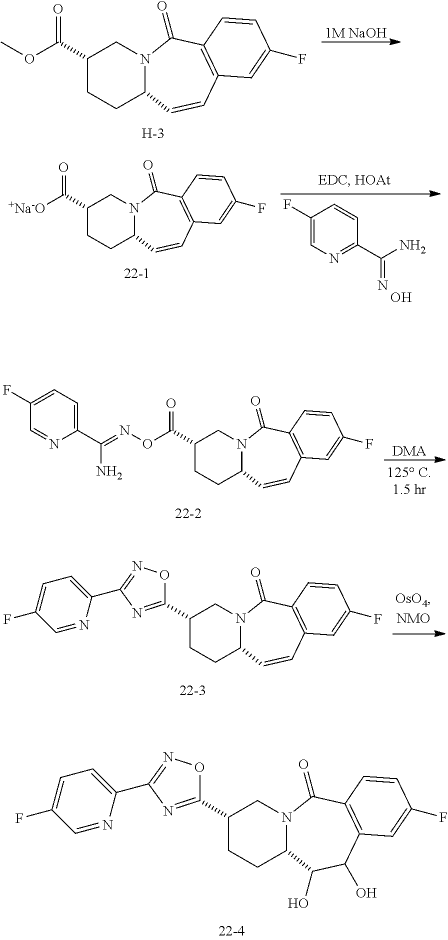

Sodium-(±) cis-9-fluoro-6-oxo-1,2,3,4,6,12a-hexahydropyrido[1,2-b][2]benzazepine-3-carboxylate (22-1)

A stirred solution of H-3 (5538 mg, 1.914 mmol) in THF (9.5 mL) was treated with 1N aq, NaOH (3.83 mL, 3.83 mmol). The reaction was stirred for 1 hour, and then LCMS showed it had gone to completion. The reaction was condensed, and re-diluted with toluene, and solvent removed to give 22-1 as a white film MS m/z (M+H) 276.0 found, 276.1 required.

### (±)cis-5-fluoro-N′-({[9,-fluoro-6-oxo-1,2,3,4,6,12a-hexahydropyrido[1,2-b][2]benzazepin-3-yl]carbonyl}oxy)pyridine-2-carboximidamide (22-2)

A solution of 22-1 (600 mg, 1.915 mmol), EDC (734 mg, 3.83 mmol), HOAt (521 mg, 3.83 mmol) and TEA (1.33 mL, 9.57 mmol) in DMF (19.9 mL) was stirred at room temperature for 15 minutes. To this solution was added the 5-fluoro-N′-hydroxypyridine-2-carboximidamide (356 mg, 2.298 mmol), and the reaction was stirred overnight. The reaction was then diluted with EtOAc, and washed with H₂O several times. The organic phase was then dried over MgSO₄, filtered and condensed. Purification with silica gel column (24 g column 0-75% EtOAc in hexane) gave 22-2, MS m/z (M+H) 413.3.0 found, 413.1 required

### (±) cis-9-fluoro-3-[3-(5-fluoropyridin-2-yl)-1,2-4-oxadiazol-5-yl}-1,3,4,12a-tetrahydropyrido[1,2-b][2]benzazepin-6(2H)-one (22-3)

A solution of 22-2 (456.4 mg, 1.107 mmol) in DMA (12 mL) was placed in a microwave vial and heated for 1.5 hr at 125° C. Upon completion of the reaction the solution was diluted with EtOAc and washed three times with water/brine. The organic layer was dried and condensed. Purification with silica gel column (24 g column 0-100% EtOAc in hexane) gave 14-3, MS m/z (M+H) 395.1308 found, 395.1241 required.

### (±) cis-9-fluoro-3-[3-(5-fluoropyridin-2-yl)-1,2-4-oxadiazol-5-yl}-11,12-dihydroxy-1,3,4,11,12,12a-hexahydropyrido[1,2-b][2]benzazepin-6(2H)-one (22-4)

To a solution of 22-3 (11.4 mg, 0.29 mmol) in acetone (126 μL) and H₂O (18.07 mL) was added the NMO (5.08 mg, 0.043 mmol) and 2.5% OsO₄ solution (7.26 μL, 0.578 μmol). This was stirred overnight, and then the reaction was worked up by the addition of saturated aqueous Na₂SO₃. The aqueous phase was extracted with EtOAc three times and the organic phase was dried over Na₂SO₄, filtered and condensed. Purification with silica gel column (4 g column 0-100% EtOAc in hexane) gave 22-4, MS m/z (M+H) 429.1371 found, 429.1296 required.

### Example 23

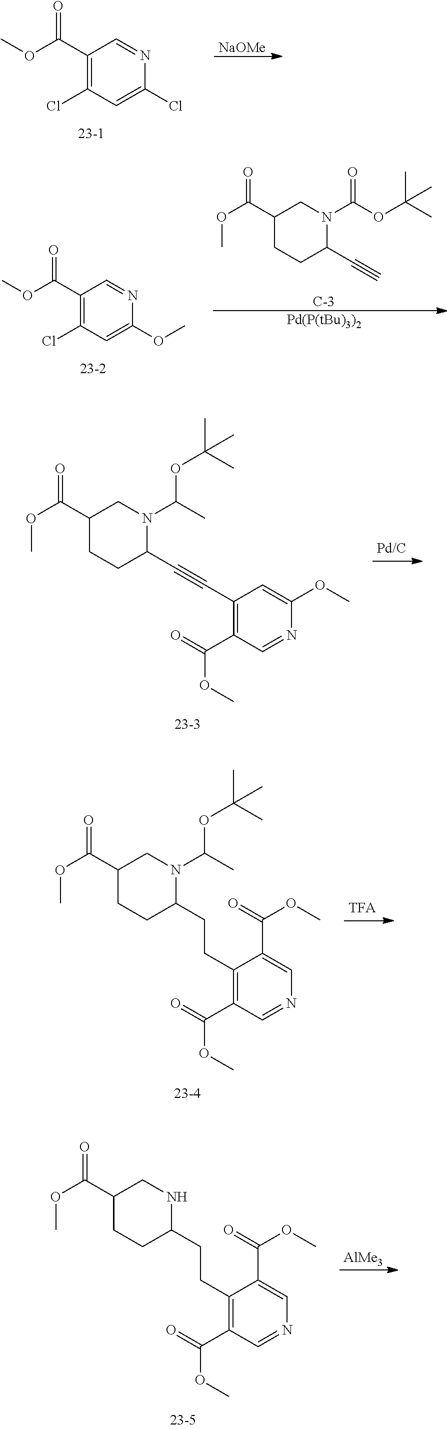

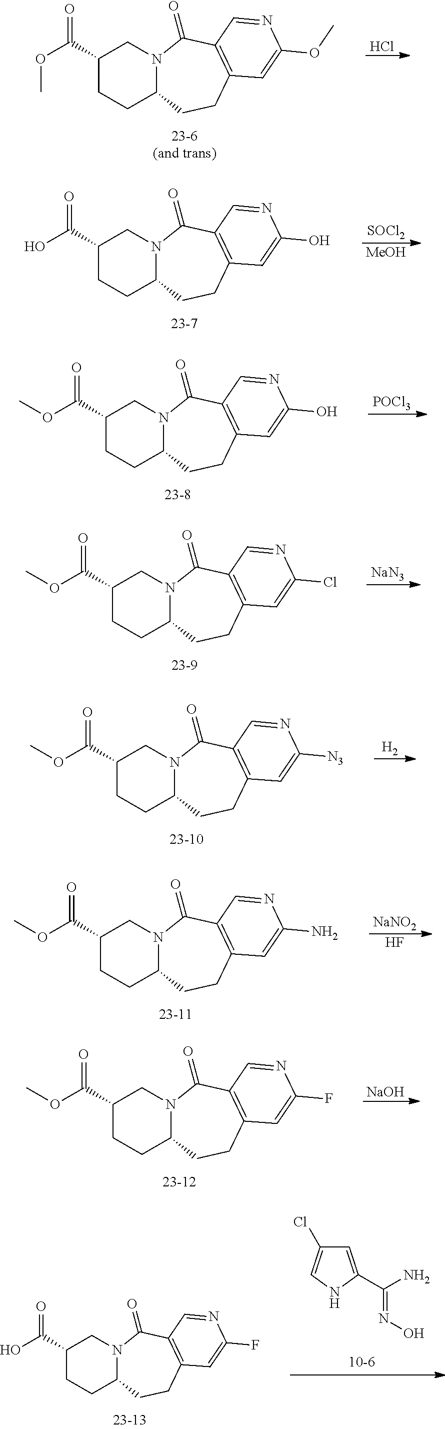

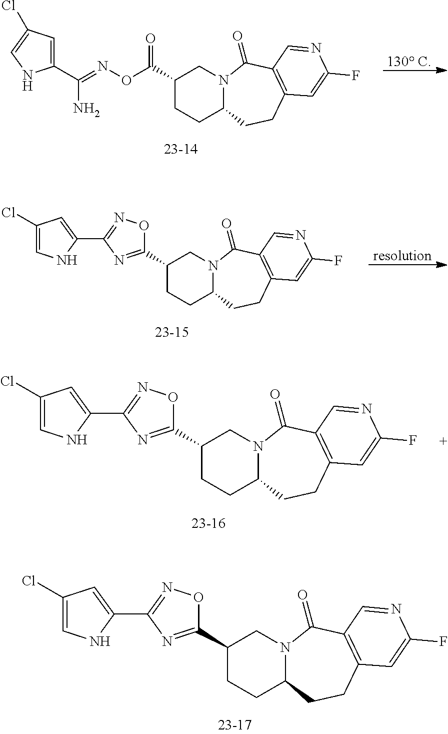

### Methyl 4-chloro-6-methoxypyridine-3-carboxylate (23-2)

To methyl 4,6-dichloropyridine-3-carboxylate (23-1) (1.00 g, 4.85 mmol) as a solution in DMSO (10 mL) cooled to 0° C. was added sodium methoxide in methanol (0.831 mL, 4.61 mmol) under N₂ and allowed to warm to RT. After 30 minutes, the reaction was diluted with water (1000 mL) and extracted with ethyl acetate (2×1000 mL), washed with water (2×1000 mL), dried (Na₂SO₄), filtered and the solvent was evaporated under reduced pressure. The resulting residue was purified by flash chromatography eluting with 0-25% ethyl acetate/hexanes to give the title compound (23-2) as a white solid. MS m/z (M+H) 202.1 found, 202.6 required.

### cis/trans-1-tert-butyl 3-methyl 6-{[2-methoxy-5-(methoxycarbonyl)pyridin-4-yl]ethynyl}piperidine-1,3-dicarboxylate (23-3)

To methyl 4-chloro-6-methoxypyridine-3-carboxylate (23-2) (6.796 g, 33.7 mmol) and cesium carbonate (21.97 g, 67.4 mmol) suspended in DMF (100 mL) in a flame-dried flask under N₂ was added cis/trans-1-tert-butyl 3-methyl 6-ethynylpiperidine-1,3-dicarboxylate (C-3) (10.45 g, 39.1 mmol) as a solution in DMF (30 mL). This was placed under vacuum and purged with N₂. Repeated twice. To this was added bis(tri-t-butylphosphine)palladium(0) (1.723 g, 3.37 mmol) and the reaction was purged once. The mixture was placed in an oil bath at 80° C. After 90 minutes, the reaction was allowed to cool to room temperature, water (500 mL) was added and the mixture was extracted with ethyl acetate (2×400 mL). The combined organic fractions were washed with dilute brine (2×500 mL), dried (Na₂SO₄), filtered and the solvent was evaporated under reduced pressure. The resulting residue (18 g) was purified by column chromatography eluting with 0-40% EtOAc/hexanes to afford the title compound (23-3). MS m/z (M+H) 433.2 found, 433.5 required.

### cis/trans-1-tert-butyl 3-methyl 6-{2-[2-methoxy-5-(methoxycarbonyl)pyridin-4-yl]ethyl}piperidine-1,3-dicarboxylate (23-4)

cis/trans-1-tert-Butyl 3-methyl 6-{[2-methoxy-5-(methoxycarbonyl)pyridin-4-yl]ethynyl}piperidine-1,3-dicarboxylate (24-3) (9.8 g, 22.66 mmol) was dissolved in methanol (100 mL) and evacuated and let up to N₂ twice. Pd/C (2.00 g, 18.8 mmol) was added to the solution and evacuated and let up to N₂. Evacuated and let up to H₂. After 24 hours, the reaction was evacuated and let up to N₂. Filtered through celite under N₂ and concentrated to afford the title compound (23-4). MS m/z (M+H) 437.2 found, 437.5 required.

### cis/trans-methyl 6-methoxy-4-{2-[5-(methoxycarbonyl)piperidin-2-yl]ethyl}pyridine-3-carboxylate (23-5)

To cis/trans-1-tert-butyl 3-methyl 6-{2-[2-methoxy-5-(methoxycarbonyl)pyridin-4-yl]ethyl}piperidine-1,3-dicarboxylate (23-4) (9.57 g, 21.92 mmol) in dichloromethane (20 mL) was added TFA (20 mL). After 1 hour, the reaction was concentrated to an oil, taken up in ethyl acetate and washed with saturated aqueous sodium carbonate and then water. The organic layer was dried (Na₂SO₄), filtered and concentrated. The resulting residue was purified by flash chromatography eluting with 0-5% methanol/dichloromethane to yield the title compound (23-5). MS m/z (M+H) 337.1 found, 337.4 required.

### cis-methyl 3-methoxy-12-oxo-5,6,6a,7,8,9,10,12-octahydrodipyrido[1,2-a:4′,3′-e]azepine-9-carboxylate (23-6)

To cis/trans-methyl 6-methoxy-4-{2-[5-(methoxycarbonyl)piperidin-2-yl]ethyl}pyridine-3-carboxylate (21-5) (5.206 g, 15.48 mmol) in DCM (40 mL) at −78° C. under N₂ was added trimethylaluminum (2M in toluene) (23.21 mL, 46.4 mmol). After 30 minutes, the reaction was allowed to warm to RT and stir under N₂. After 26 hours, the reaction was quenched with ice. The mixture was acidified with 1N HCl to pH1 (˜175 mL). This mixture was extracted with DCM. The combined organic fractions were dried (Na₂SO₄), filtered and concentrated to yield a mixture of cis and trans products. This was purified by flash chromatography eluting with 0-100% ethyl acetate/DCM to give the title compound (23-6). MS m/z (M+H) 305.1 found, 305.3 required.

### cis 3-hydroxy-12-oxo-5,6,6a,7,8,9,10,12-octahydrodipyrido[1,2-a:4′,3′-e]azepine-9-carboxylic acid (23-7)

cis-Methyl 3-methoxy-12-oxo-5,6,6a,7,8,9,10,12-octahydrodipyrido[1,2-a:4′,3′-e]azepine-9-carboxylate (23-6) (1.92 g, 6.31 mmol) dissolved in 6M hydrochloric acid (12 mL) was placed in an oil bath at 80° C. under a strong stream of N₂. After 16 h, the reaction had concentrated to yield only the title compound (23-7). MS m/z (M+H) 277.0 found, 277.3 required.

### cis-methyl 3-hydroxy-12-oxo-5,6,6a,7,8,9,10,12-octahydrodipyrido[1,2-a:4′,3′-e]azepine-9-carboxylate (23-8)

cis 3-Hydroxy-12-oxo-5,6,6a,7,8,9,10,12-octahydrodipyrido[1,2-a:4′,3′-e]azepine-9-carboxylic acid (21-7) (800 mg, 2.90 mmol) was suspended in thionyl chloride (10 mL) at RT and became a solution after stirring for 2 hours. The reaction was concentrated to a solid and suspended in ethyl acetate (100 mL), washed with saturated aqueous sodium bicarbonate (10 mL) and water (10 mL). Aqueous was back-extracted with ethyl acetate (2×100 mL). The extract was concentrated and purified by flash chromatography eluting with 0-5% methanol/DCM to afford the title compound (23-8). MS m/z (M+H) 291.1 found, 291.3 required.

### cis-methyl 3-chloro-12-oxo-5,6,6a,7,8,9,10,12-octahydrodipyrido[1,2-a:4′,3′-e]azepine-9-carboxylate (23-9)

To cis-methyl 3-hydroxy-12-oxo-5,6,6a,7,8,9,10,12-octahydrodipyrido[1,2-a:4′,3′-e]azepine-9-carboxylate (21-8) (0.277 g, 0.954 mmol) dissolved in a mixture of toluene (2 mL) and acetonitrile (1 mL) was added POCl₃ (0.439 g, 2.86 mmol) and placed in an oil bath at 100° C. under N₂. After 5 hours, the reaction was allowed to cool to RT and quenched with ice. The reaction was diluted with ethyl acetate (25 mL) and washed with saturated aqueous sodium carbonate (15 mL). The aqueous was extracted with ethyl acetate and the combined extracts were washed with water. The extract was dried (Na₂SO₄), filtered and concentrated. This was purified by flash chromatography eluting with 0-5% methanol/DCM to give the title compound (23-9). MS m/z (M+H) 309.0 found, 309.8 required.

### cis-methyl 3-azido-12-oxo-5,6,6a,7,8,9,10,12-octahydrodipyrido[1,2-a:4′,3′-e]azepine-9-carboxylate (23-10)

To cis-methyl 3-chloro-12-oxo-5,6,6a,7,8,9,10,12-octahydrodipyrido[1,2-a:4′,3′-e]azepine-9-carboxylate (23-9) (0.533 g, 1.726 mmol) suspended in DMSO (8 mL) was added sodium azide (0.168 g, 2.59 mmol) and the reaction was placed in an oil bath at 120° C. upon which the reaction became a solution. After 25 hours, the reaction was allowed to cool to RT. The reaction was diluted with water (50 mL) and extracted with ethyl acetate (2×50 mL). The combined extracts were washed with water (2×50 mL), dried (Na₂SO₄), filtered and concentrated to yield the title compound (23-10). MS m/z (M+H) 316.0 found, 316.3 required.

### cis-methyl 3-amino-12-oxo-5,6,6a,7,8,9,10,12-octahydrodipyrido[1,2-a:4′,3′-e]azepine-9-carboxylate (23-11)

cis-Methyl 3-azido-12-oxo-5,6,6a,7,8,9,10,12-octahydrodipyrido[1,2-a:4′,3′-e]azepine-9-carboxylate (21-10) (0.371 g, 1.177 mmol) was dissolved in methanol (10 mL) and EtOAc (10 mL). This was evacuated and purged with N₂ thrice. To this was added Pd/C (0.120 g, 1.128 mmol) and evacuated and purged as before. Evacuated and purged with H₂. After 18 hours, the reaction was evacuated and purged with N₂. Filtered under N₂ through celite and concentrated to afford the title compound (23-11). MS m/z (M+H) 290.1 found, 290.3 required.

### cis-methyl 3-fluoro-12-oxo-5,6,6a,7,8,9,10,12-octahydrodipyrido[1,2-a:4′,3′-e]azepine-9-carboxylate (23-12)

To cis-methyl 3-amino-12-oxo-5,6,6a,7,8,9,10,12-octahydrodipyrido[1,2-a:4′,3′-e]azepine-9-carboxylate (23-11) (0.289 g, 0.999 mmol) dissolved in 70% HF pyridine (3 mL) at 0° C. was added sodium nitrite (0.689 g, 9.99 mmol) slowly in portions. The reaction was allowed to warm to RT and after 10 minutes post addition, the reaction was quenched into a saturated aqueous sodium carbonate solution (30 mL) and sodium carbonate added until the pH was decidedly basic (˜12). The reaction was extracted with ethyl acetate (4×25 mL) and the combined extracts were dried (Na₂SO₄), filtered and concentrated to an oil. This was purified by flash chromatography eluting with 0-40% ethyl acetate/DCM to give the title compound (23-12). MS m/z (M+H) 293.0 found, 293.3 required.

### cis-3-fluoro-12-oxo-5,6,6a,7,8,9,10,12-octahydrodipyrido[1,2-a:4′,3′-e]azepine-9-carboxylic acid (23-13)

To cis-methyl 3-fluoro-12-oxo-5,6,6a,7,8,9,10,12-octahydrodipyrido[1,2-a:4′,3′-e]azepine-9-carboxylate (23-12) (0.128 g, 0.438 mmol) partially dissolved methanol (4 mL) was added 1N aqueous sodium hydroxide (0.876 mL, 0.876 mmol) at RT. The reaction became a solution. After 1.5 hours, the reaction was quenched with HCl (0.876 ml, 0.876 mmol) and extracted with ethyl acetate. Extract dried (Na₂SO₄), filtered and concentrated to yield the title compound (23-13). MS m/z (M+H) 279.0 found, 279.3 required.

### cis-4-chloro-N′-{[(3-fluoro-12-oxo-5,6,6a,7,8,9,10,12-octahydrodipyrido[1,2-a:4′,3′-e]azepin-9-yl)carbonyl]oxy}-1H-pyrrole-2-carboximidamide (23-14)

cis-3-Fluoro-12-oxo-5,6,6a,7,8,9,10,12-octahydrodipyrido[1,2-a:4′,3′-e]azepine-9-carboxylic acid (23-13) (0.120 g, 0.431 mmol) was suspended in DCM (4 mL) and thionyl chloride (0.157 mL, 2.156 mmol) was added. The reaction eventually resolved into a solution. After 1 hour, the reaction was diluted with toluene (2 mL) and concentrated to an oil. This oil, as a solution in DCM (2 mL), was added to a suspension of 4-chloro-N′-hydroxy-1H-pyrrole-2-carboximidamide 10-6 (76 mg, 0.475 mmol) and triethylamine (0.120 mL, 0.863 mmol) in DCM:THF (1:1, 2 mL). After 1 hour, the reaction was filtered to afford the title compound (23-14). MS m/z (M+H) 420.0 found, 420.8 required.

### cis-9-[3-(4-chloro-1H-pyrrol-2-yl)-1,2,4-oxadiazol-5-yl]-3-fluoro-6,6a,7,8,9,10-hexahydrodipyrido[1,2-a:4′,3′-e]azepin-12(5H)-one (23-15)

cis-4-Chloro-N′-{[(3-fluoro-12-oxo-5,6,6a,7,8,9,10,12-octahydrodipyrido[1,2-a:4′,3′-e]azepin-9-yl)carbonyl]oxy}-1H-pyrrole-2-carboximidamide (23-14) (0.109 g, 0.260 mmol) dissolved in DMA (2 mL) was placed in a Biotage microwave at 130° C. for 20 minutes. Upon cooling to RT, the reaction was diluted with water (10 mL) and extracted with ethyl acetate (2×25 mL). The extracts were combined and washed with water (2×25 mL), dried (Na₂SO₄), filtered and the solvent was evaporated under reduced pressure. The resulting residue was purified by flash chromatography eluting with 0-25% ethyl acetate/DCM to afford the title compound as a racemate (23-15). MS m/z (M+H) 402.0 found, 402.8 required.

### (6aS,9S)-9-[3-(4-chloro-1H-pyrrol-2-yl)-1,2,4-oxadiazol-5-yl]-3-fluoro-6,6a,7,8,9,10-hexahydrodipyrido[1,2-a:4′,3′-e]azepin-12(5H)-one (23-16)

Racemic cis-9-[3-(4-chloro-1H-pyrrol-2-yl)-1,2,4-oxadiazol-5-yl]-3-fluoro-6,6a,7,8,9,10-hexahydrodipyrido[1,2-a:4′,3′-e]azepin-12(5H)-one (23-15) (0.060 g, 0.149 mmol) was enantiomericly separated utilizing a Chiralpak IC column eluting under super-critical fluid conditions with 45% methanol and carbon dioxide. The first eluting enantiomer yielded the title compound (23-16). MS m/z (M+H) 402.1136 found, 402.1128 required.

### (6aR,9R)-9-[3-(4-chloro-1H-pyrrol-2-yl)-1,2,4-oxadiazol-5-yl]-3-fluoro-6,6a,7,8,9,10-hexahydrodipyrido[1,2-a:4′,3′-e]azepin-12(5H)-one (23-17)

Racemic cis-9-[3-(4-chloro-1H-pyrrol-2-yl)-1,2,4-oxadiazol-5-yl]-3-fluoro-6,6a,7,8,9,10-hexahydrodipyrido[1,2-a:4′,3′-e]azepin-12(5H)-one (23-15) (0.060 g, 0.149 mmol) was enantiomericly separated utilizing a Chiralpak IC column eluting under super-critical fluid conditions with 45% methanol and carbon dioxide. The second eluting enantiomer yielded the title compound (23-17). MS m/z (M+H) 402.1134 found, 402.1128 required.

### Example 24

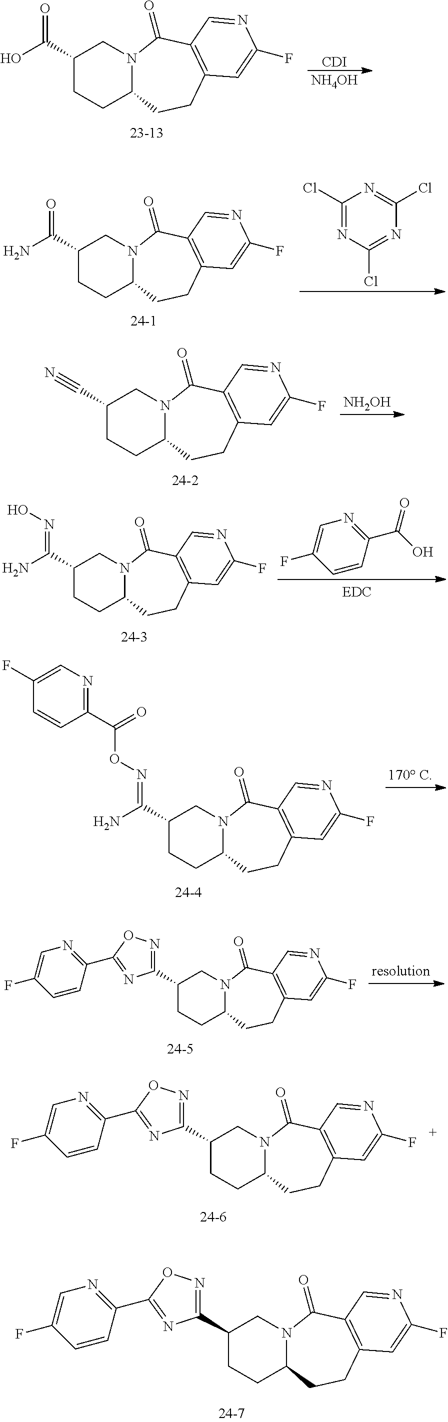

### cis-3-fluoro-12-oxo-5,6,6a,7,8,9,10,12-octahydrodipyrido[1,2-a:4′,3′-e]azepine-9-carboxamide (24-1)

To a suspension of cis-3-fluoro-12-oxo-5,6,6a,7,8,9,10,12-octahydrodipyrido[1,2-a:4′,3′-e]azepine-9-carboxylic acid (23-13) (0.650 g, 2.336 mmol) in ACN (10 mL) was added carbonyl diimidazole (0.568 g, 3.50 mmol). After 90 minutes a solution had formed and 29% aqueous ammonium hydroxide (0.174 mL, 2.57 mmol) was added. After 4 hours, the reaction was diluted with DCM (150 mL) and washed with water (25 mL). The organic was dried (Na₂SO₄), filtered and concentrated to yield the title compound (24-1). MS m/z (M+H) 278.1 found, 278.3 required.

### cis-3-fluoro-12-oxo-5,6,6a,7,8,9,10,12-octahydrodipyrido[1,2-a:4′,3′-e]azepine-9-carbonitrile (24-2)

To cis-3-fluoro-12-oxo-5,6,6a,7,8,9,10,12-octahydrodipyrido[1,2-a:4′,3′-e]azepine-9-carboxamide (24-1) (0.648 g, 2.337 mmol) in DMF (10 mL) was added 2,4,6-trichloro-1,3,5-triazine (0.345 g, 1.869 mmol) as a solid at RT. After 16 hours, the reaction was cooled to 0° C. and water was added (100 mL). This was extracted with ethyl acetate (2×100 mL) and the combined extracts were washed with water (2×100 mL), dried (Na₂SO₄), filtered and concentrated. The resulting residue was purified by flash chromatography eluting with 0-25% ethyl acetate/DCM to yield the title compound (24-2). MS m/z (M+H) 260.1 found, 260.3 required.

### cis-3-fluoro-N′-hydroxy-12-oxo-5,6,6a,7,8,9,10,12-octahydrodipyrido[1,2-a:4′,3′-e]azepine-9-carboximidamide (24-3)

cis-3-Fluoro-12-oxo-5,6,6a,7,8,9,10,12-octahydrodipyrido[1,2-a:4′,3′-e]azepine-9-carbonitrile (24-2) (0.312 g, 1.203 mmol) was suspended in absolute ethanol (10 mL) and 50% aqueous hydroxylamine (0.092 mL, 1.504 mmol) was added. This was placed under N₂ and then in an oil bath maintained at 45° C. After 48 hours, the reaction was allowed to cool to RT and concentrated to afford the title compound (24-3). MS m/z (M+H) 293.1 found, 293.3 required.

### cis-3-fluoro-N′-{[(5-fluoropyridin-2-yl)carbonyl]oxy}-12-oxo-5,6,6a,7,8,9,10,12-octahydrodipyrido[1,2-a:4′,3′-e]azepine-9-carboximidamide (24-4)

5-Fluoropyridine-2-carboxylic acid (0.036 g, 0.257 mmol), EDC (0.049 mg, 0.257 mmol) and 3H-[1,2,3]triazolo[4,5-b]pyridin-3-ol hydrate (0.039 mg, 0.257 mmol) were dissolved in DMF (2 mL). After 5 minutes of stirring, cis-3-fluoro-N′-hydroxy-12-oxo-5,6,6a,7,8,9,10,12-octahydrodipyrido[1,2-a:4′,3′-e]azepine-9-carboximidamide (24-3) (0.075 g, 0.257 mmol) was added as a solid. After 10 minutes, the reaction was diluted with water and extracted with ethyl acetate. Extracts were combined and washed with water, dried, filtered and concentrated to afford the title compound (24-4). MS m/z (M+H) 416.0 found, 416.4 required.

### cis-3-fluoro-9-[5-(5-fluoropyridin-2-yl)-1,2,4-oxadiazol-3-yl]-6,6a,7,8,9,10-hexahydrodipyrido[1,2-a:4′,3′-e]azepin-12(5H)-one (22-5)

cis-3-Fluoro-N′-{[(5-fluoropyridin-2-yl)carbonyl]oxy}-12-oxo-5,6,6a,7,8,9,10,12-octahydrodipyrido[1,2-a:4′,3′-e]azepine-9-carboximidamide (24-4) (0.090 g, 0.217 mmol) dissolved in DMA (2 mL) was heated in a Biotage microwave reactor at 170° C. for 30 minutes after which time the mixture was allowed to cool to RT. The reaction was diluted with water (20 mL) and extracted with ethyl acetate (3×25 mL) and the combined extracts were washed with water (2×25 mL), dried (Na₂SO₄), filtered and concentrated. The resulting residue was purified by flash chromatography eluting with 0-60% ethyl acetate/hexanes to give the title compound (24-5).

### (6aS,9S)-3-fluoro-9-[5-(5-fluoropyridin-2-yl)-1,2,4-oxadiazol-3-yl]-6,6a,7,8,9,10-hexahydrodipyrido[1,2-a:4′,3′-e]azepin-12(5H)-one (22-6)

Racemic cis-3-fluoro-9-[5-(5-fluoropyridin-2-yl)-1,2,4-oxadiazol-3-yl]-6,6a,7,8,9,10-hexahydrodipyrido[1,2-a:4′,3′-e]azepin-12(5H)-one (22-5) (0.050 g, 0.126 mmol) was enantiomericly separated utilizing a Chiralpak AD-H column eluting under super-critical fluid conditions with 40% IPA and carbon dioxide. The second eluting enantiomer yielded the title compound (24-6). MS m/z (M+H) 398.1427 found, 398.1423 required.

### (6aR,9R)-3-fluoro-9-[5-(5-fluoropyridin-2-yl)-1,2,4-oxadiazol-3-yl]-6,6a,7,8,9,10-hexahydrodipyrido[1,2-a:4′,3′-e]azepin-12(5H)-one (24-7)

Racemic cis-3-fluoro-9-[5-(5-fluoropyridin-2-yl)-1,2,4-oxadiazol-3-yl]-6,6a,7,8,9,10-hexahydrodipyrido[1,2-a:4′,3′-e]azepin-12(5H)-one (24-5) (0.050 g, 0.126 mmol) was enantiomericly separated utilizing a Chiralpak AD-H column eluting under super-critical fluid conditions with 40% IPA and carbon dioxide. The first eluting enantiomer yielded the title compound (22-7). MS m/z (M+H) 398.1425 found, 398.1423 required.

### Example 25

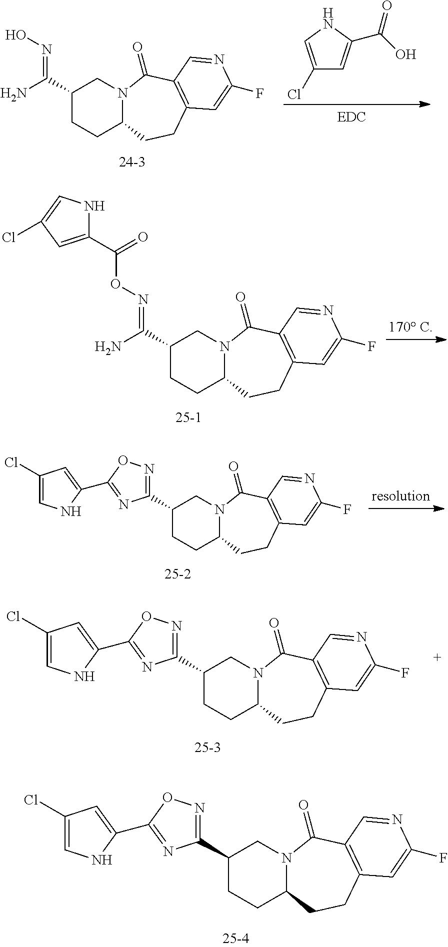

### cis-N′-{[(4-chloro-1H-pyrrol-2-yl)carbonyl]oxy}-3-fluoro-12-oxo-5,6,6a,7,8,9,10,12-octahydrodipyrido[1,2-a:4′,3′-e]azepine-9-carboximidamide (25-1)

4-Chloro-1H-pyrrole-2-carboxylic acid (0.087 g, 0.599 mmol), EDC (0.115 g, 0.599 mmol) and 3H-[1,2,3]triazolo[4,5-b]pyridin-3-ol hydrate (0.092 g, 0.599 mmol) were dissolved in DMF (2 mL) and stirred for 5 minutes. After this time, cis-3-fluoro-N′-hydroxy-12-oxo-5,6,6a,7,8,9,10,12-octahydrodipyrido[1,2-a:4′,3′-e]azepine-9-carboximidamide (24-3) (0.175 g, 0.599 mmol) was added as a solid. After 30 minutes, the reaction was diluted with water and extracted with ethyl acetate. The combined extracts were washed with water, dried (Na₂SO₄), filtered and concentrated to afford the title compound (25-1). MS m/z (M+H) 420.0 found, 420.8 required.

### 9-[5-(4-chloro-1H-pyrrol-2-yl)-1,2,4-oxadiazol-3-yl]-3-fluoro-6,6a,7,8,9,10-hexahydrodipyrido[1,2-a:4′,3′-e]azepin-12(5H)-one (25-2)

cis-N′-{[(4-Chloro-1H-pyrrol-2-yl)carbonyl]oxy}-3-fluoro-12-oxo-5,6,6a,7,8,9,10,12-octahydrodipyrido[1,2-a:4′,3′-e]azepine-9-carboximidamide (25-1) (0.251 g, 0.598 mmol) dissolved in DMA (2 mL) was heated in a Biotage microwave reactor at 170° C. for 60 minutes. After this time, the reaction was allowed to cool to RT and was diluted with water, extracted with ethyl acetate. The combined organic extracts were washed with water, dried (Na₂SO₄), filtered and concentrated. The resulting residue was purified by flash chromatography eluting with 0-60% ethyl acetate/hexanes to yield the title compound (25-2). MS m/z (M+H) 402.0 found, 402.8 required.

### (6aS,9S)-9-[5-(4-chloro-1H-pyrrol-2-yl)-1,2,4-oxadiazol-3-yl]-3-fluoro-6,6a,7,89,10-hexahydrodipyridol[1,2-a:4′,3′-e]azepin-12(5H)-one (25-3)

Racemic 9-[5-(4-chloro-1H-pyrrol-2-yl)-1,2,4-oxadiazol-3-yl]-3-fluoro-6,6a,7,8,9,10-hexahydrodipyrido[1,2-a:4′,3′-e]azepin-12(5H)-one (25-2) (0.125 g, 0.311 mmol) was enantiomericly separated utilizing a Chiralpak AD column eluting with 60% IPA and hexanes. The second eluting enantiomer yielded the title compound (25-3). MS m/z (M+H) 402.1133 found, 402.1128 required.

### (6aR,9R)-9-[5-(4-chloro-1H-pyrrol-2-yl)-1,2,4-oxadiazol-3-yl]-3-fluoro-6,6a,7,8,9,10-hexahydrodipyrido[1,2-a:4′,3′-e]azepin-12(5H)-one (25-4)

Racemic 9-[5-(4-chloro-1H-pyrrol-2-yl)-1,2,4-oxadiazol-3-yl]-3-fluoro-6,6a,7,8,9,10-hexahydrodipyrido[1,2-a:4′,3′-e]azepin-12(5H)-one (25-2) (0.125 g, 0.311 mmol) was enantiomericly separated utilizing a Chiralpak AD column eluting with 60% IPA and hexanes. The first eluting enantiomer yielded the title compound (25-4). MS m/z (M+H) 402.1132 found, 402.1128 required.

### Example 26

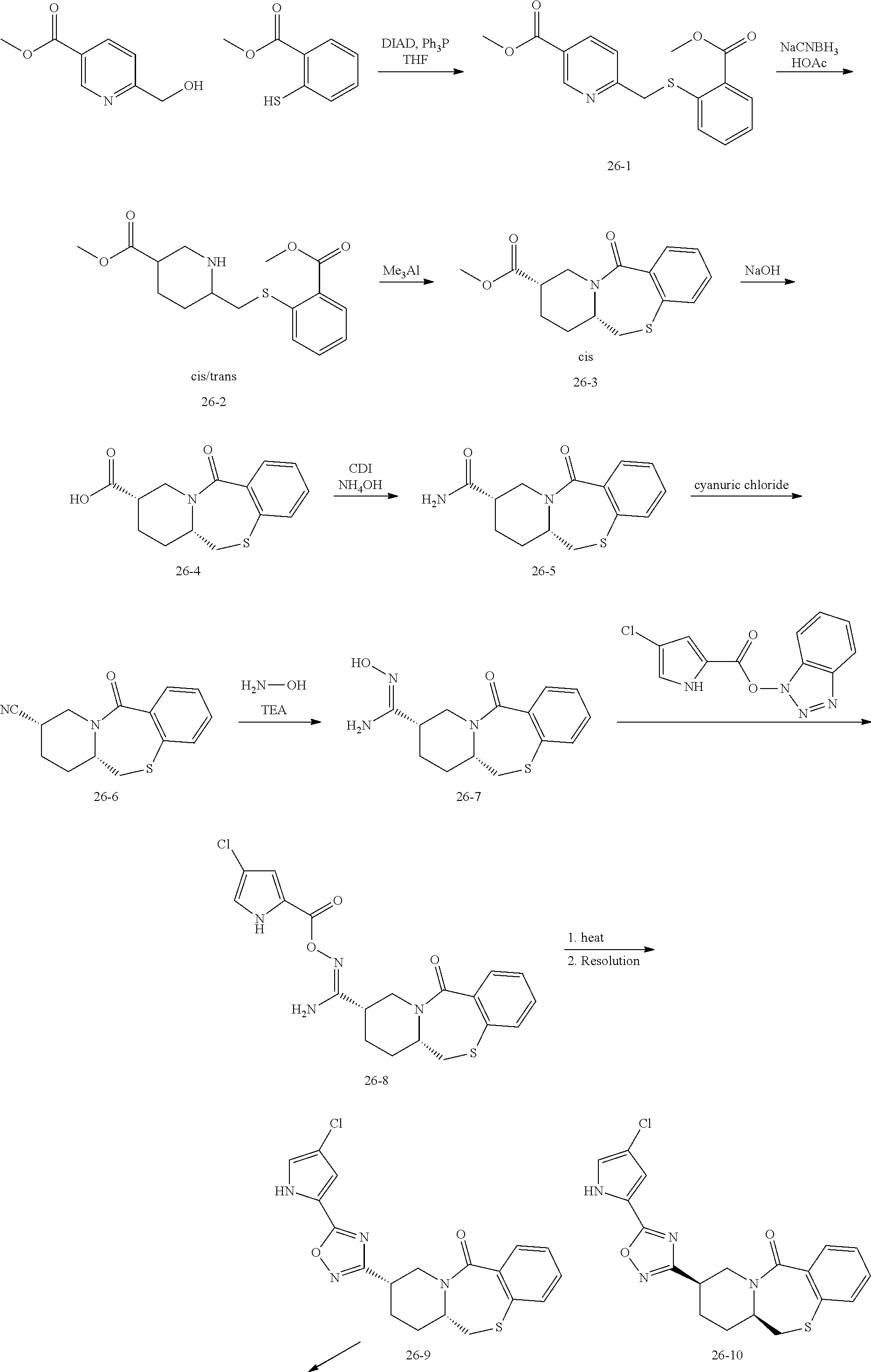

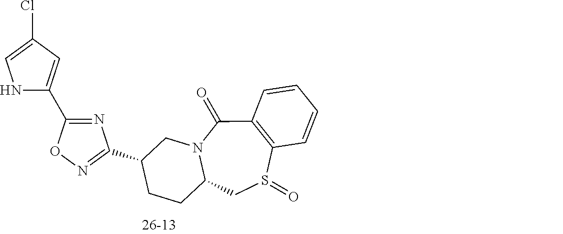

### methyl 6-({[2-(methoxycarbonyl)phenyl]sulfanyl}methyl)pyridine-3-carboxylate (26-1)

A solution of methyl 6-(hydroxymethyl)pyridine-3-carboxylate (2.00 g, 11.96 mmol) in THF (50 ml) was treated with methyl thiosalicyclate (2.01 g, 11.96 mmol) and triphenylphosphine (3.45 g, 13.16 mmol) and the resulting solution was cooled over an ice bath for 5 minutes. To the reaction mixture was added diisopropylazodicarboxylate (2.675 mL, 13.76 mmol) and the resulting mixture stirred cold for 30 minutes and allowed to warmed to 25° C. and stirred for an additional 60 minutes. After this time, the mixture was partitioned between CH₂Cl₂ and water. The organic extract was dried over MgSO₄, filtered and concentrated in vacuo. This residue was purified by column chromatography using a biotage column (65M) and eluting with a gradient of 0-25% EtOAc/hexane. The desired fractions were combined and concentrated in vacuo to provide 26-1. m/z (M+H) 318.0 found, 318.1 required.

### cis/trans-methyl 6-({[2-(methoxycarbonyl)phenyl]sulfanyl}methyl)piperidine-3-carboxylate (26-2)

A solution of 26-1 (4.06 g, 10.11 mmol) was dissolved in acetic acid (100 mL) and cooled over ice bath. This solution was treated with NaCNBH₃ (3.81 g, 60.6 mmol) portionwise over 15 minutes. The cooling bath was removed and the mixture was stirred for 5 hours at 25° C. The mixture was diluted with EtOAc (600 mL) and quenched with crushed ice (100 g). This mixture was treated with Na₂CO₃ to basic pH and the resulting layers were partitioned and the aqueous was further extracted with EtOAc (3×). The combined organic extracts were washed with brine, dried over MgSO₄, filtered and evaporated in vacuo. This residue was purified by column chromatography using a silica gel chromatography eluting with a gradient of 0-20% aq NH₄OH/CH₃CN. The desired fractions were combined to yield 26-2. m/z (M+H) 324.1 found, 324.1 required.

### methyl (6aS,9S)-12-oxo-6,6a,7,8,9,10-hexahydro-12H-pyrido[2,1-c][1,4]benzothiazepine-9-carboxylate (26-3)

A solution of 26-2 (820 mg, 2.54 mmol) in CH₂Cl₂ (25 mL) was cooled over a dry ice/acetone bath and treated with 2M Me₃Al (2.53 ml, 5.66 mmol) and then stirred at 25° C. for 16 hours. After this time, the reaction was cooled to 0° C. and quenched with crushed ice (50 g) until no bubbles formed. Added 25 mL of 1N HCl and let stir until mixture is clear. The resulting mixture was partitioned and the aqueous layer back extracted with CH₂Cl₂ (3×). The combined extracts were washed with brine (1×), dried over MgSO₄, filtered and the solvent evaporated in vacuo. This residue was purified by column chromatography using silica gel chromatography (0-100% EtOAc/hex) and collected peak 1 to yield 26-3. ¹H NMR (400 MHz, CDCl₃): δ 7.56 (dd, J=7.4, 1.7 Hz, 1H); 7.44-7.31 (m, 3H); 4.93 (dd, J=13.8, 4.7 Hz, 1H); 3.76 (m, 1H); 3.70 (s, 3H); 3.25 (t, J=12.4 Hz, 1H); 3.01-2.92 (m, 2H); 2.53 (tt, J=11.9, 4.2 Hz, 1H); 2.03-1.96 (m, 1H); 1.91-1.79 (m, 1H); 1.74-1.65 (m, 2H). m/z (M+H) 292.1 found, 292.1 required.

### 12-oxo-6,6a,7,8,9,10-hexahydro-12H-pyrido[2,1-c][1,4]benzothiazepine-9-carboxylic acid (26-4)

To a solution of 26-3 (1 g, 3.43 mmol) in THF/EtOH (10 ml:10 ml) was added 1N NaOH (10.30 ml, 10.30 mmol) and the resulting mixture stirred overnight. The organic solvents were removed and the aqueous layer was acidified to pH4-5 using 1N HCl. The aqueous layer was washed 3× with EtOAc and the combined organic layers were washed with brine (1×), dried over MgSO₄, filtered, and concentrated to yield 775 mg of the title compound 26-4. m/z (M+H) 278.0 found, 278.1 required.

### 12-oxo-6,6a,7,8,9,10-hexahydro-12H-pyrido[2,1-c][1,4]benzothiazepine-9-carboxamide (26-5)

To a suspension of 26-4 (775 mg, 2.79 mmol) in acetonitrile (30 ml) was added CDI (680 mg, 4.19 mmol) and the resulting mixture stirred for 15 min, Ammonium hydroxide (2813 μl, 41.9 mmol) was added and the reaction stirred overnight. The mixture was diluted with water and extract with EtOAc (3×). The combined organic layers were washed with 10% citric acid (2×), brine (1×), dried over MgSO₄, filtered, and concentrated to dryness. The crude residue was passed through small silica plug to afford 620 mg of the title compound 26-5. m/z (M+H) 277.0 found, 277.1 required.

### 12-oxo-6,6a,7,8,9,10-hexahydro-12H-pyrido[2,1-c][1,4]benzothiazepine-9-carbonitrile (26-6)

To a solution of 26-5 (620 mg, 2.24 mmol) in DMF (22 ml) at 0° C. was added cyanuric chloride (496 mg, 2.69 mmol). The ice bath was removed and the reaction warmed to rt over an hour. Diluted the reaction mixture with water and extracted the aqueous layer 2× with EtOAc. The combined organic layers were washed with water (3×), sodium bicarbonate (2×), brine 2×, dried over MgSO₄, and concentrated. The crude residue was purified using silica gel chromatography (10-100% EtOAc/hex) to afford 868 mg of 26-6. m/z (M+H) 259.0 found, 259.0 required.

### N′-hydroxy-12-oxo-6,6a,7,8,9,10-hexahydro-12H-pyrido[2,1-e][1,4]benzothiazepine-9-carboximidamide (26-7)

To a solution of 26-6 (500 mg, 1.935 mmol) in EtOH (1.94E+04 μl) was added TEA (313 mg, 3.10 mmol) and hydroxylamine hydrochloride (161 mg, 2.323 mmol). The mixture was heated in a sealed tube overnight at 85° C. The crude reaction mixture was concentrated and used with no further purification (26-7). m/z (M+H) 292.0 found, 292.1 required.

### N′-{[(4-chloro-1H-pyrrol-2-yl)carbonyl]oxy}-12-oxo-6,6a,7,8,9,10-hexahydro-12H-pyrido[2,1-c][1,4]benzothiazepine-9-carboximidamide (26-8)

To a solution of 26-7 (519 mg, 1.781 mmol) in DCM (17 mL) was added TEA (751 μl, 5.34 mmol), 1-{[(4-chloro-1H-pyrrol-2-yl)carbonyl]oxy}-1H-benzotriazole (655 mg, 2.494 mmol) and the resulting mixture stirred overnight. The crude reaction was diluted with water and extracted with DCM (2×). The combined organic extracts were washed with water (2×), brine (1×), dried over MgSO₄, and concentrated. The crude residue was used with no further purification (26-8). m/z (M+H) 418.9 found, 419.0 required.

### (6aS,9S)-9-[5-(4-chloro-1H-pyrrol-2-yl)-1,2,4-oxadiazol-3-yl]-6,6a,7,8,9,10-hexahydro-12H-pyrido[2,1-c][1,4]benzothiazepin-12-one (12-9) and (6aR,9R)-9-[5-(4-chloro-1H-pyrrol-2-yl)-1,2,4-oxadiazol-3-yl]-6,6a,7,8,9,10-hexahydro-12H-pyrido[2,1-c][1,4]benzothiazepin-12-one (26-10)

To solution of 26-8 (746 mg, 1.781 mmol) in DMA (10 mL) was heated in the microwave for a total of 70 min (20 min, 20 min, and 30 min) in the microwave at 120° C. Diluted with water and extracted with EtOAc (3×). The combined organic layers were washed with water (3×), brine (2×), dried over MgSO₄, and concentrated. The crude residue was purified using silica gel chromatography (10-100% EtOAc/hex) to afford 232 mg of the racemic product. Chiral separation using ChiralPak IC SFC (30×250 mm, 5μ) 50:50 CO₂:EtOH, 70 mL/min afforded 26-9 and 26-10. ¹H NMR (400 MHz, CDCl₃): δ 9.77 (s, 1H); 7.62-7.57 (m, 1H); 7.47-7.33 (m, 3H); 6.92 (s, 1H); 5.04 (dd, J=13.4, 4.0 Hz, 1H); 3.88-3.81 (m, 1H); 3.34 (t, J=12.5 Hz, 1H); 3.13 (t, J=12.9 Hz, 1H); 3.05-2.97 (m, 2H); 2.10 (m, 1H); 1.78 (m, 1H); 1.22 (m, 3H). m/z (M+H) 401.0837 found, 401.0834 required.

### (6aS,9S)-9-[5-(4-chloro-1H-pyrrol-2-yl)-1,2,4-oxadiazol-3-yl]-6,6a,7,8,9,10-hexahydro-12H-pyrido[2,1-c][1,4]benzothiazepin-12-one 5-oxide (26-13)

A solution of 26-9 (60 mg, 0.150 mmol) in CH₂Cl₂ (1.4 mL) was added MCPBA (34.4 mg, 0.150 mmol) and the resulting mixture stirred overnight. The reaction mixture was concentrated to dryness and purified using silica gel chromatography (50-100% EtoAc/hex) to yield 12 mg of 26-13. ¹H NMR (400 MHz, DMSO): δ 12.72 (s, 1H); 7.71-7.66 (m, 2H), 7.65-7.61 (m, 2H); 7.27 (s, 1H); 7.01 (s, 1H); 4.74 (m, 1H); 4.00 (m, 1H); 3.73-3.66 (m, 1H); 3.12-3.00 (m, 3H); 2.04 (m, 1H); 1.87 (m, 2H); 1.67 (m, 1H). m/z (M+H) 417.0800 found, 417.0783 required.

### Example 27

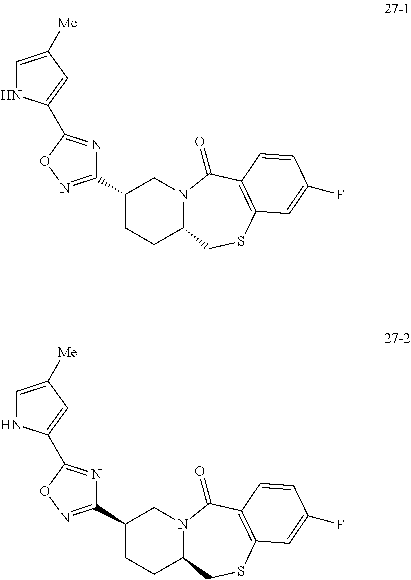 [SMILES: Cc1c[nH]c(-c2nc([C@H]3CC[C@H]4CSc5cc(F)ccc5C(=O)N4C3)no2)c1 ; Cc1c[nH]c(-c2nc([C@@H]3CC[C@@H]4CSc5cc(F)ccc5C(=O)N4C3)no2)c1]

### (6aS,9S)-9-[5-(4-methyl-1H-pyrrol-2-yl)-1,2,4-oxadiazol-3-yl]-6,6a,7,8,9,10-hexahydro-12H-pyrido[2,1-c][1,4]benzothiazepin-12-one (27-1) and (6aR,9R)-9-[5-(4-methyl-1H-pyrrol-2-yl)-1,2,4-oxadiazol-3-yl]-6,6a,7,8,9,10-hexahydro-12H-pyrido[2,1-c][1,4]benzothiazepin-12-one (27-2)

The title compounds were prepared in a manner identical to that described in Example 26-9 and 26-10 starting with intermediate 26-7 and using 1-{[(4-methyl-1H-pyrrol-2-yl)carbonyl]oxy}-1H-benzotriazole in place of 1-{[(4-chloro-1H-pyrrol-2-yl)carbonyl]oxy}-1H-benzotriazole. ¹H NMR (400 MHz, CDCl₃): δ 9.18 (s, 1H); 7.63-7.57 (m, 1H); 7.47-7.34 (m, 3H); 6.88 (s, 1H); 6.82 (s, 1H); 5.04 (dd, J=13.5, 4.1 Hz, 1H); 3.90-3.82 (m, 1H); 3.41-3.31 (m, 1H); 3.22-3.12 (m, 1H); 3.08-2.99 (m, 2H); 2.13 (s, 3H); 2.03-1.95 (m, 1H); 1.92-1.76 (m, 3H). m/z (M+H) 381.1383 found, 381.1380 required.

### Example 28

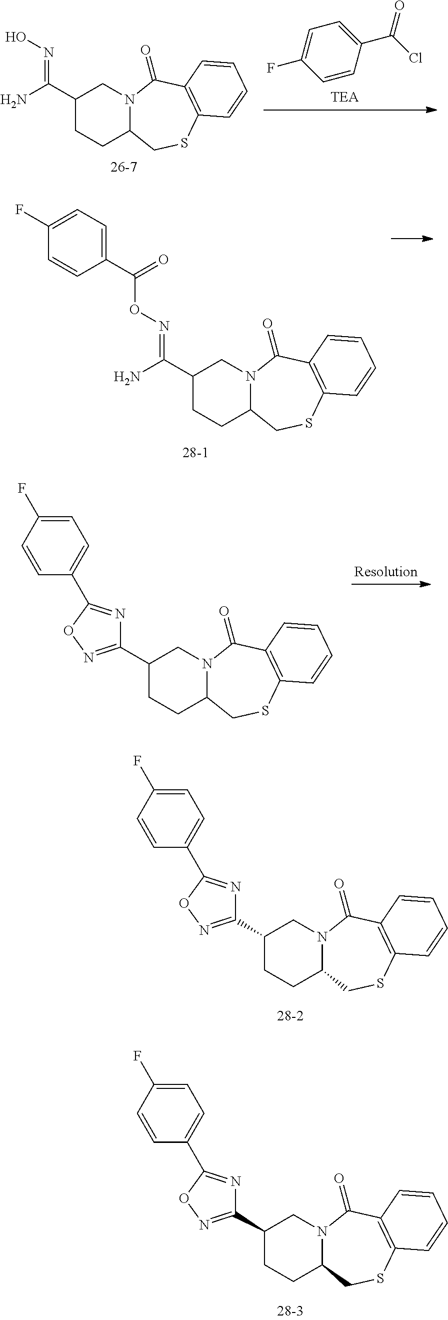

### N′-{[(4-fluorophenyl)carbonyl]oxy}-12-oxo-6,6a,7,8,9,10-hexahydro-12H-pyrido[2,1-c][1,4]benzothiazepine-9-carboximidamide (28-1)

In a manner essentially identical to that described in Example 26-8 above for the synthesis of 28-1, 4-fluorobenzoyl chloride was used in place of 1-{[(4-chloro-1H-pyrrol-2-yl)carbonyl]oxy}-1H-benzotriazole. m/z (M+H) 414.1 found, 414.1 required.

### (6aS,9S)-9-[5-(4-fluorophenyl)-1,2,4-oxadiazol-3-yl]-6,6a,7,8,9,10-hexahydro-12H-pyrido[2,1-c][1,4]benzothiazepin-12-one (28-2) and (6aR,9R)-9-[5-(4-fluorophenyl)-1,2,4-oxadiazol-3-yl]-6,6a,7,8,9,10-hexahydro-12H-pyrido[2,1-c][1,4]benzothiazepin-12-one (28-3)

28-1 (56 mg, 0.135 mmol) was dissolved in toluene (5 mL) and heated to reflux overnight. The crude reaction mixture was concentrated to dryness and the crude residue was purified using silica gel chromatography (10-100% EtOAc/hex) to yield 30 mg of the racemic product. Chiral separation using ChiralPak IA, (5×25 cm, 5μ) 40:60:0.1 heptane:IPA:TFA, 50 ml/min afforded 28-2 and 28-3 ¹H NMR (400 MHz, CDCl₃): δ 8.15-8.10 (m, 2H); 7.60 (dd, J=7.4, 1.7 Hz, 1H); 7.47-7.33 (m, 3H); 7.23-7.14 (m, 2H); 5.11-5.05 (m, 1H); 3.91-3.83 (m, 1H); 3.44-3.30 (m, 1H); 3.21-2.94 (m, 2H); 2.18-2.12 (m, 1H); 2.07-1.78 (m, 3H); 1.25-1.14 (m, 1H). m/z (M+H) 396.1184 found, 396.1177 required.

### Example 30

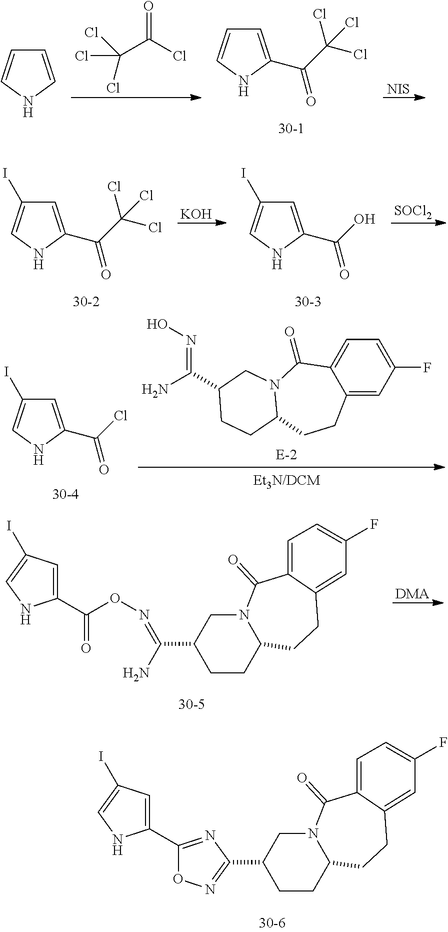

Intermediate E-2 was obtained following the same procedure described for Intermediate E-1, but starting from Intermediate D-8. D-8 is the resolved intermediate of D-5. m/z (M+H) 292.3 found, 292.3 required.

### 2,2,2-Trichloro-1-(1H-pyrrol-2-yl)ethanone (30-1)

Intermediate 30-1 was obtained following the same procedure described by Clark, B. R. and Murphy, C. D., Org. Biomol. Chem. 2009, 7, 111-116.

### 2,2,2-Trichloro-1-(4-iodo-1H-pyrrol-2-yl)ethanone (30-2)

To the solution of 30-1 (7.55 g, 35.5 mmol) in acetonitrile (300 ml) was added the N-idosuccinimide (8.39 g, 37.3 mmol). The resulting mixture was stirred for 4 hours at RT. The acetonitrile was taken off by rotavapor and the crude was dissolved in EtOAc. The organic solution was washed with brine, dried over MgSO₄, filtered and evaporated in vacuum. The crude was directly used for next step reaction without purification (30-2).

### 4-Iodo-1H-pyrrole-2-carboxylic acid (30-3)

The 30-2 (12 g, 35.5 mmol) in 2 M KOH (89 mL, 177 mmol) was stirred for 1 hour. To it was added the concentrate HCl (29.6 mL, 355 mmol). Solid formed. The solid was collected by filtration and washed with water to provide pure 30-3.

### 4-Iodo-1H-pyrrole-2-carbonyl chloride (30-4)

A solution of 30-3 (120 mg, 0.506 mmol) in CH₂Cl₂ (15 mL) was treated with thionyl chloride (0.370 mL, 5.06 mmol) and stirred at 60° C. for 6 h. After this time, the reaction mixture was evaporated in vacuum and re-evaporated from toluene to give 30-4. This material was used without further purification.

### (3S,12aS)-9-Fluoro-N′-{[(4-iodo-1H-pyrrol-2-yl)carbonyl]oxy}-6-oxo-1,2,3,4,6,11,12,12a-octahydropyrido[1,2-b][2]-benzazepine-3-carboximidamide (30-5)

A suspension of F-2 (50 mg, 0.172 mmol) in CH₂Cl₂ (4 mL) was treated with triethylamine (51 μL, 0.378 mmol) and stirred until mostly dissolved. This mixture was cooled in an ice bath and treated with a solution of 30-4 (66 mg, 0.257 mmol) in CH₂Cl₂ (4 mL) slowly over 5 min. The reaction mixture was stirred in an ice bath for 10 min and then stirred at 25° C. for 30 min. This mixture was diluted with water and extracted with CH₂Cl₂. The combined extracts were dried over MgSO₄, filtered and evaporated in vacuum. This residue was purified by column chromatography using RediSep column (12 g) eluting with a gradient of 0-5% MeOH/CH₂Cl₂ to give 30-5. m/z (M+H) 511.0 found, 511.3 required.

### (3S,12aS)-9-Fluoro-3-(4-iodo-1H-pyrrol-2-yl)-1,2,4-oxadiazol-3-1,3,4,11,12,12a-hexahydropyrido[1,2-b][2]benzazepin-6(2H)-one (30-6)

A solution of 30-5 (70 mg, 0.137 mmol) in DMA (3 mL) was placed in a 2-5 mL microwave reaction vial and microwaved for 60 min at 130° C. The reaction mixture was directly loaded to a Gilson reverse phase column (Phenomenex Axia C18(2) 100 A 10 u), and eluted with 5-95% MeCN/H₂O+0.1% TFA. The product containing fractions were lyophilized to yield 30-6. MS m/z (M+1) 493.0540 found, 493.0531 required.

### Example 31

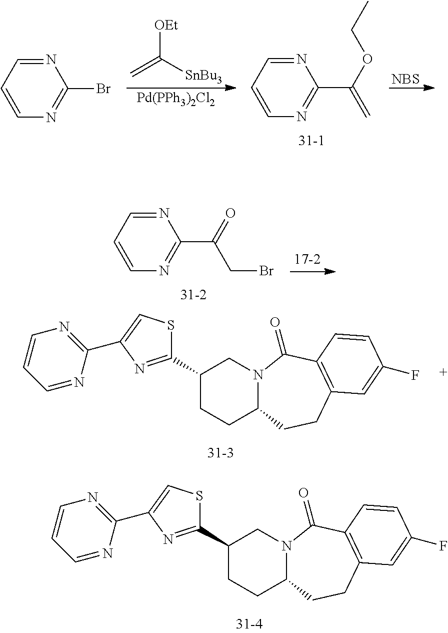

### 2-(1-Ethoxyethenyl pyrimidine (31-1)

A solution of 2-bromopyrimidine (770 mg, 4.84 mmol), tributyl(1-ethoxyvinyl)tin (1.80 mL, 5.33 mmol), and bis(triphenylphosphine)palladium(II) chloride (170 mg, 0.242 mmol) in dioxane (35 mL) was heated at reflux for 44 h. Additional tributyl(1-ethoxyvinyl)tin (1.80 mL, 5.33 mmol), and bis(triphenylphosphine)palladium(II) chloride (100 mg) were added after 26 h. The reaction mixture was poured onto sat. aq. NaHCO₃ (10 mL) and brine (30 mL) and extracted with EtOAc (3×30 mL). The combined extracts were dried over Na₂SO₄, filtered, and the solvent removed in vacuo. The crude product was purified by chromatography using silica gel (80 g) and eluting with a 3-100% gradient of EtOAc/Hexanes to provide 18-1. MS m/z (M+H) 152.1 found, 152.1 required.

### 2-Bromo-1-(pyrimidin-2-yl)ethanone (31-2)

NBS (521 mg, 2.93 mmol) was added to a solution of 18-1 (440 mg, 2.93 mmol) in THF (10 mL) and pH=7 phosphate buffer (1.7 mL) and the solution was stirred for 1 h at ambient temperature. The reaction mixture was poured onto pH=7 phosphate buffer (10 mL) and extracted with EtOAc (3×10 mL). The combined extracts were dried over Na₂SO₄, filtered, and the solvent removed in vacuo. The crude product was purified by chromatography using silica gel (80 g) and eluting with a 3-100% gradient of EtOAc/Hexanes to provide 18-2. MS m/z (M+H) 202.9 found, 202.9 required.

### (3S,12aS)-9-Fluoro-3-[4-(pyrimidin-2-yl)-1,3-thiazol-2-yl]-1,3,4,11,12,12a-hexahydropyrido[1,2-b][2]benzazepin-6(2H)-one (31-3) and (3R,12aS)-9-Fluoro-3-[4-(pyrimidin-2-yl)-1,3-thiazol-2-yl]-1,3,4,11,12,12a-hexahydropyrido[1,2-b][2]benzazepin-6(2H)-one (31-4)

A solution of 18-2 (256 mg, 1.27 mmol) and 17-2 (280 mg, 0.958 mmol) in EtOH (5 mL) was heated at 80° C. for 30 min. The reaction was poured onto sat. aq. NaHCO₃ (20 mL) and extracted with CH₂Cl₂ (3×20 mL). The combined extracts were dried over Na₂SO₄, filtered, and concentrated in vacuo. The crude product was purified by chromatography using silica gel (80 g) and eluting with a 3-100% gradient of EtOAc/CH₂Cl₂ to provide 18-3: MS m/z (M+H) 395.1340 found, 395.1336 required; and 18-4: MS m/z (M+H) 395.1344 found, 395.1336 required.

### Example 32

The following compounds were prepared using the foregoing methodology, but substituting the appropriately substituted reagent, as described in the foregoing Reaction Schemes and Examples. The requisite starting materials were commercially available, described in the literature or readily synthesized by one skilled in the art of organic synthesis without undue experimentation.

| Cmpd | Structure | Name | MS m/z (M + H) |
|---|---|---|---|
| 32-1 | 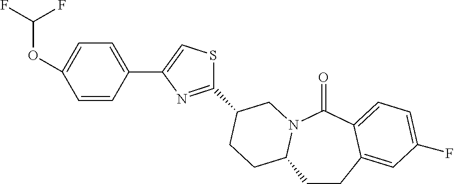 [SMILES: O=C1c2ccc(F)cc2CC[C@@H]2CC[C@H](c3nc(-c4ccc(OC(F)F)cc4)cs3)CN12] | (3S,12aS)-3-{4-[4- (difluoromethoxy)phenyl]- 1,3-thiazol-2-yl}-9- fluoro-1,3,4,11,12,12a- hexahydropyrido[1,2- b][2]benzazepin-6(2H)- one | 459.1345 found, 459.1349 required. |
| 32-2 | 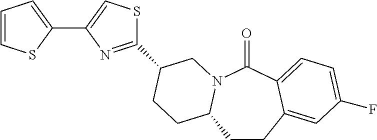 [SMILES: O=C1c2ccc(F)cc2CC[C@@H]2CC[C@H](c3nc(-c4cccs4)cs3)CN12] | (3S,12aS)-9-fluoro-3-[4- (thiophen-2-yl)-1,3- thiazol-2-yl]- 1,3,4,11,12,12a- hexahydropyrido[1,2- b][2]benzazepin-6(2H)- one | 399.0992 found, 399.0996 required. |
| 32-3 | 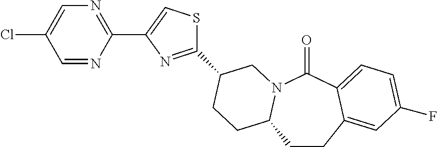 [SMILES: O=C1c2ccc(F)cc2CC[C@@H]2CC[C@H](c3nc(-c4ncc(Cl)cn4)cs3)CN12] | (3S,12aS)-3-[4-(5- chloropyrimidin-2-yl)- 1,3-thiazol-2-yl]-9- fluoro-1,3,4,11,12,12a- hexahydropyrido[1,2- b][2]benzazepin-6(2H)- one | 429.0955 found, 429.0947 required. |
| 32-4 | 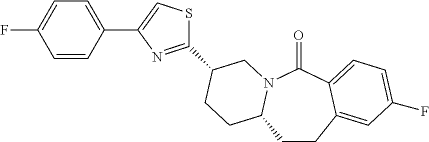 [SMILES: O=C1c2ccc(F)cc2CC[C@@H]2CC[C@H](c3nc(-c4ccc(F)cc4)cs3)CN12] | (3S,12aS)-9-fluoro-3-[4- (4-fluorophenyl)-1,3- thiazol-2-yl]- 1,3,4,11,12,12a- hexahydropyrido[1,2- b][2]benzazepin-6(2H)- one | 411.1341 found, 411.1337 required. |
| 32-5 | 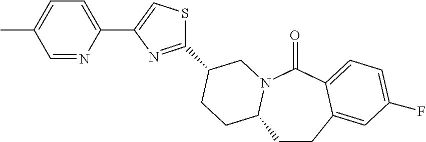 [SMILES: Cc1ccc(-c2csc([C@H]3CC[C@H]4CCc5cc(F)ccc5C(=O)N4C3)n2)nc1] | (3S,12aS)-9-fluoro-3-[4- (5-methylpyridin-2-yl)- 1,3-thiazol-2-yl]- 1,3,4,11,12,12a- hexahydropyrido[1,2- b][2]benzazepin-6(2H)- one | 408.1549 found, 408.1540 required. |
| 32-6 | 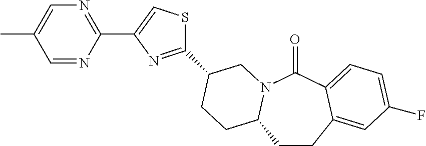 [SMILES: Cc1cnc(-c2csc([C@H]3CC[C@H]4CCc5cc(F)ccc5C(=O)N4C3)n2)nc1] | (3S,12aS)-9-fluoro-3-[4- (5-methylpyrimidin-2- yl)-1,3-thiazol-2-yl]- 1,3,4,11,12,12a- hexahydropyrido[1,2- b][2]benzazepin-6(2H)- one | 409.1497 found, 409.1493 required. |
| 32-7 | 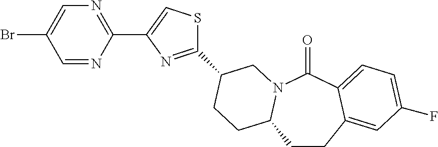 [SMILES: O=C1c2ccc(F)cc2CC[C@@H]2CC[C@H](c3nc(-c4ncc(Br)cn4)cs3)CN12] | (3S,12a,S)-3-[4-(5- bromopyrimidin-2-yl)- 1,3-thiazol-2-yl]-9- fluoro-1,3,4,11,12,12a- hexahydropyrido[1,2- b][2]benzazepin-6(2H)- one | 473.0457 found, 473.0441 required. |
| 32-8 | 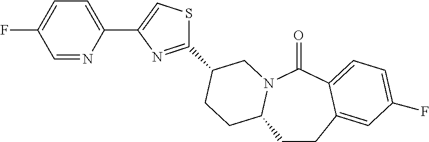 [SMILES: O=C1c2ccc(F)cc2CC[C@@H]2CC[C@H](c3nc(-c4ccc(F)cn4)cs3)CN12] | (3S,12aS)-9-fluoro-3-[4- (5-fluoropyridin-2-yl)- 1,3-thiazol-2-yl]- 1,3,4,11,12,12a- hexahydropyrido[1,2- b][2]benzazepin-6(2H)- one | 412.1289 found, 412.1290 required. |
| 32-9 | 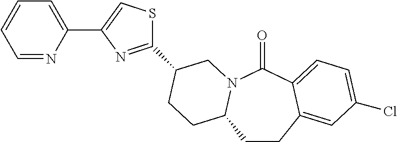 [SMILES: O=C1c2ccc(Cl)cc2CC[C@@H]2CC[C@H](c3nc(-c4ccccn4)cs3)CN12] | (3S,12aS)-9-chloro-3-[4- (pyridin-2-yl)-1,3- thiazol-2-yl]- 1,3,4,11,12,12a- hexahydropyrido[1,2- b][2]benzazepin-6(2H)- one | 410.1096 found, 410.1088 required. |
| 32-10 | 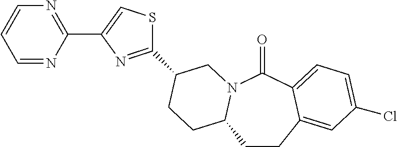 [SMILES: O=C1c2ccc(Cl)cc2CC[C@@H]2CC[C@H](c3nc(-c4ncccn4)cs3)CN12] | (3S,12aS)-9-chloro-3-[4- (pyrimidin-2-yl)-1,3- thiazol-2-yl]- 1,3,4,11,12,12a- hexahydropyrido[1,2- b][2]benzazepin-6(2H)- one | 411.1046 found, 411.1041 required. |
| 32-11 | 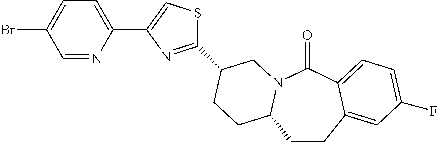 [SMILES: O=C1c2ccc(F)cc2CC[C@@H]2CC[C@H](c3nc(-c4ccc(Br)cn4)cs3)CN12] | (3S,12aS)-3-[4-(5- bromopyridin-2-yl)-1,3- thiazol-2-yl]-9-fluoro- 1,3,4,11,12,12a- hexahydropyrido[1,2- b][2]benzazepin-6(2H)- one | 472.0502 found, 472.0489 required. |
| 32-12 | 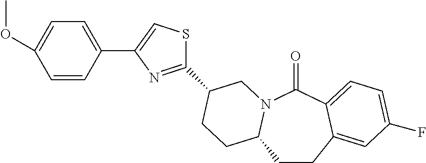 [SMILES: COc1ccc(-c2csc([C@H]3CC[C@H]4CCc5cc(F)ccc5C(=O)N4C3)n2)cc1] | (3S,12aS)-9-fluoro-3-[4- (4-methoxyphenyl)-1,3- thiazol-2-yl]- 1,3,4,11,12,12a- hexahydropyrido[1,2- b][2]benzazepin-6(2H)- one | 423.1533 found, 423.1537 required. |
| 32-13 | 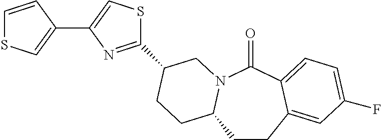 [SMILES: O=C1c2ccc(F)cc2CC[C@@H]2CC[C@H](c3nc(-c4ccsc4)cs3)CN12] | (3S,12aS)-9-fluoro-3-[4- (thiophen-3-yl)-1,3- thiazol-2-yl]- 1,3,4,11,12,12a- hexahydropyrido[1,2- b][2]benzazepin-6(2H)- one | 399.0996 found, 399.0996 required. |
| 32-14 | 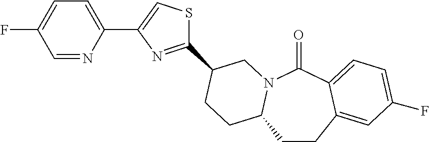 [SMILES: O=C1c2ccc(F)cc2CC[C@@H]2CC[C@@H](c3nc(-c4ccc(F)cn4)cs3)CN12] | (3R,12aS)-9-fluoro-3-[4- (5-fluoropyridin-2-yl)- 1,3-thiazol-2-yl]- 1,3,4,11,12,12a- hexahydropyrido[1,2- b][2]benzazepin-6(2H)- one | 412.1296 found, 412.1290 required. |
| 32-15 | 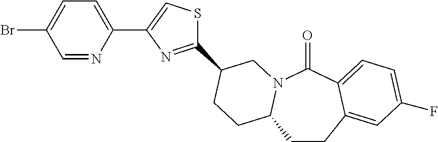 [SMILES: O=C1c2ccc(F)cc2CC[C@@H]2CC[C@@H](c3nc(-c4ccc(Br)cn4)cs3)CN12] | (3R,12aS)-3-[4-(5- bromopyridin-2-yl)-1,3- thiazol-2-yl]-9-fluoro- 1,3,4,11,12,12a- hexahydropyrido[1,2- b][2]benzazepin-6(2H)- one | 472.0490 found, 472.0489 required. |
| 32-16 | 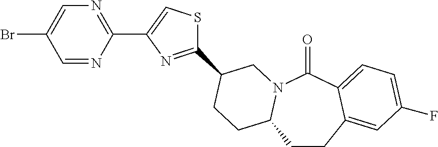 [SMILES: O=C1c2ccc(F)cc2CC[C@@H]2CC[C@@H](c3nc(-c4ncc(Br)cn4)cs3)CN12] | (3R,12aS)-3-[4-(5- bromopyrimidin-2-yl)- 1,3-thiazol-2-yl]-9- fluoro-1,3,4,11,12,12a- hexahydropyrido[1,2- b][2]benzazepin-6(2H)- one | 473.0462 found, 473.0441 required. |
| 32-17 | 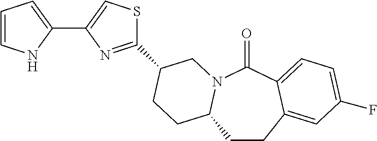 [SMILES: O=C1c2ccc(F)cc2CC[C@@H]2CC[C@H](c3nc(-c4ccc[nH]4)cs3)CN12] | (3S,12aS)-9-fluoro-3-[4- (1H-pyrrol-2-yl)-1,3- thiazol-2-yl]- 1,3,4,11,12,12a- hexahydropyrido[1,2- b][2]benzazepin-6(2H)- one | 382.1379 found, 382.1384 required. |
| 32-18 | 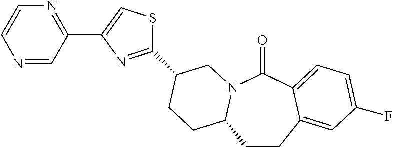 [SMILES: O=C1c2ccc(F)cc2CC[C@@H]2CC[C@H](c3nc(-c4cnccn4)cs3)CN12] | (3S,12aS)-9-fluoro-3-[4- (pyrazin-2-yl)-1,3- thiazol-2-yl]- 1,3,4,11,12,12a- hexahydropyrido[1,2- b][2]benzazepin-6(2H)- one | 395.1340 found, 395.1336 required. |
| 32-19 | 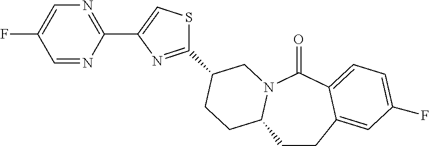 [SMILES: O=C1c2ccc(F)cc2CC[C@@H]2CC[C@H](c3nc(-c4ncc(F)cn4)cs3)CN12] | (3S,12aS)-9-fluoro-3-[4- (5-fluoropyrimidin-2- yl)-1,3-thiazol-2-yl]- 1,3,4,11,12,12a- hexahydropyrido[1,2- b][2]benzazepin-6(2H)- one | 413.1245 found, 413.1242 required. |
| 32-20 | 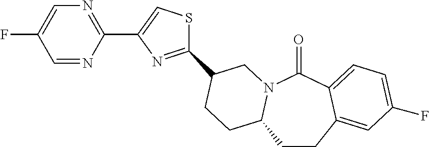 | (3R,12aS)-9-fluoro-3-[4- (5-fluoropyrimidin-2- yl)-1,3-thiazol-2-yl]- 1,3,4,11,12,12a- hexahydropyrido[1,2- b][2]benzazepin-6(2H)- one | 413.1250 found, 413.1242 required. |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound of the formula I: 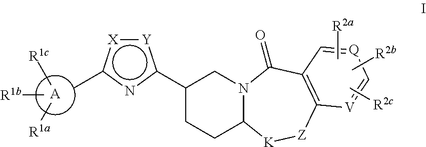
wherein:
A is selected from the group consisting of phenyl, naphthyl and heteroaryl;
X is selected from N, O, S and C(R⁸),
Y is selected from N, O, S and C(R⁸),
wherein X is N and Y is O, to form a oxadiazole ring, or
X is O and Y is N, to form a oxadiazole ring, or
X is C(R⁸) and Y is S to form a thiazole ring, or
X is S and Y is C(R⁸) to form a thiazole ring, or
X is C(R⁸) and Y is N to form an imidazole ring, or
X is N and Y is C(R⁸) to form an imidazole ring, or
X is C(R⁸) and Y is O to form an oxazole ring, or
X is O and Y is C(R⁸) to form an oxazole ring, or
X is N and Y is N to form a triazole ring;
Q is selected from C and N,
V is selected from C and N,
K is —CH₂—,
Z is selected from —CH₂—, —CH(CH₃)—, —CHF—, —CF₂—, —CH(OH)—, —O—, —S—, —S(O)—, —NH—, and —N(CH₃)—, or
K is —CH(OH)— and Z is —CH(OH)—, or
K and Z taken together form —CH═CH—, or
K and Z taken together form a cyclopropyl ring;
R^{1a}, R^{1b} and R^{1c} may be absent if the valency of A does not permit such substitution and are independently selected from the group consisting of:
(1) hydrogen,
(2) halogen,
(3) hydroxyl,
(4) —(C═O)ₘ—Oₙ—C₁₋₆alkyl, where m is 0 or 1, n is 0 or 1 (wherein if m is 0 or n is 0, a bond is present) and where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘ—C₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heteroaryl, where the heteraryl is unsubstituted or substituted with one or more substituents selected from R¹³,
(10) —(C═O)ₘ—NR¹⁰R¹¹, wherein R¹⁰ and R¹¹ are independently selected from the group consisting of:
(a) hydrogen,
(b) C₁₋₆alkyl, which is unsubstituted or substituted with R¹⁴,
(c) C₃₋₆alkenyl, which is unsubstituted or substituted with R¹⁴,
(d) C₃₋₆alkynyl, which is unsubstituted or substituted with R¹⁴,
(e) C₃₋₆cycloalkyl which is unsubstituted or substituted with R¹⁴,
(f) phenyl, which is unsubstituted or substituted with R¹⁴, and
(g) heteroaryl, which is unsubstituted or substituted with R¹⁴,
(11) —S(O)₂—NR¹⁰R¹¹,
(12) —S(O)_{q}—R¹², where q is 0, 1 or 2 and where R¹² is selected from the definitions of R¹⁰ and R¹¹,
(13) —CO₂H,
(14) —CN, and
(15) —NO₂;
R^{2a}, R^{2b} and R^{2c} are independently selected from the group consisting of:
(1) hydrogen,
(2) halogen,
(3) hydroxyl,
(4) —(C═O)ₘ—Oₙ—C₁₋₆alkyl, where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘ—C₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heteroaryl, where the heteroaryl is unsubstituted or substituted with one or more substituents selected from R¹³,
(10) —(C═O)ₘ—NR¹⁰R¹¹,
(11) —S(O)₂—NR¹⁰R¹¹,
(12) —S(O)_{q}—R¹²,
(13) —CO₂H,
(14) —CN, and
(15) —NO₂;
R⁸ is selected from the group consisting of:
(1) hydrogen,
(2) halogen,
(3) C₁₋₆alkyl, which is unsubstituted or substituted with R¹³,
(4) C₃₋₆alkenyl, which is unsubstituted or substituted with R¹³,
(5) C₃₋₆alkynyl, which is unsubstituted or substituted with R¹³,
(6) C₃₋₆cycloalkyl which is unsubstituted or substituted with R¹³,
(7) phenyl, which is unsubstituted or substituted with R¹³, and
(6) heteroaryl, which is unsubstituted or substituted with R¹³,
R¹³ is selected from the group consisting of:
(1) halogen,
(2) hydroxyl,
(3) —(C═O)ₘ—Oₙ—C₁₋₆alkyl, where the alkyl is unsubstituted or substituted with one or more substituents selected from R¹⁴,
(4) —Oₙ—(C₁₋₃)perfluoroalkyl,
(5) —(C═O)ₘ—Oₙ—C₃₋₆cycloalkyl, where the cycloalkyl is unsubstituted or substituted with one or more substituents selected from R¹⁴,
(6) —(C═O)ₘ—C₂₋₄alkenyl, where the alkenyl is unsubstituted or substituted with one or more substituents selected from R¹⁴,
(7) —(C═O)ₘ—C₂₋₄alkynyl, where the alkynyl is unsubstituted or substituted with one or more substituents selected from R¹⁴,
(8) —(C═O)ₘ—Oₙ-phenyl or —(C═O)ₘ—Oₙ-napthyl, where the phenyl or naphthyl is unsubstituted or substituted with one or more substituents selected from R¹⁴,
(9) —(C═O)ₘ—Oₙ-heteroaryl, where the heteroaryl is unsubstituted or substituted with one or more substituents selected from R¹⁴,
(10) —(C═O)ₘ—NR¹⁰R¹¹,
(11) —S(O)₂—NR¹⁰R¹¹,
(12) —S(O)_{q}—R¹²,
(13) —CO₂H,
(14) —CN, and
(15) —NO₂;
R¹⁴ is selected from the group consisting of:
(1) hydroxyl,
(2) halogen,
(3) C₁₋₆alkyl,
(4) —C₃₋₆cycloalkyl,
(5) —O—C₁₋₆ alkyl,
(6) —O(C═O)—C₁₋₆alkyl,
(7) —NH—C₁₋₆ alkyl,
(8) phenyl,
(9) heteroaryl,
(10) —CO₂H, and
(11) —CN;
or a pharmaceutically acceptable salt thereof.

2. The compound of claim 1 of the formula Ia: 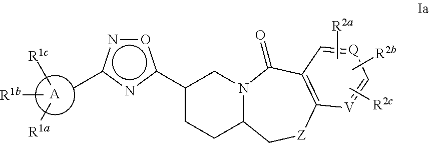
or a pharmaceutically acceptable salt thereof.

3. The compound of claim 1 of the formula Ib: 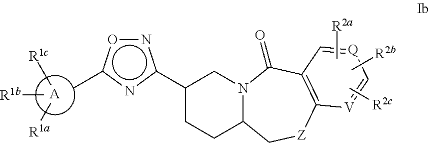
or a pharmaceutically acceptable salt thereof.

4. The compound of claim 1 of the formula Ic: 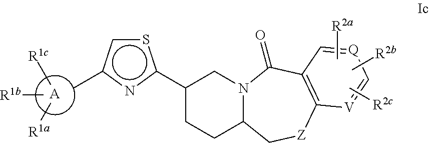
or a pharmaceutically acceptable salt thereof.

5. The compound of claim 1, or a pharmaceutically acceptable salt thereof, wherein A is selected from the group consisting of phenyl, pyridyl and pyrrolyl.

6. The compound of claim 1, or a pharmaceutically acceptable salt thereof, wherein Q is C and V is C.

7. The compound of claim 1, or a pharmaceutically acceptable salt thereof, wherein Z is —CH₂—.

8. The compound of claim 1, or a pharmaceutically acceptable salt thereof, wherein R^{1a}, R^{1b} and R^{1c} are independently selected from the group consisting of:
(1) hydrogen,
(2) halogen,
(3) hydroxyl,
(4) C₁₋₆alkyl, which is unsubstituted or substituted with halogen, hydroxyl, phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆ alkyl or —NO₂,
(7) phenyl, which is unsubstituted or substituted with halogen, hydroxyl, C₁₋₆alkyl, —O—C₁₋₆ alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, or —O—C₁₋₆alkyl.

9. The compound of claim 1, or a pharmaceutically acceptable salt thereof, wherein R^{1a}, R^{1b} and R^{1c} are independently selected from the group consisting of:
(1) hydrogen,
(2) halogen, and
(3) C₁₋₆alkyl.

10. The compound of claim 1, or a pharmaceutically acceptable salt thereof, wherein R^{2a}, R^{2b} and R^{2c} are independently selected from the group consisting of:
(1) hydrogen,
(2) halogen,
(3) hydroxyl,
(4) C₁₋₆alkyl, which is unsubstituted or substituted with halogen, hydroxyl or phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆ alkyl, —O—C₁₋₆ alkyl or —NO₂,
(7) phenyl, which is unsubstituted or substituted with halogen, hydroxyl, C₁₋₆alkyl, —O—C₁₋₆ alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, or —O—C₁₋₆alkyl.

11. The compound of claim 1, or a pharmaceutically acceptable salt thereof, wherein R^{2a}, R^{2b} and R^{2c} are independently selected from the group consisting of:
(1) hydrogen,
(2) chloro,
(3) fluoro,
(4) bromo,
(5) methoxy,
(6) t-butoxy,
(7) difluoromethyl, and
(8) trifluoromethyl.

12. A compound which is selected from the group consisting of:
3-(3-(4-methyl-1H-pyrrol-2-yl)-1,2,4-oxadiazol-5-yl)-1,3,4,11,12,12a-hexahydrobenzo[e]pyrido[1,2-a]azepin-6(2H)-one;
cis-(3S,12aS)-3-(3-(4-methyl-1H-pyrrol-2-yl)-1,2,4-oxadiazol-5-yl)-1,3,4,11,12,12a-hexahydrobenzo[e]pyrido[1,2-a]azepin-6(2H)-one;
cis-(3R,12aR)₃-(3-(4-methyl-1H-pyrrol-2-yl)-1,2,4-oxadiazol-5-yl)-1,3,4,11,12,12a-hexahydrobenzo[e]pyrido[1,2-a]azepin-6(2H)-one;
9-Fluoro-3-[3-(4-methyl-1H-pyrrol-2-yl)-1,2,4-oxadiazol-5-yl]-1,3,4,11,12,12a-hexahydropyrido[1,2-b][2]benzazepin-6(2H)-one;
(3S,12aS)-9-fluoro-3-[3-(4-methyl-1H-pyrrol-2-yl)-1,2,4-oxadiazol-5-yl]-1,3,4,11,12,12a-hexahydropyrido[1,2-b][2]benzazepin-6(2H)-one;
9-fluoro-3-[3-(4-chloro1H-pyrrol-2-yl)-1,2,4-oxadiazol-5-yl]-1,3,4,11,12,12a-hexahydropyrido[1,2-b][2]benzazepin-6(2H)-one;
(3S,12aS)-9-fluoro-3-[3-(4-chloro1H-pyrrol-2-yl)-1,2,4-oxadiazol-5-yl]-1,3,4,11,12,12a-hexahydropyrido[1,2-b][2]benzazepin-6(2H)-one;
9-fluoro-3-(4-(5-fluoropyridin-2-yl)oxazol-2-yl)-1,3,4,11,12,12a-hexahydrobenzo[e]pyrido[1,2-a]azepin-6(2H)-one;
cis-(3S,12aS)-9-fluoro-3-(4-(5-fluoropyridin-2-yl)oxazol-2-yl)-1,3,4,11,12,12a-hexahydrobenzo[e]pyrido[1,2-a]azepin-6(2H)-one;
cis-(3R,12aR)-9-fluoro-3-(4-(5-fluoropyridin-2-yl)oxazol-2-yl)-1,3,4,11,12,12a-hexahydrobenzo[e]pyrido[1,2-a]azepin-6(2H)-one;
9-fluoro-3-(5-(5-fluoropyridin-2-yl)-1,2,4-oxadiazol-3-yl)-1,3,4,11,12,12a-hexahydrobenzo[e]pyrido[1,2-a]azepin-6(2H)-one;
cis-(3S,12aS)-9-fluoro-3-(5-(5-fluoropyridin-2-yl)-1,2,4-oxadiazol-3-yl)-1,3,4,11,12,12a-hexahydrobenzo[e]pyrido[1,2-a]azepin-6(2H)-one;
cis-(3R,12aR)-9-fluoro-3-(5-(5-fluoropyridin-2-yl)-1,2,4-oxadiazol-3-yl)-1,3,4,11,12,12a-hexahydrobenzo[e]pyrido[1,2-a]azepin-6(2H)-one;
8-(3-(4-chloro-1H-pyrrol-2-yl)-1,2,4-oxadiazol-5-yl)-3-fluoro-8,9,10,10a,11,12-hexahydrodipyrido[1,2-a:3′,4′-e]azepin-5(7H)-one;
(±) trans-8-(3-(4-chloro-1H-pyrrol-2-yl)-1,2,4-oxadiazol-5-yl)-3-fluoro-8,9,10,10a,11,12-hexahydrodipyrido[1,2-a:3′,4′-e]azepin-5(7H)-one;
cis-(8S,10aS)-8-(3-(4-chloro-1H-pyrrol-2-yl)-1,2,4-oxadiazol-5-yl)-3-fluoro-8,9,10,10a,11,12-hexahydrodipyrido[1,2-a:3′,4′-e]azepin-5(7H)-one;
cis-(8R,10aR)-8-(3-(4-chloro-1H-pyrrol-2-yl)-1,2,4-oxadiazol-5-yl)-3-fluoro-8,9,10,10a,11,12-hexahydrodipyrido[1,2-a:3′,4′-e]azepin-5(7H)-one;
3-fluoro-8-(3-(4-methyl-1H-pyrrol-2-yl)-1,2,4-oxadiazol-5-yl)-8,9,10,10a-tetrahydrodipyrido[1,2-a:3′,4′-e]azepin-5(7H)-one;
(±) cis-3-fluoro-8-(3-(4-methyl-1H-pyrrol-2-yl)-1,2,4-oxadiazol-5-yl)-8,9,10,10a-tetrahydrodipyrido[1,2-a:3′,4′-e]azepin-5(7H)-one;
(±) cis-(Z)-3-[3-(4-methyl-1H-pyrrol-2-yl)-1,2,4-oxadiazol-5-yl]-1,3,4,12a-tetrahydropyrido[1,2-b][2]benzazepin-6(2H)-one;
9-[3-(4-chloro-1H-pyrrol-2-yl)-1,2,4-oxadiazol-5-yl]-12-oxo-5,6,6a,7,8,9,10,12-octahydropyrido[2,1-c][1,4]benzodiazepine;
(6aS,9S)-9-[3-(4-chloro-1H-pyrrol-2-yl)-1,2,4-oxadiazol-5-yl]-12-oxo-5,6,6a,7,8,9,10,12-octahydropyrido[2,1-c][1,4]benzodiazepine;
9-[3-(4-chloro-1H-pyrrol-2-yl)-1,2,4-oxadiazol-5-yl]-6,6a,7,8,9,10-hexahydro-12H-pyrido[2,1-c][1,4]benzoxazepin-12-one;
(6aS,9S)-9-[3-(4-chloro-1H-pyrrol-2-yl)-1,2,4-oxadiazol-5-yl]-6,6a,7,8,9,10-hexahydro-12H-pyrido[2,1-c][1,4]benzoxazepin-12-one;
(6aR,9R)-9-[3-(4-chloro-1H-pyrrol-2-yl)-1,2,4-oxadiazol-5-yl]-6,6a,7,8,9,10-hexahydro-12H-pyrido[2,1-c][1,4]benzoxazepin-12-one;
2-chloro-8-[3-(4-chloro-1H-pyrrol-2-yl)-1,2,4-oxadiazol-5-yl]-8,9,10,10a,11,12-hexahydrodipyrido[1,2-a:2′,3′-e]azepin-5(7H)-one;
(8S,10aS)-2-chloro-8-[3-(4-chloro-1H-pyrrol-2-yl)-1,2,4-oxadiazol-5-yl]-8,9,10,10a,11,12-hexahydrodipyrido[1,2-a:2′,3′-e]azepin-5(7H)-one;
(8R,10aR)-2-chloro-8-[3-(4-chloro-1H-pyrrol-2-yl)-1,2,4-oxadiazol-5-yl]-8,9,10,10a,11,12-hexahydrodipyrido[1,2-a:2′,3′-e]azepin-5(7H)-one;
8-[3-(4-chloro-1H-pyrrol-2-yl)-1,2,4-oxadiazol-5-yl]-2-methoxy-8,9,10,10a,11,12-hexahydrodipyrido[1,2-a:2′,3′-e]azepin-5(7H)-one;
(8S,10aS)-8-[3-(4-chloro-1H-pyrrol-2-yl)-1,2,4-oxadiazol-5-yl]-2-methoxy-8,9,10,10a,11,12-hexahydrodipyrido[1,2-a:2′,3′-e]azepin-5(7H)-one;
8-[3-(4-chloro-1H-pyrrol-2-yl)-1,2,4-oxadiazol-5-yl]-2-fluoro-8,9,10,10a,11,12-hexahydrodipyrido[1,2-a:2′,3′-e]azepin-5(7H)-one;
(8S,10aS)-8-[3-(4-chloro-1H-pyrrol-2-yl)-1,2,4-oxadiazol-5-yl]-2-fluoro-8,9,10,10a,11,12-hexahydrodipyrido[1,2-a:2′,3′-e]azepin-5(7H)-one;
2-fluoro-8-[3-(4-methyl-1H-pyrrol-2-yl)-1,2,4-oxadiazol-5-yl]-8,9,10,10a,11,12-hexahydrodipyrido[1,2-a:2′,3′-e]azepin-5(7H)-one;
(8S,10aS)-2-fluoro-8-[3-(4-methyl-1H-pyrrol-2-yl)-1,2,4-oxadiazol-5-yl]-8,9,10,10a,11,12-hexahydrodipyrido[1,2-a:2′,3′-e]azepin-5(7H)-one;
2-fluoro-8-[3-(5-fluoropyridin-2-yl)-1,2,4-oxadiazol-5-yl]-8,9,10,10a,11,12-hexahydrodipyrido[1,2-a:2′,3′-e]azepin-5(7H)-one;
(8S,10aS)-2-fluoro-8-[3-(5-fluoropyridin-2-yl)-1,2,4-oxadiazol-5-yl]-8,9,10,10a,11,12-hexahydrodipyrido[1,2-a:2′,3′-e]azepin-5(7H)-one;
9-fluoro-3-[4-(pyridin-2-yl)-1,3-thiazol-2-yl]-1,3,4,11,12,12a-hexahydropyrido[1,2-b][2]benzazepin-6(2H)-one;
(3S,12aS)-9-fluoro-3-[4-(pyridin-2-yl)-1,3-thiazol-2-yl]-1,3,4,11,12,12a-hexahydropyrido[1,2-b][2]benzazepin-6(2H)-one;
(3R,12aS)-9-fluoro-3-[4-(pyridin-2-yl)-1,3-thiazol-2-yl]-1,3,4,11,12,12a-hexahydropyrido[1,2-b][2]benzazepin-6(2H)-one;
9-cyano-3-[3-(4-chloro1H-pyrrol-2-yl)-1,2,4-oxadiazol-5-yl]-1,3,4,11,12,12a-hexahydropyrido[1,2-b][2]benzazepin-6(2H)-one;
(±) cis-9-cyano-3-[3-(4-chloro1H-pyrrol-2-yl)-1,2,4-oxadiazol-5-yl]-1,3,4,11,12,12a-hexahydropyrido[1,2-b][2]benzazepin-6(2H)-one;
10-methoxy-3-[3-(4-methyl-1H-pyrrol-2-yl)-1,2,4-oxadiazol-5-yl]-1,3,4,11,12,12a-hexahydropyrido[1,2-b][2]benzazepin-6(2H)-one;
(±) cis-10-methoxy-3-[3-(4-methyl-1H-pyrrol-2-yl)-1,2,4-oxadiazol-5-yl]-1,3,4,11,12,12a-hexahydropyrido[1,2-b][2]benzazepin-6(2H)-one;
9,11,11-trifluoro-3-[3-(5-fluoropuridin-2-yl)-1,2-4-oxadiazol-5-yl}-1,3,4,11,12,12a-hexahydropyrido[1,2-b][2]benzazepin-6(2H)-one;
(±) cis-9,11,11-trifluoro-3-[3-(5-fluoropuridin-2-yl)-1,2-4-oxadiazol-5-yl}-1,3,4,11,12,12a-hexahydropyrido[1,2-b][2]benzazepin-6(2H)-one;
9,11,11-trifluoro-3-[3-(4-methyl-1H-pyrrol-2-yl)-1,2,4-oxadiazol-5-yl]-1,3,4,11,12,12a-hexahydropyrido[1,2-b][2]benzazepin-6(2H)-one;
(±)cis-9,11,11-trifluoro-3-[3-(4-methyl-1H-pyrrol-2-yl)-1,2,4-oxadiazol-5-yl]-1,3,4,11,12,12a-hexahydropyrido[1,2-b][2]benzazepin-6(2H)-one;
9-fluoro-3-[3-(5-fluoropyridin-2-yl)-1,2-4-oxadiazol-5-yl}-11,12-dihydroxy-1,3,4,11,12,12a-hexahydropyrido[1,2-b][2]benzazepin-6(2H)-one;
(±) cis-9-fluoro-3-[3-(5-fluoropyridin-2-yl)-1,2-4-oxadiazol-5-yl}-11,12-dihydroxy-1,3,4,11,12,12a-hexahydropyrido[1,2-b][2]benzazepin-6(2H)-one;
9-[3-(4-chloro-1H-pyrrol-2-yl)-1,2,4-oxadiazol-5-yl]-3-fluoro-6,6a,7,8,9,10-hexahydrodipyrido[1,2-a:4′,3′-e]azepin-12(5H)-one;
cis-9-[3-(4-chloro-1H-pyrrol-2-yl)-1,2,4-oxadiazol-5-yl]-3-fluoro-6,6a,7,8,9,10-hexahydrodipyrido[1,2-a:4′,3′-e]azepin-12(5H)-one;
(6aS,9S)-9-[3-(4-chloro-1H-pyrrol-2-yl)-1,2,4-oxadiazol-5-yl]-3-fluoro-6,6a,7,8,9,10-hexahydrodipyrido[1,2-a:4′,3′-e]azepin-12(5H)-one;
(6aR,9R)-9-[3-(4-chloro-1H-pyrrol-2-yl)-1,2,4-oxadiazol-5-yl]-3-fluoro-6,6a,7,8,9,10-hexahydrodipyrido[1,2-a:4′,3′-e]azepin-12(5H)-one;
3-fluoro-9-[5-(5-fluoropyridin-2-yl)-1,2,4-oxadiazol-3-yl]-6,6a,7,8,9,10-hexahydrodipyrido[1,2-a:4′,3′-e]azepin-12(5H)-one;
cis-3-fluoro-9-[5-(5-fluoropyridin-2-yl)-1,2,4-oxadiazol-3-yl]-6,6a,7,8,9,10-hexahydrodipyrido[1,2-a:4′,3′-e]azepin-12(5H)-one;
(6aS,9S)-3-fluoro-9-[5-(5-fluoropyridin-2-yl)-1,2,4-oxadiazol-3-yl]-6,6a,7,8,9,10-hexahydrodipyrido[1,2-a:4′,3′-e]azepin-12(5H)-one;
(6aR,9R)-3-fluoro-9-[5-(5-fluoropyridin-2-yl)-1,2,4-oxadiazol-3-yl]-6,6a,7,8,9,10-hexahydrodipyrido[1,2-a:4′,3′-e]azepin-12(5H)-one;
9-[5-(4-chloro-1H-pyrrol-2-yl)-1,2,4-oxadiazol-3-yl]-3-fluoro-6,6a,7,8,9,10-hexahydrodipyrido[1,2-a:4′,3′-e]azepin-12(5H)-one;
(6aS,9S)-9-[5-(4-chloro-1H-pyrrol-2-yl)-1,2,4-oxadiazol-3-yl]-3-fluoro-6,6a,7,8,9,10-hexahydrodipyrido[1,2-a:4′,3′-e]azepin-12(5H)-one;
(6aR,9R)-9-[5-(4-chloro-1H-pyrrol-2-yl)-1,2,4-oxadiazol-3-yl]-3-fluoro-6,6a,7,8,9,10-hexahydrodipyrido[1,2-a:4′,3′-e]azepin-12(5H)-one;
9-[5-(4-chloro-1H-pyrrol-2-yl)-1,2,4-oxadiazol-3-yl]-6,6a,7,8,9,10-hexahydro-12H-pyrido[2,1-c][1,4]benzothiazepin-12-one;
(6aS,9S)-9-[5-(4-chloro-1H-pyrrol-2-yl)-1,2,4-oxadiazol-3-yl]-6,6a,7,8,9,10-hexahydro-12H-pyrido[2,1-c][1,4]benzothiazepin-12-one;
(6aR,9R)-9-[5-(4-chloro-1H-pyrrol-2-yl)-1,2,4-oxadiazol-3-yl]-6,6a,7,8,9,10-hexahydro-12H-pyrido[2,1-c][1,4]benzothiazepin-12-one;
(6aS,9S)-9-[5-(4-chloro-1H-pyrrol-2-yl)-1,2,4-oxadiazol-3-yl]-6,6a,7,8,9,10-hexahydro-12H-pyrido[2,1-c][1,4]benzothiazepin-12-one 5-oxide;
9-[5-(4-methyl-1H-pyrrol-2-yl)-1,2,4-oxadiazol-3-yl]-6,6a,7,8,9,10-hexahydro-12H-pyrido[2,1-c][1,4]benzothiazepin-12-one;
(6aS,9S)-9-[5-(4-methyl-1H-pyrrol-2-yl)-1,2,4-oxadiazol-3-yl]-6,6a,7,8,9,10-hexahydro-12H-pyrido[2,1-c][1,4]benzothiazepin-12-one;
(6aR,9R)-9-[5-(4-methyl-1H-pyrrol-2-yl)-1,2,4-oxadiazol-3-yl]-6,6a,7,8,9,10-hexahydro-12H-pyrido[2,1-c][1,4]benzothiazepin-12-one;
9-[5-(4-fluorophenyl)-1,2,4-oxadiazol-3-yl]-6,6a,7,8,9,10-hexahydro-12H-pyrido[2,1-c][1,4]benzothiazepin-12-one;
(6aS,9S)-9-[5-(4-fluorophenyl)-1,2,4-oxadiazol-3-yl]-6,6a,7,8,9,10-hexahydro-12H-pyrido[2,1-c][1,4]benzothiazepin-12-one;
(6aR,9R)-9-[5-(4-fluorophenyl)-1,2,4-oxadiazol-3-yl]-6,6a,7,8,9,10-hexahydro-12H-pyrido[2,1-c][1,4]benzothiazepin-12-one;
9-fluoro-3-[5-(4-iodo-1H-pyrrol-2-yl)-1,2,4-oxadiazol-3-yl]-1,3,4,11,12,12a-hexahydropyrido[1,2-b][2]benzazepin-6(2H)-one;
(3S,12aS)-9-fluoro-3-[5-(4-iodo-1H-pyrrol-2-yl)-1,2,4-oxadiazol-3-yl]-1,3,4,11,12,12a-hexahydropyrido[1,2-b][2]benzazepin-6(2H)-one;
9-fluoro-3-[4-(pyrimidin-2-yl)-1,3-thiazol-2-yl]-1,3,4,11,12,12a-hexahydropyrido[1,2-b][2]benzazepin-6(2H)-one;
(3S,12aS)-9-fluoro-3-[4-(pyrimidin-2-yl)-1,3-thiazol-2-yl]-1,3,4,11,12,12a-hexahydropyrido[1,2-b][2]benzazepin-6(2H)-one;
(3R,12aS)-9-fluoro-3-[4-(pyrimidin-2-yl)-1,3-thiazol-2-yl]-1,3,4,11,12,12a-hexahydropyrido[1,2-b][2]benzazepin-6(2H)-one;
3-{4-[4-(difluoromethoxy)phenyl]-1,3-thiazol-2-yl}-9-fluoro-1,3,4,11,12,12a-hexahydropyrido[1,2-b][2]benzazepin-6(2H)-one;
(3S,12aS)-3-{4-[4-(difluoromethoxy)phenyl]-1,3-thiazol-2-yl}-9-fluoro-1,3,4,11,12,12a-hexahydropyrido[1,2-b][2]benzazepin-6(2H)-one;
9-fluoro-3-[4-(thiophen-2-yl)-1,3-thiazol-2-yl]-1,3,4,11,12,12a-hexahydropyrido[1,2-b][2]benzazepin-6(2H)-one;
(3S,12aS)-9-fluoro-3-[4-(thiophen-2-yl)-1,3-thiazol-2-yl]-1,3,4,11,12,12a-hexahydropyrido[1,2-b][2]benzazepin-6(2H)-one;
3-[4-(5-chloropyrimidin-2-yl)-1,3-thiazol-2-yl]-9-fluoro-1,3,4,11,12,12a-hexahydropyrido[1,2-b][2]benzazepin-6(2H)-one;
(3S,12aS)-3-[4-(5-chloropyrimidin-2-yl)-1,3-thiazol-2-yl]-9-fluoro-1,3,4,11,12,12a-hexahydropyrido[1,2-b][2]benzazepin-6(2H)-one;
9-fluoro-3-[4-(4-fluorophenyl)-1,3-thiazol-2-yl]-1,3,4,11,12,12a-hexahydropyrido[1,2-b][2]benzazepin-6(2H)-one;
(3S,12aS)-9-fluoro-3-[4-(4-fluorophenyl)-1,3-thiazol-2-yl]-1,3,4,11,12,12a-hexahydropyrido[1,2-b][2]benzazepin-6(2H)-one;
9-fluoro-3-[4-(5-methylpyridin-2-yl)-1,3-thiazol-2-yl]-1,3,4,11,12,12a-hexahydropyrido[1,2-b][2]benzazepin-6(2H)-one;
(3S,12aS)-9-fluoro-3-[4-(5-methylpyridin-2-yl)-1,3-thiazol-2-yl]-1,3,4,11,12,12a-hexahydropyrido[1,2-b][2]benzazepin-6(2H)-one;
9-fluoro-3-[4-(5-methylpyrimidin-2-yl)-1,3-thiazol-2-yl]-1,3,4,11,12,12a-hexahydropyrido[1,2-b][2]benzazepin-6(2H)-one;
(3S,12aS)-9-fluoro-3-[4-(5-methylpyrimidin-2-yl)-1,3-thiazol-2-yl]-1,3,4,11,12,12a-hexahydropyrido[1,2-b][2]benzazepin-6(2H)-one;
3-[4-(5-bromopyrimidin-2-yl)-1,3-thiazol-2-yl]-9-fluoro-1,3,4,11,12,12a-hexahydropyrido[1,2-b][2]benzazepin-6(2H)-one;
(3S,12aS)-3-[4-(5-bromopyrimidin-2-yl)-1,3-thiazol-2-yl]-9-fluoro-1,3,4,11,12,12a-hexahydropyrido[1,2-b][2]benzazepin-6(2H)-one;
9-fluoro-3-[4-(5-fluoropyridin-2-yl)-1,3-thiazol-2-yl]-1,3,4,11,12,12a-hexahydropyrido[1,2-b][2]benzazepin-6(2H)-one;
(3S,12aS)-9-fluoro-3-[4-(5-fluoropyridin-2-yl)-1,3-thiazol-2-yl]-1,3,4,11,12,12a-hexahydropyrido[1,2-b][2]benzazepin-6(2H)-one;
9-chloro-3-[4-(pyridin-2-yl)-1,3-thiazol-2-yl]-1,3,4,11,12,12a-hexahydropyrido[1,2-b][2]benzazepin-6(2H)-one;
(3S,12aS)-9-chloro-3-[4-(pyridin-2-yl)-1,3-thiazol-2-yl]-1,3,4,11,12,12a-hexahydropyrido[1,2-b][2]benzazepin-6(2H)-one;
9-chloro-3-[4-(pyrimidin-2-yl)-1,3-thiazol-2-yl]-1,3,4,11,12,12a-hexahydropyrido[1,2-b][2]benzazepin-6(2H)-one;
(3S,12aS)-9-chloro-3-[4-(pyrimidin-2-yl)-1,3-thiazol-2-yl]-1,3,4,11,12,12a-hexahydropyrido[1,2-b][2]benzazepin-6(2H)-one;
3-[4-(5-bromopyridin-2-yl)-1,3-thiazol-2-yl]-9-fluoro-1,3,4,11,12,12a-hexahydropyrido[1,2-b][2]benzazepin-6(2H)-one;
(3S,12aS)-3-[4-(5-bromopyridin-2-yl)-1,3-thiazol-2-yl]-9-fluoro-1,3,4,11,12,12a-hexahydropyrido[1,2-b][2]benzazepin-6(2H)-one;
9-fluoro-3-[4-(4-methoxyphenyl)-1,3-thiazol-2-yl]-1,3,4,11,12,12a-hexahydropyrido[1,2-b][2]benzazepin-6(2H)-one;
(3S,12aS)-9-fluoro-3-[4-(4-methoxyphenyl)-1,3-thiazol-2-yl]-1,3,4,11,12,12a-hexahydropyrido[1,2-b][2]benzazepin-6(2H)-one;
9-fluoro-3-[4-(thiophen-3-yl)-1,3-thiazol-2-yl]-1,3,4,11,12,12a-hexahydropyrido[1,2-b][2]benzazepin-6(2H)-one;
(3S,12aS)-9-fluoro-3-[4-(thiophen-3-yl)-1,3-thiazol-2-yl]-1,3,4,11,12,12a-hexahydropyrido[1,2-b][2]benzazepin-6(2H)-one;
9-fluoro-3-[4-(5-fluoropyridin-2-yl)-1,3-thiazol-2-yl]-1,3,4,11,12,12a-hexahydropyrido[1,2-b][2]benzazepin-6(2H)-one;
(3R,12aS)-9-fluoro-3-[4-(5-fluoropyridin-2-yl)-1,3-thiazol-2-yl]-1,3,4,11,12,12a-hexahydropyrido[1,2-b][2]benzazepin-6(2H)-one;
3-[4-(5-bromopyridin-2-yl)-1,3-thiazol-2-yl]-9-fluoro-1,3,4,11,12,12a-hexahydropyrido[1,2-b][2]benzazepin-6(2H)-one;
(3R,12aS)-3-[4-(5-bromopyridin-2-yl)-1,3-thiazol-2-yl]-9-fluoro-1,3,4,11,12,12a-hexahydropyrido[1,2-b][2]benzazepin-6(2H)-one;
3-[4-(5-bromopyrimidin-2-yl)-1,3-thiazol-2-yl]-9-fluoro-1,3,4,11,12,12a-hexahydropyrido[1,2-b][2]benzazepin-6(2H)-one;
(3S,12aS)-3-[4-(5-bromopyrimidin-2-yl)-1,3-thiazol-2-yl]-9-fluoro-1,3,4,11,12,12a-hexahydropyrido[1,2-b][2]benzazepin-6(2H)-one;
9-fluoro-3-[4-(1H-pyrrol-2-yl)-1,3-thiazol-2-yl]-1,3,4,11,12,12a-hexahydropyrido[1,2-b][2]benzazepin-6(2H)-one;
(3S,12aS)-9-fluoro-3-[4-(1H-pyrrol-2-yl)-1,3-thiazol-2-yl]-1,3,4,11,12,12a-hexahydropyrido[1,2-b][2]benzazepin-6(2H)-one;
9-fluoro-3-[4-(pyrazin-2-yl)-1,3-thiazol-2-yl]-1,3,4,11,12,12a-hexahydropyrido[1,2-b][2]benzazepin-6(2H)-one;
(3S,12aS)-9-fluoro-3-[4-(pyrazin-2-yl)-1,3-thiazol-2-yl]-1,3,4,11,12,12a-hexahydropyrido[1,2-b][2]benzazepin-6(2H)-one;
9-fluoro-3-[4-(5-fluoropyrimidin-2-yl)-1,3-thiazol-2-yl]-1,3,4,11,12,12a-hexahydropyrido[1,2-b][2]benzazepin-6(2H)-one;
(3S,12aS)-9-fluoro-3-[4-(5-fluoropyrimidin-2-yl)-1,3-thiazol-2-yl]-1,3,4,11,12,12a-hexahydropyrido[1,2-b][2]benzazepin-6(2H)-one;
9-fluoro-3-[4-(5-fluoropyrimidin-2-yl)-1,3-thiazol-2-yl]-1,3,4,11,12,12a-hexahydropyrido[1,2-b][2]benzazepin-6(2H)-one; and
(3R,12aS)-9-fluoro-3-[4-(5-fluoropyrimidin-2-yl)-1,3-thiazol-2-yl]-1,3,4,11,12,12a-hexahydropyrido[1,2-b][2]benzazepin-6(2H)-one;
or a pharmaceutically acceptable salt thereof.

13. A pharmaceutical composition which comprises a pharmaceutically acceptable carrier and a compound of claim 1 or a pharmaceutically acceptable salt thereof.

14. (canceled)

15. (canceled)

16. A method for treating a neurological or psychiatric disorder associated with glutamate dysfunction in a patient in need thereof comprising administering to the patient a therapeutically effective amount of the compound of claim 1 or a pharmaceutically acceptable salt thereof.

17. A method for treating schizophrenia in a mammalian patient in need thereof which comprises administering to the patient a therapeutically effective amount of a compound of claim 1 or a pharmaceutically acceptable salt thereof.
